SEQUENCE LISTING (10 sequences)

SEQ ID NO: 1 - DNA, 4345 bp, Homo sapiens
  CDS (297)..(3908)
actgaaactaggggcaaggagacgaagagaacatgaaagttaaactttaagatgaagaacaaagctgaacatactgatgcattggatctttggagaggatctcagaactcattgtacttaatttacaggctaaaaccttagaagaggaatttattatatcctacacaagactccagggaagcacatggccttggactgaaggctggcatctggaagctgtcagccaccagcaccttctgcagcaggaaaaggccagggctctgctggagcaggcagcagagtggacgcacagtaacatgggcaacttgaagagcgtggcccaggagcctgggccaccctgcggcctggggctggggctgggccttgggctgtgcggcaagcagggcccagccaccccggcccctgagcccagccgggccccagcatccctactcccaccagcgccagaacacagccccccgagctccccgctaacccagcccccagaggggcccaagttccctcgtgtgaagaactgggaggtggggagcatcacctatgacaccctcagcgcccaggcgcagcaggatgggccctgcaccccaagacgctgcctgggctccctggtatttccacggaaactacagggccggccctcccccggccccccggcccctgagcagctgctgagtcaggcccgggacttcatcaaccagtactacagctccattaagaggagcggctcccaggcccacgaacagcggcttcaagaggtggaagccgaggtggcagccacaggcacctaccagcttagggagagcgagctggtgttcggggctaagcaggcctggcgcaacgctccccgctgcgtgggccggatccagtgggggaagctgcaggtgttcgatgcccgggactgcaggtctgcacaggaaatgttcacctacatctgcaaccacatcaagtatgccaccaaccggggcaaccttcgctcggccatcacagtgttcccgcagcgctgccctggccgaggagacttccgaatctggaacagccagctggtgcgctacgcgggctaccggcagcaggatggctctgtgcggggggacccagccaacgtggagatcaccgagctctgcattcagcacggctggaccccaggaaacggtcgcttcgacgtgctgcccctgctgctgcaggccccagatgagcccccagaactcttccttctgccccccgagctggtccttgaggtgcccctggagcaccccacgctggagtggtttgcagccctgggcctgcgctggtacgccctcccggcagtgtccaacatgctgctggaaattgggggcctggagttccccgcagcccccttcagtggctggtacatgagcactgagatcggcacgaggaacctgtgtgaccctcaccgctacaacatcctggaggatgtggctgtctgcatggacctggatacccggaccacctcgtccctgtggaaagacaaggcagcagtggaaatcaacgtggccgtgctgcacagttaccagctagccaaagtcaccatcgtggaccaccacgccgccacggcctctttcatgaagcacctggagaatgagcagaaggccagggggggctgccctgcagactgggcctggatcgtgccccccatctcgggcagcctcactcctgttttccatcaggagatggtcaactatttcctgtccccggccttccgctaccagccagacccctggaaggggagtgccgccaagggcaccggcatcaccaggaagaagacctttaaagaagtggccaacgccgtgaagatctccgcctcgctcatgggcacggtgatggcgaagcgagtgaaggcgacaatcctgtatggctccgagaccggccgggcccagagctacgcacagcagctggggagactcttccggaaggcttttgatccccgggtcctgtgtatggatgagtatgacgtggtgtccctcgaacacgagacgctggtgctggtggtaaccagcacatttgggaatggggatcccccggagaatggagagagctttgcagctgccctgatggagatgtccggcccctacaacagctcccctcggccggaacagcacaagagttataagatccgcttcaacagcatctcctgctcagacccactggtgtcctcttggcggcggaagaggaaggagtccagtaacacagacagtgcaggggccctgggcaccctcaggttctgtgtgttcgggctcggctcccgggcatacccccacttctgcgcctttgctcgtgccgtggacacacggctggaggaactgggcggggagcggctgctgcagctgggccagggcgacgagctgtgcggccaggaggaggccttccgaggctgggcccaggctgccttccaggccgcctgtgagaccttctgtgtgggagaggatgccaaggccgccgcccgagacatcttcagccccaaacggagctggaagcgccagaggtaccggctgagcgcccaggccgagggcctgcagttgctgccaggtctgatccacgtgcacaggcggaagatgttccaggctacaatccgctcagtggaaaacctgcaaagcagcaagtccacgagggccaccatcctggtgcgcctggacaccggaggccaggaggggctgcagtaccagccgggggaccacataggtgtctgcccgcccaaccggcccggccttgtggaggcgctgctgagccgcgtggaggacccgccggcgcccactgagcccgtggcagtagagcagctggagaagggcagccctggtggccctccccccggctgggtgcgggacccccggctgcccccgtgcacgctgcgccaggctctcaccttcttcctggacatcacctccccacccagccctcagctcttgcggctgctcagcaccttggcagaagagcccagggaacagcaggagctggaggccctcagccaggatccccgacgctacgaggagtggaagtggttccgctgccccacgctgctggaggtgctggagcagttcccgtcggtggcgctgcctgccccactgctcctcacccagctgcctctgctccagccccggtactactcagtcagctcggcacccagcacccacccaggagagatccacctcactgtagctgtgctggcatacaggactcaggatgggctgggccccctgcactatggagtctgctccacgtggctaagccagctcaagcccggagaccctgtgccctgcttcatccggggggctccctccttccggctgccacccgatcccagcttgccctgcatcctggtgggtccaggcactggcattgcccccttccggggattctggcaggagcggctgcatgacattgagagcaaagggctgcagcccactcccatgactttggtgttcggctgccgatgctcccaacttgaccatctctaccgcgacgaggtgcagaacgcccagcagcgcggggtgtttggccgagtcctcaccgccttctcccgggaacctgacaaccccaagacctacgtgcaggacatcctgaggacggagctggctgcggaggtgcaccgcgtgctgtgcctcgagcggggccacatgtttgtctgcggcgatgttaccatggcaaccaacgtcctgcagaccgtgcagcgcatcctggcgacggagggcgacatggagctggacgaggccggcgacgtcatcggcgtgctgcgggatcagcaacgctaccacgaagacattttcgggctcacgctgcgcacccaggaggtgacaagccgcatacgcacccagagcttttccttgcaggagcgtcagttgcggggcgcagtgccctgggcgttcgaccctcccggctcagacaccaacagcccctgagagccgcctggctttcccttccagttccgggagagcggctgcccgactcaggtccgcccgaccaggatcagccccgctcctcccctcttgaggtggtgccttctcacatctgtccagaggctgcaaggattcagcattattcctccaggaaggagcaaaacgcctcttttccctctctaggcctgttgcctcgggcctgggtccgccttaatctggaaggcccctcccagcagcggtaccccagggcctactgccacccgcttcctgtttcttagtcgaatgttagattcctcttgcctctctcaggagtatcttacctgtaaagtctaatctctaaatcaagtatttattattgaagatttaccataagggactgtgccagatgttaggagaactactaaagtgcctaccccagctcatgtggattacaaaaaaaa

SEQ ID NO: 2 - protein, 1203 aa, Homo sapiens
MGNLKSVAQEPGPPCGLGLGLGLGLCGKQGPATPAPEPSRAPASLLPPAPEHSPPSSPLTQPPEGPKFPRVKNWEVGSITYDTLSAQAQQDGPCTPRRCLGSLVFPRKLQGRPSPGPPAPEQLLSQARDFINQYYSSIKRSGSQAHEQRLQEVEAEVAATGTYQLRESELVFGAKQAWRNAPRCVGRIQWGKLQVFDARDCRSAQEMFTYICNHIKYATNRGNLRSAITVFPQRCPGRGDFRIWNSQLVRYAGYRQQDGSVRGDPANVEITELCIQHGWTPGNGRFDVLPLLLQAPDEPPELFLLPPELVLEVPLEHPTLEWFAALGLRWYALPAVSNMLLEIGGLEFPAAPFSGWYMSTEIGTRNLCDPHRYNILEDVAVCMDLDTRTTSSLWKDKAAVEINVAVLHSYQLAKVTIVDHHAATASFMKHLENEQKARGGCPADWAWIVPPISGSLTPVFHQEMVNYFLSPAFRYQPDPWKGSAAKGTGITRKKTFKEVANAVKISASLMGTVMAKRVKATILYGSETGRAQSYAQQLGRLFRKAFDPRVLCMDEYDVVSLEHETLVLVVTSTFGNGDPPENGESFAAALMEMSGPYNSSPRPEQHKSYKIRFNSISCSDPLVSSWRRKRKESSNTDSAGALGTLRFCVFGLGSRAYPHFCAFARAVDTRLEELGGERLLQLGQGDELCGQEEAFRGWAQAAFQAACETFCVGEDAKAAARDIFSPKRSWKRQRYRLSAQAEGLQLLPGLIHVHRRKMFQATIRSVENLQSSKSTRATILVRLDTGGQEGLQYQPGDHIGVCPPNRPGLVEALLSRVEDPPAPTEPVAVEQLEKGSPGGPPPGWVRDPRLPPCTLRQALTFFLDITSPPSPQLLRLLSTLAEEPREQQELEALSQDPRRYEEWKWFRCPTLLEVLEQFPSVALPAPLLLTQLPLLQPRYYSVSSAPSTHPGEIHLTVAVLAYRTQDGLGPLHYGVCSTWLSQLKPGDPVPCFIRGAPSFRLPPDPSLPCILVGPGTGIAPFRGFWQERLHDIESKGLQPTPMTLVFGCRCSQLDHLYRDEVQNAQQRGVFGRVLTAFSREPDNPKTYVQDILRTELAAEVHRVLCLERGHMFVCGDVTMATNVLQTVQRILATEGDMELDEAGDVIGVLRDQQRYHEDIFGLTLRTQEVTSRIRTQSFSLQERQLRGAVPWAFDPPGSDTNSP

SEQ ID NO: 3 - DNA, 4345 bp, Homo sapiens
  CDS (297)..(3908)
actgaaactaggggcaaggagacgaagagaacatgaaagttaaactttaagatgaagaacaaagctgaacatactgatgcattggatctttggagaggatctcagaactcattgtacttaatttacaggctaaaaccttagaagaggaatttattatatcctacacaagactccagggaagcacatggccttggactgaaggctggcatctggaagctgtcagccaccagcaccttctgcagcaggaaaaggccagggctctgctggagcaggcagcagagtggacgcacagtaacatgggcaacttgaagagcgtggcccaggagcctgggccaccctgcggcctggggctggggctgggccttgggctgtgcggcaagcagggcccagccaccccggcccctgagcccagccgggccccagcatccctactcccaccagcgccagaacacagccccccgagctccccgctaacccagcccccagaggggcccaagttccctcgtgtgaagaactgggaggtggggagcatcacctatgacaccctcagcgcccaggcgcagcaggatgggccctgcaccccaagacgctgcctgggctccctggtatttccacggaaactacagggccggccctcccccggccccccggcccctgagcagctgctgagtcaggcccgggacttcatcaaccagtactacagctccattaagaggagcggctcccaggcccacgaacagcggcttcaagaggtggaagccgaggtggcagccacaggcacctaccagcttagggagagcgagctggtgttcggggctaagcaggcctggcgcaacgctccccgctgcgtgggccggatccagtgggggaagctgcaggtgttcgatgcccgggactgcaggtctgcacaggaaatgttcacctacatctgcaaccacatcaagtatgccaccaaccggggcaaccttcgctcggccatcacagtgttcccgcagcgctgccctggccgaggagacttccgaatctggaacagccagctggtgcgctacgcgggctaccggcagcaggatggctctgtgcggggggacccagccaacgtggagatcaccgagctctgcattcagcacggctggaccccaggaaacggtcgcttcgacgtgctgcccctgctgctgcaggccccagatgatcccccagaactcttccttctgccccccgagctggtccttgaggtgcccctggagcaccccacgctggagtggtttgcagccctgggcctgcgctggtacgccctcccggcagtgtccaacatgctgctggaaattgggggcctggagttccccgcagcccccttcagtggctggtacatgagcactgagatcggcacgaggaacctgtgtgaccctcaccgctacaacatcctggaggatgtggctgtctgcatggacctggatacccggaccacctcgtccctgtggaaagacaaggcagcagtggaaatcaacgtggccgtgctgcacagttaccagctagccaaagtcaccatcgtggaccaccacgccgccacggcctctttcatgaagcacctggagaatgagcagaaggccagggggggctgccctgcagactgggcctggatcgtgccccccatctcgggcagcctcactcctgttttccatcaggagatggtcaactatttcctgtccccggccttccgctaccagccagacccctggaaggggagtgccgccaagggcaccggcatcaccaggaagaagacctttaaagaagtggccaacgccgtgaagatctccgcctcgctcatgggcacggtgatggcgaagcgagtgaaggcgacaatcctgtatggctccgagaccggccgggcccagagctacgcacagcagctggggagactcttccggaaggcttttgatccccgggtcctgtgtatggatgagtatgacgtggtgtccctcgaacacgagacgctggtgctggtggtaaccagcacatttgggaatggggatcccccggagaatggagagagctttgcagctgccctgatggagatgtccggcccctacaacagctcccctcggccggaacagcacaagagttataagatccgcttcaacagcatctcctgctcagacccactggtgtcctcttggcggcggaagaggaaggagtccagtaacacagacagtgcaggggccctgggcaccctcaggttctgtgtgttcgggctcggctcccgggcatacccccacttctgcgcctttgctcgtgccgtggacacacggctggaggaactgggcggggagcggctgctgcagctgggccagggcgacgagctgtgcggccaggaggaggccttccgaggctgggcccaggctgccttccaggccgcctgtgagaccttctgtgtgggagaggatgccaaggccgccgcccgagacatcttcagccccaaacggagctggaagcgccagaggtaccggctgagcgcccaggccgagggcctgcagttgctgccaggtctgatccacgtgcacaggcggaagatgttccaggctacaatccgctcagtggaaaacctgcaaagcagcaagtccacgagggccaccatcctggtgcgcctggacaccggaggccaggaggggctgcagtaccagccgggggaccacataggtgtctgcccgcccaaccggcccggccttgtggaggcgctgctgagccgcgtggaggacccgccggcgcccactgagcccgtggcagtagagcagctggagaagggcagccctggtggccctccccccggctgggtgcgggacccccggctgcccccgtgcacgctgcgccaggctctcaccttcttcctggacatcacctccccacccagccctcagctcttgcggctgctcagcaccttggcagaagagcccagggaacagcaggagctggaggccctcagccaggatccccgacgctacgaggagtggaagtggttccgctgccccacgctgctggaggtgctggagcagttcccgtcggtggcgctgcctgccccactgctcctcacccagctgcctctgctccagccccggtactactcagtcagctcggcacccagcacccacccaggagagatccacctcactgtagctgtgctggcatacaggactcaggatgggctgggccccctgcactatggagtctgctccacgtggctaagccagctcaagcccggagaccctgtgccctgcttcatccggggggctccctccttccggctgccacccgatcccagcttgccctgcatcctggtgggtccaggcactggcattgcccccttccggggattctggcaggagcggctgcatgacattgagagcaaagggctgcagcccactcccatgactttggtgttcggctgccgatgctcccaacttgaccatctctaccgcgacgaggtgcagaacgcccagcagcgcggggtgtttggccgagtcctcaccgccttctcccgggaacctgacaaccccaagacctacgtgcaggacatcctgaggacggagctggctgcggaggtgcaccgcgtgctgtgcctcgagcggggccacatgtttgtctgcggcgatgttaccatggcaaccaacgtcctgcagaccgtgcagcgcatcctggcgacggagggcgacatggagctggacgaggccggcgacgtcatcggcgtgctgcgggatcagcaacgctaccacgaagacattttcgggctcacgctgcgcacccaggaggtgacaagccgcatacgcacccagagcttttccttgcaggagcgtcagttgcggggcgcagtgccctgggcgttcgaccctcccggctcagacaccaacagcccctgagagccgcctggctttcccttccagttccgggagagcggctgcccgactcaggtccgcccgaccaggatcagccccgctcctcccctcttgaggtggtgccttctcacatctgtccagaggctgcaaggattcagcattattcctccaggaaggagcaaaacgcctcttttccctctctaggcctgttgcctcgggcctgggtccgccttaatctggaaggcccctcccagcagcggtaccccagggcctactgccacccgcttcctgtttcttagtcgaatgttagattcctcttgcctctctcaggagtatcttacctgtaaagtctaatctctaaatcaagtatttattattgaagatttaccataagggactgtgccagatgttaggagaactactaaagtgcctaccccagctcatgtggattacaaaaaaaa

SEQ ID NO: 4 - protein, 1203 aa, Homo sapiens
MGNLKSVAQEPGPPCGLGLGLGLGLCGKQGPATPAPEPSRAPASLLPPAPEHSPPSSPLTQPPEGPKFPRVKNWEVGSITYDTLSAQAQQDGPCTPRRCLGSLVFPRKLQGRPSPGPPAPEQLLSQARDFINQYYSSIKRSGSQAHEQRLQEVEAEVAATGTYQLRESELVFGAKQAWRNAPRCVGRIQWGKLQVFDARDCRSAQEMFTYICNHIKYATNRGNLRSAITVFPQRCPGRGDFRIWNSQLVRYAGYRQQDGSVRGDPANVEITELCIQHGWTPGNGRFDVLPLLLQAPDDPPELFLLPPELVLEVPLEHPTLEWFAALGLRWYALPAVSNMLLEIGGLEFPAAPFSGWYMSTEIGTRNLCDPHRYNILEDVAVCMDLDTRTTSSLWKDKAAVEINVAVLHSYQLAKVTIVDHHAATASFMKHLENEQKARGGCPADWAWIVPPISGSLTPVFHQEMVNYFLSPAFRYQPDPWKGSAAKGTGITRKKTFKEVANAVKISASLMGTVMAKRVKATILYGSETGRAQSYAQQLGRLFRKAFDPRVLCMDEYDVVSLEHETLVLVVTSTFGNGDPPENGESFAAALMEMSGPYNSSPRPEQHKSYKIRFNSISCSDPLVSSWRRKRKESSNTDSAGALGTLRFCVFGLGSRAYPHFCAFARAVDTRLEELGGERLLQLGQGDELCGQEEAFRGWAQAAFQAACETFCVGEDAKAAARDIFSPKRSWKRQRYRLSAQAEGLQLLPGLIHVHRRKMFQATIRSVENLQSSKSTRATILVRLDTGGQEGLQYQPGDHIGVCPPNRPGLVEALLSRVEDPPAPTEPVAVEQLEKGSPGGPPPGWVRDPRLPPCTLRQALTFFLDITSPPSPQLLRLLSTLAEEPREQQELEALSQDPRRYEEWKWFRCPTLLEVLEQFPSVALPAPLLLTQLPLLQPRYYSVSSAPSTHPGEIHLTVAVLAYRTQDGLGPLHYGVCSTWLSQLKPGDPVPCFIRGAPSFRLPPDPSLPCILVGPGTGIAPFRGFWQERLHDIESKGLQPTPMTLVFGCRCSQLDHLYRDEVQNAQQRGVFGRVLTAFSREPDNPKTYVQDILRTELAAEVHRVLCLERGHMFVCGDVTMATNVLQTVQRILATEGDMELDEAGDVIGVLRDQQRYHEDIFGLTLRTQEVTSRIRTQSFSLQERQLRGAVPWAFDPPGSDTNSP

SEQ ID NO: 5 - DNA, 22 bp, Artificial Sequence
  Synthetic primer
aaggcaggagacagtggatgga

SEQ ID NO: 6 - DNA, 24 bp, Artificial Sequence
  Synthetic primer
cccagtcaatccctttggtgctca

SEQ ID NO: 7 - DNA, 22 bp, Artificial Sequence
  Synthetic primer
tggagagtgctggtgtacccca

SEQ ID NO: 8 - DNA, 20 bp, Artificial Sequence
  Synthetic primer
gcctccacccccaccctgtc

SEQ ID NO: 9 - DNA, 21 bp, Artificial Sequence
  Synthetic primer
aggccctatggtagtgccttt

SEQ ID NO: 10 - DNA, 20 bp, Artificial Sequence
  Synthetic primer
tctcttagtgctgtggtcac

(19) United States
(12) Patent Application Publication
(10) Pub. No.: US 2013/0131161 A1
(43) Pub. Date: May 23, 2013

(54) METHODS AND COMPOSITIONS FOR CARDIOVASCULAR DISEASES AND CONDITIONS

(71) Applicants: Michael R. Bristow, Englewood, CO (US); Jonathan David Port, Denver, CO (US)

(72) Inventors: Michael R. Bristow, Englewood, CO (US); Jonathan David Port, Denver, CO (US)

(21) Appl. No.: 13/518,471

(22) PCT Filed: Dec. 23, 2010
(86) PCT No.: PCT/US10/62023

§ 371 Date: Feb. 11, 2013

Related U.S. Application Data
(60) Provisional application No. 61/289,932, filed on Dec. 23, 2009.

(51) Int. Cl. A61K 31/34

(52) U.S. Cl. CPC A61K 31/34
USPC 514/470

(57) ABSTRACT

Embodiments of the invention are directed to identifying or treating a patient that would benefit from phosphodiesterase inhibitor therapy.

This application claims the benefit of priority to U.S. Provisional Patent Application Ser. No. 61/289,932, filed on Dec. 23, 2009, which is hereby incorporated by reference in its entirety.

### BACKGROUND OF THE INVENTION

I. Field of the Invention

Embodiments of this invention are directed generally to biology, molecular genetics, and medicine. Certain embodiments are directed to methods and compositions involving identifying and/or treating a patient that would benefit from therapy with a compound called with S-(6-Nitro-oxi-hexahydro-furo[3,2-b]furan-3-1-il)thioacetate, which is also known as LA-419.

II. Background

Heart failure, or end-stage CV disease, affects approximately five million Americans. Epidemiologic studies indicate a higher prevalence of risk factors for heart failure among African Americans in the United States (Burt et al., 1995). Recent analyses of heart failure clinical trials show that the mortality rate and hospitalization rate for African Americans are significantly higher than for non-African Americans.

The thin layer of cells that lines the interior surface of blood vessels is called the endothelium. This layer modulates vascular tone through the release of nitric oxide (NO), a potent vasodilator that regulates regional blood flow (Ignarro et al., 1987; Rees et al., 1989). A reduction in NO bioavailability contributes to elevated vascular resistance and loss of sensitivity to stimuli of vasodilation, hallmark features of hypertension (Paniagua et al., 2001; Panza et al., 1990; Taddei et al., 1993). In addition, NO has well characterized vascular benefits such as inhibition of smooth muscle cell proliferation and migration, blocking adhesion of leukocytes to the endothelium, and preventing platelet aggregation (Harrison, 1997). Agents that directly stimulate NO release have been investigated for the prevention and treatment of cardiovascular (CV) diseases and conditions.

In the United States, African Americans exhibit a higher prevalence of CV risk factors, especially hypertension (Burt et al., 1995). One theory is that this may be attributed to differences in vascular physiology, including reduced NO bioavailability. Support for this was provided by a clinical evaluation of brachial artery activity that demonstrated reduced responsiveness of conductance vessels to both endogenous and exogenous NO in healthy African Americans, as compared with age-matched Whites (Campia et al., 2002). As an explanation of this, it was reported that there is low bioavailability of NO from endothelium of African Americans, despite much higher levels of endothelial-dependent NO synthase (eNOS) (Kalinowski et al., 2004). The cellular basis for this paradox was the finding that excessive O₂⁻ generation by NAD(P)H oxidase and uncoupled eNOS resulted in the loss of functional NO due to its reactivity with O₂⁻, resulting in peroxynitrite (ONOO⁻) formation, a potent oxidant. It has recently been demonstrated that nebivolol, unlike another β₁-selective inhibitor (atenolol), is able to effectively reduce nitroxidative stress and restore NO bioavailability in African Americans (Mason et al., 2005).

BiDil is a heart failure drug that has been recently approved for treatment in African Americans. It consists of a fixed dose combination of isosorbide dinitrate and hydralazine. Isosorbide dinitrate is a direct NO donor, and extended use causes tolerance. Hydralazine lowers blood pressure by relaxing vascular smooth muscle cells. This may be caused by reductions in inositol triphosphate levels, a second messenger that stimulates calcium release from the sarcoplasmic reticulum of smooth muscle.

The vasodilating effect of hydralazine leads to 1) decreased arterial blood pressure (diastolic more than systolic); 2) decreased peripheral vascular resistance; and, 3) increased heart rate, stroke volume, and cardiac output. Hydralazine may increase renin activity in plasma, presumably caused by increased secretion of renin in response to reflex sympathetic release. This increase in renin activity results in the production of angiotensin II, a stimulus for aldosterone with consequent sodium reabsorption. Hydalazine also has putative antioxidant activity that may contribute to reduced loss of NO.

Nitrates such as isosorbide dinitrate are direct NO donors that are linked to activation of guanylate cyclase, a mediator of smooth muscle cell vasodilation. They have been widely used for decades in the treatment of angina pectoris and hypertension, but work independently of the endothelium. In the formulation of sodium nitroprusside, NO is coordinated as a nitrosyl group liganded to iron in a square bipyramidal complex that is released spontaneously at physiological pH. A major disadvantage associated with extended use of nitrates is tolerance, leading to enhanced vascular O₂⁻ production (Münzel et al., 1995). Thus, there is a need for therapies that can enhance NO levels in the vessel wall without causing tolerance and enhancing oxidative stress.

### SUMMARY OF THE INVENTION

The ability to identify which drug to treat a patient with is critical to the proper treatment of that patient. Embodiments of the invention can be used for diagnostic and treatment of patients having a cardiovascular disease or condition. In certain embodiments, the treatment involves an NO donor drug. In particular embodiments, treatment involves S-(6-Nitro-oxi-hexahydro-furo[3,2-b]furan-3-1-il)thioacetate (LA-419) or a salt, metabolite, or derivative thereof. It is contemplated that any embodiment involving LA-419, may also or alternatively involve a salt or prodrug thereof in other embodiments recited herein.

In some embodiments there are methods for treating a cardiovascular disease or condition in a patient comprising administering to the patient an effective amount of pharmaceutical composition comprising the compound S-(6-Nitro-oxi-hexahydro-furo[3,2-b]furan-3-1-il)thioacetate. Some embodiments concern a patient that has been tested and determined to be homozygous wildtype (G/G) at position 894 in the endothelial nitric oxide synthase (NOS3) gene. A patient may be administered LA-419 after it is known that the patient is homozygous wildtype (G/G) at position 894 of the NOS3 gene.

Embodiments also include methods for treating a patient with S-(6-Nitro-oxi-hexahydro-furo[3,2-b]furan-3-1-il)thioacetate comprising administering to the patient an effective amount of S-(6-Nitro-oxi-hexahydro-furo[3,2-b]furan-3-1-il)thioacetate after the patient is tested and determined to be homozygous wildtype (G/G) at position-894 in the endothelial nitric oxide synthase (NOS3) gene.

Other embodiments involve methods for increasing nitric oxide (NO) levels in a patient comprising administering to the patient an effective amount of S-(6-Nitro-oxi-hexahydro-furo[3,2-b]furan-3-1-il)thioacetate after the patient is tested and determined to be homozygous wildtype (G/G) at position 894 in the endothelial nitric oxide synthase (NOS3) gene.

In further embodiments there are methods for evaluating whether LA-419 is an appropriate therapy for a patient who has been diagnosed with or has symptoms of a cardiovascular disease or condition. Any embodiment may involve determining whether the patient is homozygous wildtype at position 894 (G/G) in the NOS3 gene. This determination may involve directly determining the sequence by genotyping the patient based on a test performed on a biological sample containing nucleic acids from the patient that determines the sequence at position 894 in both alleles. This genotyping involves analyzing the nucleic acids in a sample using chemical techniques well known to those of skill in the art. In some embodiments, the determination is indirect and involves, for instance, reading the results of a report or database that reveals the sequence at that position is at both alleles (either by reporting the patient is homozygous for a residue—for example, G/G or T/T—or that the patient is heterozygous at that residue).

In some embodiments there are methods for predicting efficacy of LA-419 in a patient with a cardiovascular disease or condition or with symptoms thereof. In further embodiments there are methods for diagnosing a patient as a suitable candidate for treatment with S-(6-Nitro-oxi-hexahydro-furo[3,2-b]furan-3-1-il)thioacetate comprising assaying a sample from the patient to determine whether the patient is homozygous wildtype (G/G) at position 894 in the endothelial nitric oxide synthase (NOS3) gene and reporting if the patient homozygous wildtype (G/G) at position 894 in the NOS3 gene.

In some embodiments, the cardiovascular disease or condition is a chronic ischemic cardiovascular disorder. In additional embodiments, the cardiovascular disease or condition is peripheral arterial disease, heart failure, coronary heart disease, coronary spasm, myocardial infarction, atherosclerosis, or pulmonary arterial hypertension.

Some embodiments involve the administration of an effective amount of LA-419 to the patient such that the patient may achieve a therapeutic benefit from LA-419. In some embodiments, the amount of LA-419 administered to a patient is sufficient to provide a serum level of a LA-419 metabolite of at most, at least, or about 10, 50, 100, 150, 200, 250, 300, 350, 400, 450, 500, 550, 600, 650, 700, 750, 800, 850, 900, 950, 1000 ng/ml and/or mg/ml, or any range derivable therein.

In some methods, the patient is administered at least one dose of the composition. The dose in the composition may include about 1-40 mg of LA-419. In some embodiments, the patient is administered at least one dose of the composition, wherein the composition comprises about 5-20 mg of the compound. In certain embodiments, the patient is administered multiple doses of the composition. It is contemplated that in further embodiments, the patient is administered multiple doses of the composition in a 24 hour period. Embodiments may involve a patient who is administered 1, 2, 3, or 4 doses of the composition, wherein the composition comprises about 1-40 mg of the compound. These doses may be administered in a 24 hour period or in a day. The NO donor, such as LA-419, can be administered at a dose of about, at least about, or at most about 0.01, 0.02, 0.03, 0.04, 0.05, 0.06, 0.07, 0.08, 009, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 50, 60, 70, 80, 90, 100, 110, 120, 130, 140, or 150 mg or mg/kg including all values and ranges there between. In certain aspects LA 419 or an NO donor is administered 1, 2, 3, 4, 5, 6 or more times a day, a week, or a month, including all values and ranges there between. Alternatively, the drug may be administered on an as needed basis based on the patient's symptoms.

In certain embodiments, an LA-419 metabolite is administered to the patient. In some embodiments, the amount of LA-419 metabolite is the same as the dosage and regimen for LA-419. In some embodiments, the dosage is 0.5×, 1.5×, 2×, 2.5×, 3×, 4× or more greater than the dosage of LA-419.

Embodiments may include obtaining, providing or furnishing a biological sample from the patient for testing to determine the patient's genotype at position 894 in the NOS3 gene. A biological sample can be a blood sample, a buccal smear, a tissue sample, or a primary culture of somatic cells from the patient. In certain aspects analyzing the sample comprises performing nucleic acid sequencing, restriction digestion, allele-specific nucleic acid amplification, single-stranded conformational polymorphism analysis, or allele specific hybridization analysis. The methods described herein can further comprising preparing a report containing information regarding the genotype of one or more NOS3 genes of the patient. In a further aspect the patient is determined to be homozygous wildtype at position 894 in the NOS3 gene and is subsequently treated with LA-419. In other embodiments, the patient is determined to be heterozygous or homozygous T/T at position 894 of the NOS3 gene. In embodiments, a patient is not treated with LA-419 and may be treated with an alternative NO donor.

In some methods, a step of ordering a test that determines the patient's genotype at position 894 in the NOS3 gene is included. In further embodiments, the test results may be obtained by or provided to a clinician, such as one who is considered LA-419 treatment for a patient.

Embodiments may concern patients who have been diagnosed with a disease or condition associated with NO production. In some embodiments, a patient has been diagnosed with a cardiovascular disease or condition and/or the patient is exhibiting one or more symptoms of a cardiovascular disease or condition. In some embodiments, the disease is an ischemic cardiovascular disorder, glaucoma, or an intestinal disorder.

In some embodiments the nucleic acid sequence can be determined using nucleic acid amplification, nucleic acid hybridization, restriction fragment length polymorphism (RFLP) analysis, single stranded conformational polymorphism (SSCP) analysis, nucleic acid sequencing, denaturing high performance liquid chromatography, comparative genome hybridization, and/or Southern blotting. In certain aspects nucleic acid amplification comprises polymerase chain reaction amplification or ligase chain reaction amplification. In a further aspect nucleic acid hybridization detection method comprises an allele specific oligonucleotide probe or a microarray of nucleic acid probes.

Certain embodiments are directed to an isolated nucleic acid sequence comprising 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 600, 700, 800, 900, 1000 or more consecutive nucleotides of SEQ ID NO:1 or SEQ ID NO:3, including all values and ranges there between.

A further embodiment is directed to an amplification primer pair comprising two oligonucleotides that amplify a nucleic acid segment comprising nucleotide 894 of the human NOS3 gene, such as in SEQ ID NO:1 or SEQ ID NO:3, or a nucleic acid that is 85, 90, 95, 98, or 100% identical to SEQ ID NO:1 or 3 In some embodiments, a primer may comprise all or part of any of SEQ ID NOs:1, 3, or any of 5-10. For example, a first primer can comprise the nucleic acid sequence of SEQ ID NO:5. A second primer can comprise the nucleic acid sequence of SEQ ID NO:6.

Certain embodiments are directed to a nucleic acid probe that specifically hybridizes to a NOS3 nucleic acid comprising nucleotide 894 of SEQ ID NO:1 or SEQ ID NO:3 or a nucleic acid that is, is at least, or is at most, 85, 90, 95, 98, or 100% (or any range derivable therein) identical to one or both sequences. In certain aspects the nucleic acid probe is labeled. In a further aspect the nucleic acid probe is detectable upon binding or hybridization to a NOS3 nucleic acid comprising either the wildtype homozygous genotype or a genotype that is not homozygous wildtype at position 894 of the NOS3 gene.

Another aspect is a kit for genotyping a NOS3 gene comprising oligonucleotides of at least 10 contiguous nucleotides of SEQ ID NO:1 or SEQ ID NO:3 that amplify a nucleic acid segment comprising the 894 polymorphism or wildtype sequence at position 894, or a nucleic acid probe that specifically hybridizes to a NOS3 gene comprising the non-wild-type sequence at position 894.

To achieve these methods, a doctor, medical practitioner, or their staff may obtain a biological sample for evaluation. The sample may be analyzed by the practitioner or their staff, or it may be sent to an outside or independent laboratory. The medical practitioner may be cognizant of whether the test is providing information regarding the patient's NOS3 genes, or the medical practitioner may be aware only that the test indicates directly or indirectly that the genotype of the patient reflects the homozygous wildtype sequence in the NOS3 genes (homozygous for wildtype sequence).

Similarly, the medical practitioner may be cognizant of whether the test is providing information regarding the patient's NOS3 genes or the medical practitioner may be aware only that the test indicates directly or indirectly that the genotype of the patient reflects the homozygous wildtype sequence (G/G), heterozygous alleles (G/T or T/G), or the homozygous nonwildtype alleles (T/T).

In any of these circumstances, the medical practitioner “knows” or identifies indirectly the relevant information that will allow him or her to determine whether a NO donor such as LA-419 is an appropriate medical treatment. It is contemplated that, for example, a laboratory conducts the test to determine that patient's genotype such its personnel also know the appropriate information. They may report back to the practitioner with the specific result of the test performed or the laboratory may simply report that LA-419 is an appropriate drug based on the laboratory results. Moreover, through these different channels, the patient's genotype at position 894 of one or both NOS3 genes can be known.

Certain embodiments are directed to a tangible, computer-readable medium comprising a genotype of a subject, wherein the genotype exhibits the sequence at position 894 in one or both alleles of the NOS3 gene. In certain aspects the medium comprising the genotype of the subject exhibits the presence of the wildtype sequence at position 894 of one or more NOS3 gene.

In additional embodiments, there are kits for determining the nucleotide sequence either directly or indirectly. Embodiments for directly determining the nucleotide sequence may involve a kit in a suitable container comprising a probe or at least one set of primers for identifying the NOS3 polymorphism at position 894. In some embodiments, the probe comprises at least one nucleic acid of between 15 and 100 nucleotides of SEQ ID NO:1 and/or SEQ ID NO:3, wherein the probe includes the nucleotide at position 894 of the NOS3 gene. In other embodiments, kits involve primers comprising at least one set of primers for amplifying a region of sequence that includes position 894 of the NOS3 gene. Indirect methods may involve researching a database to determine the sequence or evaluating a protein sequence of the protein encoded by the patient's NOS3 alleles.

As used herein, the term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. Though the precise physiological mechanisms of heart failure are not entirely understood, heart failure is generally believed to involve disorders in several cardiac autonomic properties, including sympathetic, parasympathetic, and baroreceptor responses. The phrase “manifestations of heart failure” is used broadly to encompass all of the sequelae associated with heart failure, such as shortness of breath, pitting edema, an enlarged tender liver, engorged neck veins, pulmonary rates and the like including laboratory findings associated with heart failure.

The term “treatment” or equivalents encompasses the improvement and/or reversal of the symptoms of heart failure (i.e., the ability of the heart to pump blood). “Improvement in the physiologic function” of the heart may be assessed using any of the measurements described herein (e.g., measurement of ejection fraction, fractional shortening, left ventricular internal dimension, heart rate, etc.), as well as any effect upon subject's survival.

The term “dilated cardiomyopathy” refers to a type of heart failure characterized by the presence of a symmetrically dilated left ventricle with poor systolic contractile function and, in addition, frequently involves the right ventricle.

As used herein, the term “cardiac hypertrophy” refers to the process in which adult cardiac myocytes respond to stress through hypertrophic growth. Such growth is characterized by cell size increases without cell division, assembling of additional sarcomeres within the cell to attempt to increase force generation, and an activation of a fetal cardiac gene program that inherently reduces myocardial function. Cardiac hypertrophy is often associated with increased risk of morbidity and mortality, and thus studies aimed at understanding the molecular mechanisms of cardiac hypertrophy could have a significant impact on human health.

As used herein, the term “genotype” refers to the actual genetic make-up of an organism, while “phenotype” refers to physical traits displayed by an individual (responsiveness to LA-419 for the treatment of a chonic ischemic cardiovascular disease or condition).

Other embodiments are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Schematic diagram of a NO nanosensor placed in close proximity to the surface of a single endothelial cell. The nanosensor measures the levels of NO, O₂⁻, and ONOO⁻ from the intact endothelium in real time. The sensors are made by depositing a sensing material on the tip of carbon fiber with a diameter of approximately 0.5 p.m. The fibers are sealed with nonconductive epoxy and electrically connected to wires (gold, copper) with conductive silver epoxy.

FIG. 2. Comparative effects of LA-419 and ISDN on NO release from HUVECs isolated from non-Hispanic white donors. Cells were incubated with LA-419 or ISDN, each at 500 nM, for 12 hours prior to stimulation with CaI (1.0 μM). Values are reported as mean±S.D. (N=5). *p<0.05 vs. control (Dunnett multiple comparisons test; overall ANOVA: p=0.0015; F=10.326); ^{†}p=0.0129 vs. LA-419 treatment (Student t-test).

FIG. 3. Comparative effects of LA-419 and ISDN on ONOO⁻ release from HUVECs isolated from non-Hispanic white donors. Cells were incubated with LA-419 or ISDN, each at 500 nM, for 12 hours prior to stimulation with CaI (1.0 μM). Values are reported as mean±S.D. (N=5). *p<0.01 vs. control (Dunnett multiple comparisons test; overall ANOVA: p<0.0001; F=34.909). Student t-test analysis of isosorbide dinitrate vs. LA-419 treatment: p=0.0995.

FIG. 4. Comparative effects of LA-419 and ISDN on the ratio of NO/ONOO⁻ release from HUVECs isolated from non-Hispanic white donors. Cells were incubated with LA-419 or ISDN, each at 500 nM, for 12 hours prior to stimulation with CaI (1.0 μM). Values are reported as mean±S.D. (N=5). *p<0.01 vs. control (Dunnett multiple comparisons test; overall ANOVA: p<0.0001; F=20.154); ^{†}p=0.0024 vs. LA-419 treatment (Student t-test).

FIG. 5. Comparative effects of LA-419 and ISDN on NO release from HUVECs isolated from African American donors. Cells were incubated with LA-419 or ISDN, each at 500 nM, for 12 hours prior to stimulation with CaI (1.0 μM). Values are reported as mean±S.D. (N=5). *p<0.01 vs. control (Dunnett multiple comparisons test; overall ANOVA: p=0.0036; F=8.361); ^{†}p=0.0252 vs. LA-419 treatment (Student t-test).

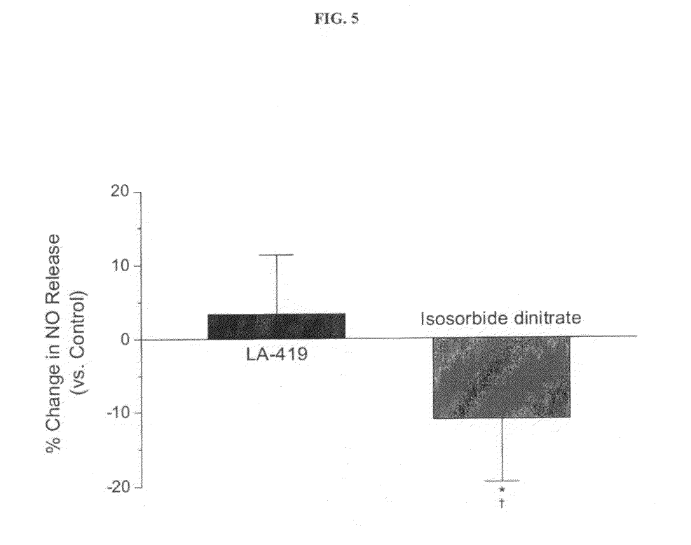

FIG. 6. Comparative effects of LA-419 and ISDN on ONOO⁻ release from HUVECs isolated from African American donors. Cells were incubated with LA-419 or ISDN, each at 500 nM, for 12 hours prior to stimulation with CaI (1.0 μM). Values are reported as mean±S.D. (N=5). *p<0.01 vs. control (Dunnett multiple comparisons test; overall ANOVA: p<0.0001; F=21.422). Student t-test analysis of isosorbide dinitrate vs. LA-419 treatment: p=0.4034.

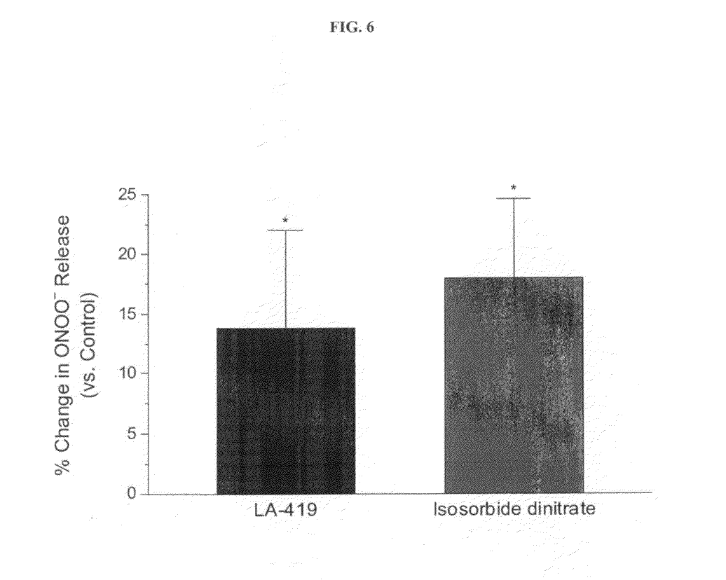

FIG. 7. Comparative effects of LA-419 and ISDN on the ratio of NO/ONOO⁻ release from HUVECs isolated from African American donors. Cells were incubated with LA-419 or ISDN, each at 500 nM, for 12 hours prior to stimulation with CaI (1.0 μM). Values are reported as mean±S.D. (N=5). *p<0.01 vs. control (Dunnett multiple comparisons test; overall ANOVA: p=0.0001; F=17.921); ^{†}p=0.0476 vs. LA-419 treatment (Student t-test).

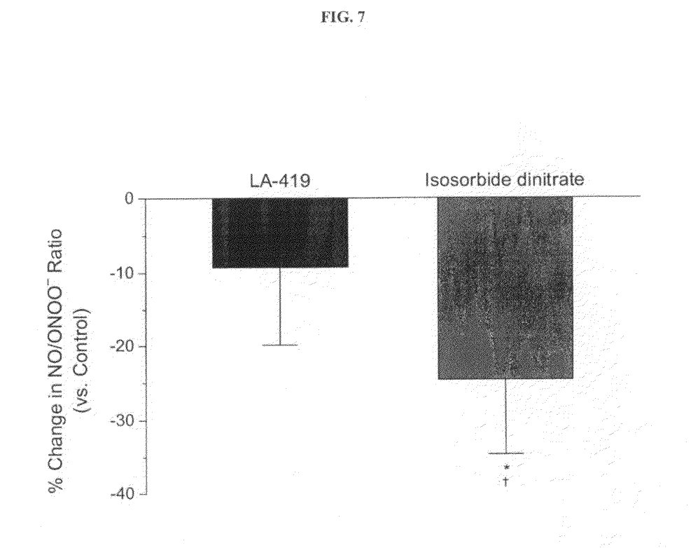

### DETAILED DESCRIPTION OF THE INVENTION

Treatment with drugs that act as NO donors is widespread. However, their efficacy is limited because of the tolerance they induce in patients. Ideally, a drug that did not induce the tolerance currently observed would be used to treat patients in need of the effects of nitric oxide. As shown below, S-(6-Nitro-oxi-hexahydro-furo[3,2-b]furan-3-1-il)thioacetate (LA-419) is demonstrated to have these ideal properties. Compositions and methods described herein involve treatment of patients with LA-419, particularly, in some embodiments, patients whose genotype is homozygous wildtype (G/G) at position 894 in the endothelial nitric oxide synthase gene, NOS3.

Embodiments relate generally to determining a NOS3 polymorphism in an individual and determining responsiveness of the patient to treatment with LA-419. In some embodiments, efficacy of LA-419 can be predicted based on the patient's genotype. Specifically, embodiments relate to determining the genotype for an individual at the NOS3 gene (e.g., determining if the subject is homozygous wildtype at position 894).

Pharmacogenomics allows a clinician or physician to target prophylactic or therapeutic treatments to individuals who will most benefit from the treatment and to avoid treatment of individuals who will experience symptomatic side effects. Thus, a physician or clinician may consider applying knowledge obtained in relevant pharmacogenomics analysis in determining whether to administer LA-419 as well as whether to modify the dosage, regimen, and/or therapeutically effective amounts to be administered so as to attain the effect desired by the treatment. In some embodiments, a physician or clinician may alter treatment of the subject by adding an additional therapy or using an alternative therapy to treatment with LA-419.

### I. TREATMENT OF CARDIOVASCULAR DISEASES AND CONDITIONS

Treatment of cardiovascular diseases and conditions, which is generally understood to refer to diseases, conditions, or disorders involving the heart or blood vessels, includes a variety of options, such as statins, diuretics, anticoagulants, beta blockers, vasodilators, ACE inhibitors, or calcium channel blockers. Vasodilators cause a relaxation of the smooth muscle that surrounds a blood vessel. It is achieved primarily by either lowering intracellular calcium concentration or dephosphorylating myosin. One way to achieve vasodilation is to induce nitric oxide.

A. NO Donors

BiDil is a relatively new drug that has been recently approved for the treatment of heart failure in African Americans. BiDil consists of a fixed dose combination of isosorbide dinitrate (vasodilator) and hydralazine (antihypertensive). Heart failure, or end-stage cardiovascular disease, affects approximately five million Americans. Epidemiologic studies indicate a higher prevalence of risk factors for heart failure among African Americans in the United States (Burt et al., 1995). The complications associated with these diseases, such as stroke, heart, and renal failure, contribute to greater rates of mortality in this population. Recent analyses of heart failure clinical trials show that the mortality rate and the hospitalization rate for African Americans are significantly higher than for non-African Americans.

Isosorbide dinitrate is a direct NO donor and causes tolerance with extended use. Hydralazine is a diuretic with putative antioxidant activity that may contribute to reduce loss of NO through its reaction in superoxide levels. The manufacturer (Nitromed) has been allowed to claim in its approved label that BiDil works through an enhancement in the bioavailability of NO in the vessel. This mechanism may provide preferential advantages to African-American heart failure patients who suffer from a greater deficiency of NO than non-African Americans.

The clinical benefit of BiDil in African Americans with heart failure was tested in the A-HeFT trial, a double-blind, placebo controlled study with 1,050 patients at 169 sites in the United States. The trial was halted in July 2004 on the recommendation of the independent Data and Safety Monitoring Board and the steering committee for the trial due to significant survival benefit seen in patients taking the drug. The results of the BiDil trial were presented at the annual meeting of the American Heart Association on Nov. 8, 2004. The trial enrolled a total of 1,050 African American patients who had New York Heart Association class III or IV heart failure with dilated ventricles, constituting moderately severe and severe levels of heart failure. The primary end point for the trial was death from any cause, hospitalization for heart failure, and change in the quality of life. The A-HeFT study was terminated early owing to a significantly higher mortality rate in the placebo group than in the treatment group. A 10.2 percent death rate was shown in the placebo group compared to a 6.2 percent death rate in the BiDil group, p=0.02. The mean composite score for the primary endpoint was significantly better in the group given BiDil than in the placebo group, p=0.01, as were its individual components: a 43 percent reduction in the rate of death from any cause, p=0.01; a 33 percent relative reduction in the rate of first hospitalization for heart failure, p=0.0001; and a statistically significant improvement in quality of life, p=0.02, measured by the Minnesota Living with Heart Failure questionnaire. Adverse events reported in the trial included symptoms of headache and dizziness, which were significantly more frequent in the group given BiDil, and exacerbations of congestive heart failure (both moderate and severe), which were significantly more frequent in the placebo group.

Other drugs that have been reported to have NO donor activity include: GEA 3162; 1,1-diethyl-2-hydroxy-2-nitrosohydrazine; diethylenetriamine; Molsidomine; isosorbide-5-mononitrate; S-Nitrosothiols; diethylamine dinitric oxide adduct; S-nitrosomercaptoethanol; 3-morpholino-sydnonimine; S-nitrosocysteine; spermine nitric oxide complex; NOC 18; 2,2′-(hydroxynitrosohydrazono)bis-ethanamine; S-Nitroso-N-Acetylpenicillamine; S-Nitrosoglutathione; S-nitro-N-acetylpenicillamine; PAPA NONOate; 3-(2-hydroxy-1-methyl-2-nitrosohydrazino)-N-methyl-1-propanamine; Nitroprusside; Isosorbide Dinitrate; FK 409.

B. LA-419

The drug S-(6-Nitro-oxi-hexahydro-furo[3,2-b]furan-3-1-il)thioacetate, which is also known as LA-419, is a nitric oxide donor that is currently in clinical trials as a treatment for cardiovascular disorders. It has also been reported as a candidate therapy for the treatment of glaucoma and intestinal disorders (Megson et al., 2009). LA-419 has the following chemical structure:

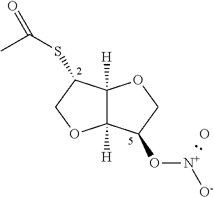

### II. DETECTION OF POLYMORPHISMS

The polymorphism described herein is present at position 894 in the NOS3 gene. The presence of the polymorphism can be determined from the sequence of the gene or by using specific characteristics of the polymorphism, e.g., restriction enzyme recognition site. As a result, a variety of different methodologies can be employed for the purpose of detecting polymorphisms in the NOS3 gene. Alternatively, the protein gene product can be evaluated to determine the patient's genotype.

A. Nucleic Acids

Certain embodiments concern various nucleic acids, including amplification primers, oligonucleotide probes, and other nucleic acid elements involved in the analysis of genomic DNA. In certain aspects, a nucleic acid comprises a wild-type, a mutant, or a polymorphic nucleic acid.

The terms “NOS3” polymorphism refer to a polymorphism in the NOS3 gene. A wildtype sequence of the NOS3 coding region is provided as SEQ ID NO:1; this sequence has a G at position 894. The sequence of the NOS3 with a polymorphism at position 894 (a T instead of a G) is shown as SEQ ID NO:3. The Genbank accession number NM_{—}000603.4, which is hereby incorporated by reference, shows a T at position 894. Position 894 corresponds to nucleotide number 1187 in SEQ ID NO:1 and SEQ ID NO:3. It is contemplated by the inventors and understood by those of skill in the art that a patient's genotype may include other polymorphisms beside the polymorphism at position 894 (1187 from the first position of the cDNA sequence). These variations in sequence may be accommodated insofar as primers and probes are designed to detect the sequence at position 894. One of ordinary skill in the art would know how to identify these polymorphisms, which are readily known, for example, in the NCBI database under the NOS3 accession number on the World Wide Web at ncbi.nlm.nih.gov/SNP/snp_ref.cgi?showRare=on&chooseRs=coding&locusId=4846&mrna=NM_{—}000603.3&ctg=NT_{—}007914.14&prot=NP_{—}000594.2&orien=forward&refresh=refresh, which is hereby incorporated by reference. It appears that 43 SNPs are currently identified in NOS3.

The location of a polymorphism can be designated based on the total number of nucleotides in the sequence starting as nucleotide 1 (which coincides with the first nucleotide encoding the first amino acid) and progressing in increments of one to the end of the sequence, i.e., nucleotide 894 of SEQ ID NO:1 and SEQ ID NO:3. The polypeptide sequence encoded by the wild-type 894 sequence is shown in SEQ ID NO:2; it has a Glu at amino acid 298. The polymorphic 894 sequence having a T encodes what is shown in SEQ ID NO:4; it has an Asp at amino acid 298.

In some embodiments, nucleic acids used in embodiments comprise or are complementary to 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1165, 1200, 1300, 1400, 1500, 1840, 1870 or more contiguous nucleotides, or any range derivable therein, of the human NOS3 sequence provided in SEQ ID NO:1 or SEQ ID NO:3, or any other sequence provided herein. One of skill in the art knows how to design and use primers and probes for hybridization and amplification of a sequence in the NOS3 gene. In some embodiments, the sequence is the NOS3 coding sequence (or its complement) or it is based on the NOS3 transcript, such as a cDNA of this sequence.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss”, a double stranded nucleic acid by the prefix “ds”, and a triple stranded nucleic acid by the prefix “ts.”

1. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in European Patent 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. In certain aspects amplification oligonucleotides can be designed on either side or overlapping with the boundaries of the insertion site. In a further aspect an oligonucleotide specific for the sequence at 894, whether a G or a T, can be designed. These oligonucleotides can varying in length from 5, 10, 15, 16, 17, 18, 19, 20, 21, 22, 23, 24, 25, 26, 27, 28, 29, 30, 50, nucleotides or more, including all values and ranges there between. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

2. Purification of Nucleic Acids

A nucleic acid may be purified on polyacrylamide gels, cesium chloride centrifugation gradients, chromatography columns or by any other means known to one of ordinary skill in the art (see for example, Sambrook et al., 2001, incorporated herein by reference).

In certain aspects, embodiments concern a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

3. Nucleic Acid Segments

In certain embodiments, the nucleic acid is a nucleic acid segment. As used herein, the term “nucleic acid segment,” are fragments of a nucleic acid, such as, for a non-limiting example, those that encode only part of a NOS3 sequence, or part of the NOS3 gene locus or gene sequence. Thus, a “nucleic acid segment” may comprise any part of a gene sequence, including from about 2 nucleotides to the full length gene including promoter regions to the polyadenylation signal and any length that includes all the coding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

4. Nucleic Acid Complements

Embodiments also encompass a nucleic acid that is complementary to a nucleic acid. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule. In preferred embodiments, a complement is a hybridization probe or amplification primer for the detection of a nucleic acid polymorphism.

As used herein, the term “complementary” or “complement” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. However, in some diagnostic or detection embodiments, completely complementary nucleic acids are used.

5. Nucleic Acid Detection and Evaluation

Genotyping can be performed using methods described in Small et al. (2002), which is incorporated herein by reference. It will be understood by the skilled artisan that other standard techniques are available for genotyping and any technique may be used with the embodiments described herein. General methods of nucleic acid detection methods are provided below.

In some embodiments, genotyping involves isolating from the patient a nucleic acid mixture comprising both copies of the NOS3 gene, or a fragment thereof, and determining the nucleotide sequence at position 894 of the NOS3 gene. In some embodiments, this involves determining the sequence based on the transcripts produced from both copies of the gene. Other polymorphisms, such as single nucleotide polymorphisms can be linked to and indicative of the polymorphism at position 894 described herein. Consequently, in some embodiments a polymorphism in linkage disequilibrium (LED or LD) with the polymorphism at position 894 may be used to determine the sequence at position 894.

Those in the art will readily recognize that nucleic acid molecules may be double-stranded molecules and that reference to a particular site on one strand refers, as well, to the corresponding site on a complementary strand. Thus, in defining a polymorphic site, reference to a sequence including an adenine, a thymine (uridine), a cytosine, or a guanine at a particular site on one strand of a nucleic acid molecule is also intended to include the thymine (uridine), adenine, guanine, or cytosine (respectively) at the corresponding site on a complementary strand of a nucleic acid molecule. Thus, reference may be made to either strand and still comprise the same polymorphic site and an oligonucleotide may be designed to hybridize to either strand. Throughout the text, in identifying a polymorphic site, reference is made to SEQ ID NO:1 or SEQ ID NO:3 for the purpose of convenience.

Typically, the nucleic acid mixture is isolated from a biological sample taken from the individual, such as a blood sample or tissue sample using standard techniques such as disclosed in Jones (1963) which is hereby incorporated by reference. Suitable tissue samples include whole blood, semen, saliva, tears, urine, fecal material, sweat, buccal, skin, and hair. The nucleic acid mixture may be comprised of genomic DNA.

The ability to predict a patient's response to LA-419 assists the treating physician in making decisions about how to treat a patient having a cardiovascular disease or condition. A patient whose genotype indicates a likelihood that the patient will respond well to LA-419 (i.e., a patient homozygous wildtype at 894 in the NOS3 gene) would be a better candidate for therapy, and the physician may treat the patient with an alternate form of therapy.

In the genotyping methods used in embodiments, the identity of a polymorphic site may be determined by amplifying a target region containing the polymorphic site directly from one or both copies of the NOS3 gene present in the individual and the sequence of the amplified region(s) determined by conventional methods or evaluated directly.

The target region(s) may be amplified using any oligonucleotide-directed amplification method, including but not limited to polymerase chain reaction (PCR) (U.S. Pat. No. 4,965,188), ligase chain reaction (LCR) (Barany et al., 1991; WO90/01069), and oligonucleotide ligation assay (OLA) (Landegren et al., 1988). Oligonucleotides useful as primers or probes in such methods should specifically hybridize to a region of the nucleic acid that contains or is adjacent to the polymorphic site. Typically, the oligonucleotides are between 10 and 35 nucleotides in length and preferably, between 15 and 30 nucleotides in length. Most preferably, the oligonucleotides are 20 to 25 nucleotides long. The exact length of the oligonucleotide will depend on many factors that are routinely considered and practiced by the skilled artisan.

Other known nucleic acid amplification procedures may be used to amplify the target region including transcription-based amplification systems (U.S. Pat. No. 5,130,238; EP 329,822; U.S. Pat. No. 5,169,766, WO89/06700) and isothermal methods (Walker et al., 1992).

A polymorphism in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

The genotype for one or more polymorphic sites in the NOS3 gene or other sites in LD with position 894 of an individual may also be determined by hybridization of one or both copies of the gene, or a fragment thereof, to nucleic acid arrays and subarrays such as described in WO 95/11995. The arrays would contain a battery of allele-specific oligonucleotides representing each of the polymorphic sites to be included in the genotype or haplotype.

The identity of polymorphisms may also be determined using a mismatch detection technique, including but not limited to the RNase protection method using riboprobes (Winter et al., 1985; Meyers et al., 1985) and proteins which recognize nucleotide mismatches, such as the E. coli mutS protein (Modrich, 1991). Alternatively, variant alleles can be identified by single strand conformation polymorphism (SSCP) analysis (Orita et al., 1989; Humphries et al., 1996) or denaturing gradient gel electrophoresis (DGGE) (Wartell et al., 1990; Sheffield et al., 1989).

A polymerase-mediated primer extension method may also be used to identify the polymorphism(s). Several such methods have been described in the patent and scientific literature. Extended primers containing a polymorphism may be detected by mass spectrometry as described in U.S. Pat. No. 5,605,798. An other primer extension method is allele-specific PCR (Ruano et al., 1989; Ruano et al., 1991; WO 93/22456; Turki et al., 1995).

Polymorphic variation in the human NOS3 gene can also be detected using differential digestion of DNA by certain restriction enzymes (Small et al., 2002) or by any other method that identifies the sequence of the polymorphic position in the NOS3 gene.

a. Hybridization

The use of a probe or primer of between 7, 8, 9, 10, 11, 12, 13, 14, 15, 16, 17, 18, 19, 20, 21, 22, 23, 24, 25, 50, 60, 70, 80, 90, or 100 consecutive nucleotides of SEQ ID NO:1 or SEQ ID NO:3, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting a specific polymorphism. It is generally appreciated that conditions can be rendered more stringent by the addition of increasing amounts of formamide. For example, under highly stringent conditions, hybridization to filter-bound DNA may be carried out in 0.5 M NaHPO₄, 7% sodium dodecyl sulfate (SDS), 1 mM EDTA at 65° C., and washing in 0.1×SSC/0.1% SDS at 68° C. (Ausubel et al., 1989).

Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Under low stringent conditions, such as moderately stringent conditions the washing may be carried out for example in 0.2×SSC/0.1% SDS at 42° C. (Ausubel et al., 1989). Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples. In other aspects, a particular nuclease cleavage site may be present and detection of a particular nucleotide sequence can be determined by the presence or absence of nucleic acid cleavage.

In general, it is envisioned that the probes or primers described herein will be useful as reagents in solution hybridization, as in PCR, for detection of expression or genotype of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

b. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples with or without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to the NOS3 gene locus, or variants thereof, and fragments thereof are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids that contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected, analyzed or quantified. In certain applications, the detection may be performed by visual means. In certain applications, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA) (described in further detail below), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, Great Britain Application 2 202 328, and in PCT Application PCT/US89/01025, each of which is incorporated herein by reference in its entirety. Qbeta Replicase, described in PCT Application PCT/US87/00880, may also be used as an amplification method in the present invention.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

c. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by spin columns and/or chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized, with or without separation. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

d. Other Assays

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

e. Specific Examples of Polymorphism Nucleic Acid Screening Methods

Spontaneous mutations that arise during the course of evolution in the genomes of organisms are often not immediately transmitted throughout all of the members of the species, thereby creating polymorphic alleles that co-exist in the species populations. Often polymorphisms are the cause of genetic diseases. Several classes of polymorphisms have been identified. For example, variable nucleotide type polymorphisms (VNTRs), arise from spontaneous tandem duplications of di- or trinucleotide repeated motifs of nucleotides. If such variations alter the lengths of DNA fragments generated by restriction endonuclease cleavage, the variations are referred to as restriction fragment length polymorphisms (RFLPs). RFLPs are been widely used in human and animal genetic analyses.

Another class of polymorphisms are generated by the replacement of a single nucleotide. Such single nucleotide polymorphisms (SNPs) rarely result in changes in a restriction endonuclease site. Thus, SNPs are rarely detectable restriction fragment length analysis. SNPs are the most common genetic variations and occur once every 100 to 300 bases and several SNP mutations have been found that affect a single nucleotide in a protein-encoding gene in a manner sufficient to actually cause a genetic disease. SNP diseases are exemplified by hemophilia, sickle-cell anemia, hereditary hemochromatosis, late-onset alzheimer disease etc.

Several methods have been developed to screen polymorphisms and some examples are listed below. The reference of Kwok and Chen (2003) and Kwok (2001) provide overviews of some of these methods; both of these references are specifically incorporated by reference.

SNPs relating to ABCC2 can be characterized by the use of any of these methods or suitable modification thereof. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or any other biochemical interpretation.

(1) DNA Sequencing

The most commonly used method of characterizing a polymorphism is direct DNA sequencing of the genetic locus that flanks and includes the polymorphism. Such analysis can be accomplished using either the “dideoxy-mediated chain termination method,” also known as the “Sanger Method” (Sanger et al., 1975) or the “chemical degradation method,” also known as the “Maxam-Gilbert method” (Maxam et al., 1977). Sequencing in combination with genomic sequence-specific amplification technologies, such as the polymerase chain reaction may be utilized to facilitate the recovery of the desired genes (Mullis et al., 1986; European Patent Application 50,424; European Patent Application. 84,796, European Patent Application 258,017, European Patent Application. 237,362; European Patent Application. 201,184; U.S. Pat. Nos. 4,683,202; 4,582,788; and 4,683,194), all of the above incorporated herein by reference.

(2) Exonuclease Resistance

Other methods that can be employed to determine the identity of a nucleotide present at a polymorphic site utilize a specialized exonuclease-resistant nucleotide derivative (U.S. Pat. No. 4,656,127). A primer complementary to an allelic sequence immediately 3′-to the polymorphic site is hybridized to the DNA under investigation. If the polymorphic site on the DNA contains a nucleotide that is complementary to the particular exonucleotide-resistant nucleotide derivative present, then that derivative will be incorporated by a polymerase onto the end of the hybridized primer. Such incorporation makes the primer resistant to exonuclease cleavage and thereby permits its detection. As the identity of the exonucleotide-resistant derivative is known one can determine the specific nucleotide present in the polymorphic site of the DNA.

(3) Microsequencing Methods

Several other primer-guided nucleotide incorporation procedures for assaying polymorphic sites in DNA have been described (Komher et al., 1989; Sokolov, 1990; Syvanen 1990; Kuppuswamy et al., 1991; Prezant et al., 1992; Ugozzoll et al., 1992; Nyren et al., 1993). These methods rely on the incorporation of labeled deoxynucleotides to discriminate between bases at a polymorphic site. As the signal is proportional to the number of deoxynucleotides incorporated, polymorphisms that occur in runs of the same nucleotide result in a signal that is proportional to the length of the run (Syvanen et al., 1990).

(4) Extension in Solution

French Patent 2,650,840 and PCT Application WO91/02087 discuss a solution-based method for determining the identity of the nucleotide of a polymorphic site. According to these methods, a primer complementary to allelic sequences immediately 3′-to a polymorphic site is used. The identity of the nucleotide of that site is determined using labeled dideoxynucleotide derivatives which are incorporated at the end of the primer if complementary to the nucleotide of the polymorphic site.

(5) Genetic Bit Analysis or Solid-Phase Extension

PCT Application WO92/15712 describes a method that uses mixtures of labeled terminators and a primer that is complementary to the sequence 3′ to a polymorphic site. The labeled terminator that is incorporated is complementary to the nucleotide present in the polymorphic site of the target molecule being evaluated and is thus identified. Here the primer or the target molecule is immobilized to a solid phase.

(6) Oligonucleotide Ligation Assay (OLA)

This is another solid phase method that uses different methodology (Landegren et al., 1988). Two oligonucleotides, capable of hybridizing to abutting sequences of a single strand of a target DNA are used. One of these oligonucleotides is biotinylated while the other is detectably labeled. If the precise complementary sequence is found in a target molecule, the oligonucleotides will hybridize such that their termini abut, and create a ligation substrate. Ligation permits the recovery of the labeled oligonucleotide by using avidin. Other nucleic acid detection assays, based on this method, combined with PCR have also been described (Nickerson et al., 1990). Here PCR is used to achieve the exponential amplification of target DNA, which is then detected using the OLA.

(7) Ligase/Polymerase-Mediated Genetic Bit Analysis

U.S. Pat. No. 5,952,174 describes a method that also involves two primers capable of hybridizing to abutting sequences of a target molecule. The hybridized product is formed on a solid support to which the target is immobilized. Here the hybridization occurs such that the primers are separated from one another by a space of a single nucleotide. Incubating this hybridized product in the presence of a polymerase, a ligase, and a nucleoside triphosphate mixture containing at least one deoxynucleoside triphosphate allows the ligation of any pair of abutting hybridized oligonucleotides. Addition of a ligase results in two events required to generate a signal, extension and ligation. This provides a higher specificity and lower “noise” than methods using either extension or ligation alone and unlike the polymerase-based assays, this method enhances the specificity of the polymerase step by combining it with a second hybridization and a ligation step for a signal to be attached to the solid phase.

(8) Invasive Cleavage Reactions

Invasive cleavage reactions can be used to evaluate cellular DNA for a particular polymorphism. A technology called INVADER® employs such reactions (e.g., de Arruda et al., 2002; Stevens et al., 2003, which are incorporated by reference). Generally, there are three nucleic acid molecules: 1) an oligonucleotide upstream of the target site (“upstream oligo”), 2) a probe oligonucleotide covering the target site (“probe”), and 3) a single-stranded DNA with the target site (“target”). The upstream oligo and probe do not overlap but they contain contiguous sequences. The probe contains a donor fluorophore, such as fluoroscein, and an acceptor dye, such as Dabcyl. The nucleotide at the 3′ terminal end of the upstream oligo overlaps (“invades”) the first base pair of a probe-target duplex. Then the probe is cleaved by a structure-specific 5′ nuclease causing separation of the fluorophore/quencher pair, which increases the amount of fluorescence that can be detected. See Lu et al., 2004.

In some cases, the assay is conducted on a solid-surface or in an array format.

(9) Other Methods to Detect SNPs

Several other specific methods for polymorphism detection and identification are presented below and may be used as such or with suitable modifications in conjunction with identifying polymorphisms of the NOS3 gene in the present invention. Several other methods are also described on the SNP web site of the NCBI on the World Wide Web at ncbi.nlm.nih.gov/SNP, incorporated herein by reference.

In a particular embodiment, extended haplotypes may be determined at any given locus in a population, which allows one to identify exactly which SNPs will be redundant and which will be essential in association studies. The latter is referred to as ‘haplotype tag SNPs (htSNPs)’, markers that capture the haplotypes of a gene or a region of linkage disequilibrium. See Johnson et al. (2001) and Ke and Cardon (2003), each of which is incorporated herein by reference, for exemplary methods.

The VDA-assay utilizes PCR amplification of genomic segments by long PCR methods using TaKaRa LA Taq reagents and other standard reaction conditions. The long amplification can amplify DNA sizes of about 2,000-12,000 bp. Hybridization of products to variant detector array (VDA) can be performed by a Affymetrix High Throughput Screening Center and analyzed with computerized software.

A method called Chip Assay uses PCR amplification of genomic segments by standard or long PCR protocols. Hybridization products are analyzed by VDA, Halushka et al. (1999), incorporated herein by reference. SNPs are generally classified as “Certain” or “Likely” based on computer analysis of hybridization patterns. By comparison to alternative detection methods such as nucleotide sequencing, “Certain” SNPs have been confirmed 100% of the time; and “Likely” SNPs have been confirmed 73% of the time by this method.

Other methods simply involve PCR amplification following digestion with the relevant restriction enzyme. Yet others involve sequencing of purified PCR products from known genomic regions.

In yet another method, individual exons or overlapping fragments of large exons are PCR-amplified. Primers are designed from published or database sequences and PCR-amplification of genomic DNA is performed using the following conditions: 200 ng DNA template, 0.5 μM each primer, 80 μM each of dCTP, dATP, dTTP and dGTP, 5% formamide, 1.5 mM MgCl₂, 0.5 U of Taq polymerase and 0.1 volume of the Taq buffer. Thermal cycling is performed and resulting PCR-products are analyzed by PCR-single strand conformation polymorphism (PCR-SSCP) analysis, under a variety of conditions, e.g, 5 or 10% polyacrylamide gel with 15% urea, with or without 5% glycerol. Electrophoresis is performed overnight. PCR-products that show mobility shifts are reamplified and sequenced to identify nucleotide variation.

In a method called CGAP-GAI (DEMIGLACE), sequence and alignment data (from a PHRAP.ace file), quality scores for the sequence base calls (from PHRED quality files), distance information (from PHYLIP dnadist and neighbour programs) and base-calling data (from PHRED ‘-d’ switch) are loaded into memory. Sequences are aligned and examined for each vertical chunk (‘slice’) of the resulting assembly for disagreement. Any such slice is considered a candidate SNP (DEMIGLACE). A number of filters are used by DEMIGLACE to eliminate slices that are not likely to represent true polymorphisms. These include filters that: (i) exclude sequences in any given slice from SNP consideration where neighboring sequence quality scores drop 40% or more; (ii) exclude calls in which peak amplitude is below the fifteenth percentile of all base calls for that nucleotide type; (iii) disqualify regions of a sequence having a high number of disagreements with the consensus from participating in SNP calculations; (iv) removed from consideration any base call with an alternative call in which the peak takes up 25% or more of the area of the called peak; (v) exclude variations that occur in only one read direction. PHRED quality scores were converted into probability-of-error values for each nucleotide in the slice. Standard Baysian methods are used to calculate the posterior probability that there is evidence of nucleotide heterogeneity at a given location.

In a method called CU-RDF (RESEQ), PCR amplification is performed from DNA isolated from blood using specific primers for each SNP, and after typical cleanup protocols to remove unused primers and free nucleotides, direct sequencing using the same or nested primers.

In a method called DEBNICK (METHOD-B), a comparative analysis of clustered EST sequences is performed and confirmed by fluorescent-based DNA sequencing. In a related method, called DEBNICK (METHOD-C), comparative analysis of clustered EST sequences with phred quality >20 at the site of the mismatch, average phred quality >=20 over 5 bases 5′-FLANK and 3′ to the SNP, no mismatches in 5 bases 5′ and 3′ to the SNP, at least two occurrences of each allele is performed and confirmed by examining traces.

In a method identified by ERO (RESEQ), new primers sets are designed for electronically published STSs and used to amplify DNA from 10 different mouse strains. The amplification product from each strain is then gel purified and sequenced using a standard dideoxy, cycle sequencing technique with ³³P-labeled terminators. All the ddATP terminated reactions are then loaded in adjacent lanes of a sequencing gel followed by all of the ddGTP reactions and so on. SNPs are identified by visually scanning the radiographs.

In another method identified as ERO (RESEQ-HT), new primers sets are designed for electronically published murine DNA sequences and used to amplify DNA from 10 different mouse strains. The amplification product from each strain is prepared for sequencing by treating with Exonuclease I and Shrimp Alkaline Phosphatase. Sequencing is performed using ABI Prism Big Dye Terminator Ready Reaction Kit (Perkin-Elmer) and sequence samples are run on the 3700 DNA Analyzer (96 Capillary Sequencer).

FGU-CBT (SCA2-SNP) identifies a method where the region containing the SNP were PCR amplified using the primers SCA2-FP3 and SCA2-RP3. Approximately 100 ng of genomic DNA is amplified in a 50 ml reaction volume containing a final concentration of 5 mM Tris, 25 mM KCl, 0.75 mM MgCl₂, 0.05% gelatin, 20 pmol of each primer and 0.5 U of Taq DNA polymerase. Samples are denatured, annealed and extended and the PCR product is purified from a band cut out of the agarose gel using, for example, the QIAquick gel extraction kit (Qiagen) and is sequenced using dye terminator chemistry on an ABI Prism 377 automated DNA sequencer with the PCR primers.

In a method identified as JBLACK (SEQ/RESTRICT), two independent PCR reactions are performed with genomic DNA. Products from the first reaction are analyzed by sequencing, indicating a unique FspI restriction site. The mutation is confirmed in the product of the second PCR reaction by digesting with Fsp I.

In a method described as KWOK(1), SNPs are identified by comparing high quality genomic, sequence data from four randomly chosen individuals by direct DNA sequencing of PCR products with dye-terminator chemistry (see Kwok et al., 1996). In a related method identified as KWOK(2) SNPs are identified by comparing high quality genomic sequence data from overlapping large-insert clones such as bacterial artificial chromosomes (BACs) or P1-based artificial chromosomes (PACs). An STS containing this SNP is then developed and the existence of the SNP in various populations is confirmed by pooled DNA sequencing (see Taillon-Miller et al., 1998). In another similar method called KWOK(3), SNPs are identified by comparing high quality genomic sequence data from overlapping large-insert clones BACs or PACs. The SNPs found by this approach represent DNA sequence variations between the two donor chromosomes but the allele frequencies in the general population have not yet been determined. In method KWOK(5), SNPs are identified by comparing high quality genomic sequence data from a homozygous DNA sample and one or more pooled DNA samples by direct DNA sequencing of PCR products with dye-terminator chemistry. The STSs used are developed from sequence data found in publicly available databases. Specifically, these STSs are amplified by PCR against a complete hydatidiform mole (CHM) that has been shown to be homozygous at all loci and a pool of DNA samples from 80 CEPH parents (see Kwok et al., 1994).

In another such method, KWOK (OverlapSnpDetectionWithPolyBayes), SNPs are discovered by automated computer analysis of overlapping regions of large-insert human genomic clone sequences. For data acquisition, clone sequences are obtained directly from large-scale sequencing centers. This is necessary because base quality sequences are not present/available through GenBank. Raw data processing involves analyzed of clone sequences and accompanying base quality information for consistency. Finished (‘base perfect’, error rate lower than 1 in 10,000 bp) sequences with no associated base quality sequences are assigned a uniform base quality value of 40 (1 in 10,000 bp error rate). Draft sequences without base quality values are rejected. Processed sequences are entered into a local database. A version of each sequence with known human repeats masked is also stored. Repeat masking is performed with the program “MASKERAID.” Overlap detection: Putative overlaps are detected with the program “WUBLAST.” Several filtering steps' followed in order to eliminate false overlap detection results, i.e. similarities between a pair of clone sequences that arise due to sequence duplication as opposed to true overlap. Total length of overlap, overall percent similarity, number of sequence differences between nucleotides with high base quality value “high-quality mismatches.” Results are also compared to results of restriction fragment mapping of genomic clones at Washington University Genome Sequencing Center, finisher's reports on overlaps, and results of the sequence contig building effort at the NCBI. SNP detection: Overlapping pairs of clone sequence are analyzed for candidate SNP sites with the ‘POLYBAYES’ SNP detection software. Sequence differences between the pair of sequences are scored for the probability of representing true sequence variation as opposed to sequencing error. This process requires the presence of base quality values for both sequences. High-scoring candidates are extracted. The search is restricted to substitution-type single base pair variations. Confidence score of candidate SNP is computed by the POLYBAYES software.

In method identified by KWOK (TaqMan assay), the TaqMan assay is used to determine genotypes for 90 random individuals. In method identified by KYUGEN(Q1), DNA samples of indicated populations are pooled and analyzed by PLACE-SSCP. Peak heights of each allele in the pooled analysis are corrected by those in a heterozygote, and are subsequently used for calculation of allele frequencies. Allele frequencies higher than 10% are reliably quantified by this method. Allele frequency=0 (zero) means that the allele was found among individuals, but the corresponding peak is not seen in the examination of pool. Allele frequency=0-0.1 indicates that minor alleles are detected in the pool but the peaks are too low to reliably quantify.

In yet another method identified as KYUGEN (Method1), PCR products are post-labeled with fluorescent dyes and analyzed by an automated capillary electrophoresis system under SSCP conditions (PLACE-SSCP). Four or more individual DNAs are analyzed with or without two pooled DNA (Japanese pool and CEPH parents pool) in a series of experiments. Alleles are identified by visual inspection. Individual DNAs with different genotypes are sequenced and SNPs identified. Allele frequencies are estimated from peak heights in the pooled samples after correction of signal bias using peak heights in heterozygotes. For the PCR primers are tagged to have 5′-ATT or 5′-GTT at their ends for post-labeling of both strands. Samples of DNA (10 ng/ul) are amplified in reaction mixtures containing the buffer (10 mM Tris-HCl, pH 8.3 or 9.3, 50 mM KCl, 2.0 mM MgCl₂), 0.25 μM of each primer, 200 μM of each dNTP, and 0.025 units/μl of Taq DNA polymerase premixed with anti-Taq antibody. The two strands of PCR products are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. For the SSCP: an aliquot of fluorescently labeled PCR products and TAMRA-labeled internal markers are added to deionized formamide, and denatured. Electrophoresis is performed in a capillary using an ABI Prism 310 Genetic Analyzer. Genescan softwares (P-E Biosystems) are used for data collection and data processing. DNA of individuals (two to eleven) including those who showed different genotypes on SSCP are subjected for direct sequencing using big-dye terminator chemistry, on ABI Prism 310 sequencers. Multiple sequence trace files obtained from ABI Prism 310 are processed and aligned by Phred/Phrap and viewed using Consed viewer. SNPs are identified by PolyPhred software and visual inspection.

In yet another method identified as KYUGEN (Method2), individuals with different genotypes are searched by denaturing HPLC (DHPLC) or PLACE-SSCP (Inazuka et al., 1997) and their sequences are determined to identify SNPs. PCR is performed with primers tagged with 5′-ATT or 5′-GTT at their ends for post-labeling of both strands. DHPLC analysis is carried out using the WAVE DNA fragment analysis system (Transgenomic). PCR products are injected into DNASep column, and separated under the conditions determined using WAVEMaker program (Transgenomic). The two strands of PCR products that are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. SSCP followed by electrophoresis is performed in a capillary using an ABI Prism 310 Genetic Analyzer. Genescan softwares (P-E Biosystems). DNA of individuals including those who showed different genotypes on DHPLC or SSCP are subjected for direct sequencing using big-dye terminator chemistry, on ABI Prism 310 sequencer. Multiple sequence trace files obtained from ABI Prism 310 are processed and aligned by Phred/Phrap and viewed using Consed viewer. SNPs are identified by PolyPhred software and visual inspection. Trace chromatogram data of EST sequences in Unigene are processed with PHRED. To identify likely SNPs, single base mismatches are reported from multiple sequence alignments produced by the programs PHRAP, BRO and POA for each Unigene cluster. BRO corrected possible misreported EST orientations, while POA identified and analyzed non-linear alignment structures indicative of gene mixing/chimeras that might produce spurious SNPs. Bayesian inference is used to weigh evidence for true polymorphism versus sequencing error, misalignment or ambiguity, misclustering or chimeric EST sequences, assessing data such as raw chromatogram height, sharpness, overlap and spacing; sequencing error rates; context-sensitivity; cDNA library origin, etc.

In method identified as MARSHFIELD (Method-B), overlapping human DNA sequences which contained putative insertion/deletion polymorphisms are identified through searches of public databases. PCR primers which flanked each polymorphic site are selected from the consensus sequences. Primers are used to amplify individual or pooled human genomic DNA. Resulting PCR products are resolved on a denaturing polyacrylamide gel and a PhosphorImager is used to estimate allele frequencies from DNA pools.

f. Linkage Disequilibrium

Polymorphisms in linkage disequilibrium with another polymorphism in which identification of one polymorphism is predictive of the identity of the linked polymorphism. “Linkage disequilibrium” (“LD” as used herein, though also referred to as “LED” in the art) refers to a situation where a particular combination of alleles (i.e., a variant form of a given gene) or polymorphisms at two loci appears more frequently than would be expected by chance. “Significant” as used in respect to linkage disequilibrium, as determined by one of skill in the art, is contemplated to be a statistical p or α value that may be 0.25 or 0.1 and may be 0.1, 0.05. 0.001, 0.00001 or less. The polymorphism at position 874 in the NOS3 may be determined by evaluating the nucleic acid sequence of a polymorphism in linkage disequilibrium with the 874 polymorphism. The invention may be implemented in this manner with respect to one or more polymorphisms so as to allow haplotype analysis. “Haplotype” is used according to its plain and ordinary meaning to one skilled in the art. It refers to a collective genotype of two or more alleles or polymorphisms along one of the homologous chromosomes.

The term “polymorphism”, as used herein, refers to a difference in the nucleotide or amino acid sequence of a given nucleotide or amino acid region as compared to a nucleotide or amino acid sequence in the corresponding region of another individual of the same species. Preferably, the species is human. A polymorphism is generally defined in relation to a “reference” sequence. In the subject application, “reference” sequence and “wild type” sequence are used interchangeably. Nucleotide polymorphisms include single nucleotide differences, differences in sequence of more than one nucleotide, and single or multiple nucleotide insertions, inversions, substitutions, and deletions. Amino acid polymorphisms include single amino acid differences, differences in sequence of more than one amino acid, and single or multiple amino acid insertions, substitutions, and deletions.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term biological sample encompasses a clinical sample, and also includes cells in culture, cell supernatants, cell lysates, serum, plasma, biological fluid, and tissue samples. In one embodiment, the sample is collected by the individual. For example, an individual can collect a swap of tissue from the inside of the cheek for use as a nucleic acid sample. As known in the art, many types of samples can be used for the extraction of nucleic acids.

As used herein the term “treating” in reference to a disease or condition means a reduction in severity or elimination of one or more symptoms associated with a particular disease or condition. Therefore, treating a disorder does not necessarily mean a reduction in severity of all symptoms associated with a disorder and does not necessarily mean a complete reduction in the severity of one or more symptoms associated with a disorder. Treatment, as used in this context, covers any treatment of a symptomatic condition, such as an adverse reaction in a mammal, particularly in a human, and includes: (a) diagnosing and then preventing the adverse reaction from occurring in an individual which can be predisposed to the reaction but has not yet been diagnosed as having it; (b) inhibiting the adverse reaction, i.e., arresting its development; and (c) relieving the adverse reaction, i.e., causing regression of the reaction.

The term “therapeutically effective amount” means an amount that is effective in treating a particular disorder; that is an amount that is effective for reducing the severity of one or more symptoms associated with the particular disease or condition for which treatment is sought. The term “ameliorate,” as used for instance in the amelioration of a particular condition means to make one or more symptoms of the condition at least more tolerable, if not better. The term ameliorate does not necessarily mean an increase in toleration of all symptoms associated with a disorder and does not necessarily mean a complete reduction in the severity of one or more symptoms associated with a disorder.

In another embodiment, a further step is added wherein a portion of the NOS3 gene of SEQ ID NO:1 and/or SEQ ID NO:3 is amplified prior to the identifying step. In another embodiment, the identifying is performed by a method selected from the group consisting of a hybridization assay, a sequencing assay, a microsequencing assay, a MALDI-TOF assay, and an allele-specific amplification assay. In a further embodiment, the identifying is performed by an antibody-based assay.

Pharmacogenomics allows a clinician or physician to target prophylactic or therapeutic treatments to individuals who will most benefit from the treatment and to avoid treatment of individuals who will experience symptomatic side effects, in the case of LA-419 the adverse side effect can be tolerance and toxicity to the drug. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer LA-419 as well as tailoring the dosage, regimen, and/or therapeutically effective amounts to be administered so as to attain the effect desired by treatment with the modulator.

A determination of how a given NOS3 polymorphism is predictive of an individual's likelihood of responding to an NO donor can be accomplished by determining the genotype of the individual in the NOS3 gene, as described herein. Information generated from one or more of these approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating an individual with a niacin receptor modulator, such as niacin or an analog thereof.

In some embodiments, the difference in protein sequence can provide a basis for determining the nucleotide sequence at position 894. The wildtype NOS3 sequence encodes a Glu at position 298, in contrast to the Asp at that position when there is a T ant position 894. One of skill in the art appreciates that reagents and assays are available to detect the different proteins. In some embodiments, a method or kit involves an antibody that recognizes both forms of the protein or only one form. The antibody may be a monoclonal antibody or a polyclonal antibody.

In one embodiment there are kits for use in the methods of the invention, for example, a kit for determining a level of probability for an individual for a condition responsiveness to LA-419 therapy, a kit for using a NOS3 zygosity of an individual for determining a suitability or an unsuitability of an individual for inclusion in a clinical trial, or a kit for determining a level of probability for a condition associated with heart failure. A kit can comprise reagents and instructions for performing the methods described herein. For example, a kit can include genotyping reagents such as reagents for isolating nucleic acid molecules and reagents for amplifying nucleic acid molecules such as primers. A kit can also include, for example, a NOS3 assay such as an ELISA. In addition, a kit can contain control samples, for example, to show that amplification reactions are not contaminated.

The contents of the kit are contained in packaging material, preferably to provide a sterile, contaminant-free environment. In addition, the packaging material contains instructions indicating how the materials within the kit can be employed. The instructions for use typically include a tangible expression describing the reagent concentration or at least one assay method parameter, such as the relative amounts of reagent and sample to be admixed, maintenance time periods for reagent/sample admixtures, temperature, buffer conditions, and the like.

### III. METHODS OF TREATING CARDIAC HYPERTROPHY

Once the NOS3 genotype of the individual is determined a therapeutic course of treatment may be individualized. In an embodiment of the method, the trait of interest is a clinical response exhibited by a patient to some therapeutic treatment, for example, response to a NO donor drug such as, but not limited to, a LA-419. The term “clinical response” means a quantitative measure of the efficacy or potency of the therapy and adverse events (i.e., side effects).

Thus, individuals homozygous for an insertional polymorphism in the NOS3 gene having or suspected of having or at risk of developing heart failure can be placed on a therapy that includes NO donors such as, but not limited to, LA-419. The NO donor may be administered alone or in combination with at least one other agent, such as a stabilizing compound.

A. Routes of Administration

Administration of the NO donor may be by any number of routes including, but not limited to oral, intravenous, intramuscular, intra-arterial, intramedullary, intrathecal, intraventricular, intradermal, intratracheal, intravesicle, intraocular, transdermal, subcutaneous, intraperitoneal, intranasal, enteral, topical, sublingual, or rectal. Further details on techniques for formulation and administration may be found in the latest edition of Remington's Pharmaceutical Sciences (Maack Publishing Co., Easton, Pa.). In certain embodiments LA-419 or other NO donors are formulated for oral administration.

B. Formulations

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers. Aqueous compositions comprise an effective amount of the drug, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

The active compositions of the present invention may include classic pharmaceutical preparations. Administration of these compositions according to the present invention may be via any common route so long as the target tissue is available via that route. This includes oral, nasal, or buccal. Alternatively, administration may be by intradermal, intraarterial, subcutaneous, intramuscular, intraperitoneal or intravenous injection, or by direct injection into cardiac tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

The compositions of the present invention generally may be formulated in a neutral or salt form. Pharmaceutically-acceptable, salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

1. Tablets and Capsules

Tablet or capsule formulations are well known to those of skill in the art. In some embodiments the drug as in disclosed in U.S. Pat. No. 5,126,145, which is incorporated by reference herein. In one embodiment, a tablet comprises, in admixture, about 5-30% high viscosity hydroxypropyl methyl cellulose, about 2-15% of a water-soluble pharmaceutical binder, about 2-20% of a hydrophobic component such as a waxy material, e.g., a fatty acid, and about 30-90% active ingredient.

2. Films

In some embodiments methods for preventing or treating a patient having a homozygous wildtype polymorphism in the NOS3 gene following an invasive cardiac procedure comprising administering biodegradable, biocompatible polymeric film comprising a NO donor, such as LA-419, to a patient. The polymeric films are thin compared to their length and breadth. The films typically have a uniform selected thickness between about 60 micrometers and about 5 mm. Films of between about 600 micrometers and 1 mm and between about 1 mm and about 5 mm thick, as well as films between about 60 micrometers and about 1000 micrometers, and between about 60 and about 300 micrometers are useful in the manufacture of therapeutic implants for insertion into a patient's body. The films can be administered to the patient in a manner similar to methods used in adhesion surgeries. For example, an NO donor film formulation can be sprayed or dropped onto a cardiac tissue site or artery during surgery, or a formed film can be placed over the selected tissue site. In an alternative embodiment, the film can be used as controlled release coating on a medical device such as a stent, as is discussed in further detail below.

Either biodegradable or nonbiodegradable polymers may be used to fabricate implants in which the NO donor is uniformly distributed throughout the polymer matrix. A number of suitable biodegradable polymers for use in making the biodegradable films of this invention are known to the art, including polyanhydrides and aliphatic polyesters, preferably polylactic acid (PLA), polyglycolic acid (PGA) and mixtures and copolymers thereof, more preferably 50:50 copolymers of PLA:PGA and most preferably 75:25 copolymers of PLA:PGA. Single enantiomers of PLA may also be used, preferably L-PLA, either alone or in combination with PGA. Polycarbonates, polyfumarates and caprolactones may also be used to make the implants of this invention.

The amount of the NO donor to be incorporated into the polymeric films of this invention is an amount effective to show a measurable effect in treating diseases having similar pathophysiological states, such as but not limited to heart failure. The composition of the present invention can be incorporated into the film by various techniques such as by solution methods, suspension methods, or melt pressing.

3. Transdermal Patch Device

Transdermal delivery involves delivery of a therapeutic agent through the skin for distribution within the body by circulation of the blood. Transdermal delivery can be compared to continuous, controlled intravenous delivery of a drug using the skin as a port of entry instead of an intravenous needle. The therapeutic agent passes through the outer layers of the skin, diffuses into the capillaries or tiny blood vessels in the skin and then is transported into the main circulatory system.

Transdermal patch devices that provide a controlled, continuous administration of a therapeutic agent through the skin are well known in the art. Such devices, for example, are disclosed in U.S. Pat. Nos. 4,627,429; 4,784,857; 5,662,925; 5,788,983; and 6,113,940, which are all incorporated herein by reference. Characteristically, these devices contain a drug impermeable backing layer which defines the outer surface of the device and a permeable skin attaching membrane, such as an adhesive layer, sealed to the barrier layer in such a way as to create a reservoir between them in which the therapeutic agent is placed. Some embodiments involve a formulation of the NO donor that is introduced into the reservoir of a transdermal patch and used by a patient who is homozygous wildtype at position 894 at the NOS3 gene.

4. Medical Devices

Another embodiment contemplates the incorporation of an NO donor into a medical device that is then positioned to a desired target location within the body, whereupon the NO donor elutes from the medical device. As used herein, “medical device” refers to a device that is introduced temporarily or permanently into a mammal for the prophylaxis or therapy of a medical condition. These devices include any that are introduced, subcutaneously, percutaneously or surgically to rest within an organ, tissue or lumen. Medical devices include, but are not limited to, stents, synthetic grafts, artificial heart valves, artificial hearts and fixtures to connect the prosthetic organ to the vascular circulation, venous valves, abdominal aortic aneurysm (AAA) grafts, inferior venal caval filters, catheters including permanent drug infusion catheters, embolic coils, embolic materials used in vascular embolization (e.g., PVA foams), mesh repair materials, a Dracon vascular particle orthopedic metallic plates, rods and screws and vascular sutures.

In one embodiment, the medical device such as a stent or graft is coated with a matrix. The matrix used to coat the stent or graft according to this invention may be prepared from a variety of materials. A primary requirement for the matrix is that it be sufficiently elastic and flexible to remain unruptured on the exposed surfaces of the stent or synthetic graft.

5. Controlled/Extended/Sustained/ProlongedRelease Administration

(1) Another aspect of this invention provides methods of treating heart failure patients by delivering a NO donor to a patient, having a homozygous wildtype polymorphism genotype, as a controlled release formulation. As used herein, the terms “controlled,” “extended,” “sustained,” or “prolonged” release of the composition of the present invention will collectively be referred to herein as “controlled release,” and includes continuous or discontinuous, and linear or non-linear release of the composition in various embodiments.

C. Dosages

The amount of an NO donor (e.g., LA-419) that is administered or prescribed to the patient can be about, at least about, or at most about 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500 mg, or any range derivable therein. Alternatively, the amount administered or prescribed may be about, at least about, or at most about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4:1, 4.2, 4.3, 4.4, 4.5, 4.6, 4.7, 4.8, 4.9, 5.0 mg/kg, or any range derivable therein, with respect to the weight of the patient.

When provided in a discrete amount, each intake of the NO donor can be considered a “dose.” A medical practitioner may prescribe or administer multiple doses of the NO donor over a particular time course (treatment regimen) or indefinitely.

The NO donor may be administered 1, 2, 3, 4, 5, 6, 7, 8, 9, 10, 11, 12, 13, 14, 15, 16, 17, 18, 19, 20, 30, 40, 50, 60, 70, 80, or more times or any range derivable therein. It is further contemplated that the drug may be taken for an indefinite period of time or for as long as the patient exhibits symptoms of the medical condition for which an NO donor was prescribed or administered. Also, the drug may be administered every 2, 3, 4, 5, 6, 7, 8, 9, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months or more, or any range derivable therein. Alternatively, it may be administered systemically over any such period of time and be extended beyond more than a year. In certain embodiments, the drug is administered to or by the patient once, twice, three times, four times a day, or as needed.

D. Other Therapeutic Options

In certain embodiments of the invention, methods may involve treating the patient with an NO donor that is not LA-419 or that is a diuretic, ACE-I, AII antagonist, beta-blocker, BNP, Ca⁺⁺-blocker, or an HDAC inhibitor. Alternatively, a different NO donor other than LA-419 may be used to treat the patient. These agents may be prescribed for or administered by or to the patient instead of or in addition to an NO donor after the NOS3 polymorphism(s) are evaluated.

As a second therapeutic regimen, the agent may be administered or taken at the same time as LA-419, or either before or after LA-419. The treatment may improve one or more symptoms of the cardiovascular disease or condition such as providing increased exercise capacity, increased cardiac ejection volume, decreased left ventricular end diastolic pressure, decreased pulmonary capillary wedge pressure, increased cardiac output or cardiac index, lowered pulmonary artery pressures, decreased left ventricular end systolic and diastolic dimensions, decreased left and right ventricular wall stress, decreased wall tension and wall thickness, increased quality of life, and decreased disease-related morbidity and mortality.

In another embodiment, it is envisioned to use an NO donor in combination with other therapeutic modalities. Thus, in addition to the therapies described above, one may also provide to the patient more “standard” pharmaceutical cardiac therapies. Examples of other therapies include, without limitation, beta blockers, anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists and cytokine blockers/inhibitors, and HDAC inhibitors.

Combinations may be achieved by contacting cardiac cells with a single composition or pharmacological formulation that includes both agents, or by contacting the cell with two distinct compositions or formulations, at the same time, wherein one composition includes the expression construct and the other includes the agent. Alternatively, the therapy using an NO donor such as LA-419 may precede or follow administration of the other agent(s) by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either LA-419, or the other agent will be desired. In this regard, various combinations may be employed. By way of illustration, where LA-419 is “A” and the other agent is “B”, the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated.

1. Pharmacological Therapeutic Agents

Pharmacological therapeutic agents and methods of administration, dosages, etc., are well known to those of skill in the art (see for example, the “Physicians Desk Reference”, Klaassen's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

a. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present invention, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.

(1) Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

(2) Resins/Bile Acid Sequesterants

Non-limiting examples of resins/bile acid sequesterants include cholestyramine (cholybar, questran), colestipol (colestid) and polidexide.

(3) HMG CoA Reductase Inhibitors

Non-limiting examples of HMG CoA reductase inhibitors include lovastatin (mevacor), pravastatin (pravochol) or simvastatin (zocor).

(4) Nicotinic Acid Derivatives

Non-limiting examples of nicotinic acid derivatives include nicotinate, acepimox, niceritrol, nicoclonate, nicomol and oxiniacic acid.

(5) Thyroid Hormones and Analogs

Non-limiting examples of thyroid hormones and analogs thereof include etoroxate, thyropropic acid and thyroxine.

(6) Miscellaneous Antihyperlipoproteinemics

Non-limiting examples of miscellaneous antihyperlipoproteinemics include acifran, azacosterol, benfluorex, β-benzalbutyramide, carnitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, γ-oryzanol, pantethine, pentaerythritol tetraacetate, α-phenylbutyramide, pirozadil, probucol (lorelco), β-sitosterol, sultosilic acid-piperazine salt, tiadenol, triparanol and xenbucin.

b. Antiarteriosclerotics

Non-limiting examples of an antiarteriosclerotic include pyridinol carbamate.

c. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.

(1) Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

(2) Antiplatelet Agents

Non-limiting examples of antiplatelet agents include aspirin, a dextran, dipyridamole (persantin), heparin, sulfinpyranone (anturane) and ticlopidine (ticlid).

(3) Thrombolytic Agents

Non-limiting examples of thrombolytic agents include tissue plaminogen activator (activase), plasmin, pro-urokinase, urokinase (abbokinase) streptokinase (streptase), anistreplase/APSAC (eminase).

d. Blood Coagulants

In certain embodiments wherein a patient is suffering from a hemmorage or an increased likelyhood of hemmoraging, an agent that may enhance blood coagulation may be used. Non-limiting examples of a blood coagulation promoting agent include thrombolytic agent antagonists and anticoagulant antagonists.

(1) Anticoagulant Antagonists

Non-limiting examples of anticoagulant antagonists include protamine and vitamine K1.

(2) Thrombolytic Agent Antagonists and Antithrombotics

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

e. Antiarrhythmic Agents

Non-limiting examples of antiarrhythmic agents include Class I antiarrythmic agents (sodium channel blockers), Class II antiarrythmic agents (beta-adrenergic blockers), Class II antiarrythmic agents (repolarization prolonging drugs), Class IV antiarrhythmic agents (calcium channel blockers) and miscellaneous antiarrythmic agents.

(1) Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocalne), tocamide (tonocard) and mexiletine (mexitil). Non-limiting examples of Class IC antiarrhythmic agents include encamide (enkaid) and flecamide (tambocor).

(2) Beta Blockers

Non-limiting examples of a beta blocker, otherwise known as a β-adrenergic blocker, a β-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

(3) Repolarization Prolonging Agents

Non-limiting examples of an agent that prolong repolarization, also known as a Class III antiarrhythmic agent, include amiodarone (cordarone) and sotalol (betapace).

(4) Calcium Channel Blockers/Antagonist

Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexyline. In certain embodiments a calcium channel blocker comprises a long-acting dihydropyridine (nifedipine-type) calcium antagonist.

(5) Miscellaneous Antiarrhythmic Agents

Non-limiting examples of miscellaneous antiarrhymic agents include adenosine (adenocard), digoxin (lanoxin), acecamide, ajmaline, amoproxan, aprindine, bretylium tosylate, bunaftine, butobendine, capobenic acid, cifenline, disopyranide, hydroquinidine, indecamide, ipatropium bromide, lidocaine, lorajmine, lorcamide, meobentine, moricizine, pirmenol, prajmaline, propafenone, pyrinoline, quinidine polygalacturonate, quinidine sulfate and viquidil.

f. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.

(1) Alpha Blockers

Non-limiting examples of an alpha blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

(2) Alpha/Beta Blockers

In certain embodiments, an antihypertensive agent is both an alpha and beta adrenergic antagonist. Non-limiting examples of an alpha/beta blocker comprise labetalol (normodyne, trandate).

(3) Anti-Angiotension II Agents

Non-limiting examples of anti-angiotension II agents include include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.

(4) Sympatholytics

Non-limiting examples of a sympatholytic include a centrally acting sympatholytic or a peripherally acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).

(5) Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(β-diethylaminoethyl ether), hexobendine, itramin tosylate, khellin, LA-4195, lidoflanine, mannitol hexanitrane, medibazine, nicorglycerin, pentaerythritol tetranitrate, pentrinitrol, perhexyline, pimethylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

(6) Miscellaneous Antihypertensives

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a sulfonamide derivative.

Arylethanolamine Derivatives.

Non-limiting examples of arylethanolamine derivatives include amosulalol, bufuralol, dilevalol, labetalol, pronethalol, sotalol and sulfinalol.

Benzothiadiazine Derivatives.

Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-Carboxyalkyl(Peptide/Lactam) Derivatives.

Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives.

Non-limiting examples of dihydropyridine derivatives include amlodipine, felodipine, isradipine, nicardipine, nifedipine, nilvadipine, nisoldipine and nitrendipine.

Guanidine Derivatives.

Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines.

Non-limiting examples of hydrazines/phthalazines include budralazine, cadralazine, dihydralazine, endralazine, hydracarbazine, hydralazine, pheniprazine, pildralazine and todralazine.

Imidazole Derivatives.

Non-limiting examples of imidazole derivatives include clonidine, lofexidine, phentolamine, tiamenidine and tolonidine.

Quanternary Ammonium Compounds.

Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives.

Non-limiting examples of reserpine derivatives include bietaserpine, deserpidine, rescinnamine, reserpine and syrosingopine.

Sulfonamide Derivatives.

Non-limiting examples of sulfonamide derivatives include ambuside, clopamide, furosemide, indapamide, quinethazone, tripamide and xipamide.

(7) Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

g. Treatment Agents for Congestive Heart Failure

Non-limiting examples of agents for the treatment of congestive heart failure include anti-angiotension II agents, afterload-preload reduction treatment, diuretics and inotropic agents.

(1) Afterload-Preload Reduction

In certain embodiments, an animal patient that can not tolerate an angiotension antagonist may be treated with a combination therapy. Such therapy may combine administration of hydralazine (apresoline) and isosorbide dinitrate (isordil, sorbitrate).

(2) Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furtherene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexyline, ticmafen and urea.

(3) Inotropic Agents

Non-limiting examples of a positive inotropic agent, also known as a cardiotonic, include acefylline, an acetyldigitoxin, 2-amino-4-picoline, aminone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include enoximone and aminone (inocor).

(4) Antianginal Agents

Antianginal agents may comprise organonitrates, calcium channel blockers, beta blockers and combinations thereof.

Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

2. Surgical Therapeutic Agents

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

### IV. KITS

In some embodiments, there are kits for the detection of one or more NOS3 polymorphisms. In certain embodiments the polymorphism is at position 894. In some embodiments, the kits contain reagents specific for the detection or analysis of DNA (e.g., oligonucleotide probes or primers). In preferred embodiments, the kits contain all of the components necessary to perform a detection assay, including all controls, directions for performing assays, and any necessary software for analysis and presentation of results. In some embodiments, individual probes and reagents for detection of NOS3 polymorphisms are provided as analyte specific reagents. In other embodiments, the kits are provided as in vitro diagnostics.

### V. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### COMPARISON OF LA-419 AND ISDN

Donors and Cell Cultures

Human umbilical vein endothelial cells (HUVECs) were isolated into primary cultures and purchased as proliferating cells from Clonetics (San Diego, Calif.). All cell culture donors were healthy, with no pregnancy or prenatal complications. The cultured cells were incubated in 95% air/5% CO₂ at 37° C. and passage by an enzymatic (trypsin) procedure. The confluent cells (4 to 5×10⁵ cells per 35-mm dish) were placed with minimum essential medium containing 3 mM L-arginine and 0.1 mM BH₄ [(6R)-5,6,7,8-tetrahydrobiopterin]. Before the experiments, the cells (from second or third passage) were rinsed twice with Tyrode-HEPES buffer with 1.8 mM CaCl₂. ISDN was obtained from Sigma-Aldrich.

### Measurement of NO and ONOO⁻ Levels

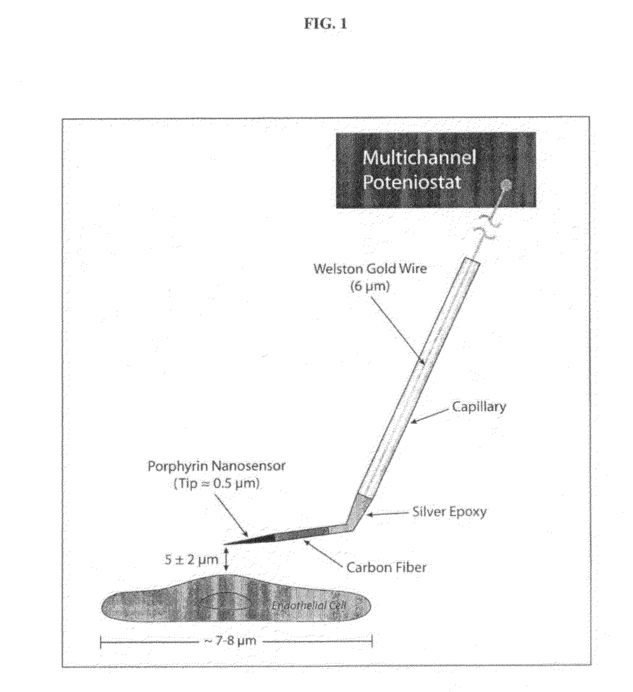

Concurrent measurements of NO and ONOO⁻ were carried out with tandem electrochemical nanosensors combined into one working unit with a total diameter of 200-500 nm (FIG. 1). Their design was based on previously developed and well-characterized chemically modified carbon-fiber technology (Malinski and Taha, 1992; Lvovich and Scheeline, 1997). Each of the nanosensors was made by depositing a sensing material on the tip of a carbon fiber (length 4-5 μm, diameter 0.2-0.5 μm). The fibers were sealed with nonconductive epoxy and electrically connected to copper wires with conductive silver epoxy. A conductive film of polymeric nickel (II) tetrakis(3-methoxy-4-hydroxyphenyl)porphyrin for the NO-sensor and polymeric film of Mn (III) [2.2]paracyclophanylporphyrin was used for the ONOO⁻-sensor.

A module of NO/ONOO⁻ nanosensors (diameter 1-2 μm) with a platinum wire (0.1 mm) counter electrode and saturated calomel reference electrode (SCE) were applied. Differential pulse voltammetry (DPV) and amperometry were performed with a computer-based Gamry VFP600 multichannel potentiostat. DPV was used to measure the basal NO and ONOO⁻ concentrations, and amperometry was used to measure changes in NO and ONOO⁻ concentrations from its basal level with time. The DPV current at the peak potential characteristic for NO oxidation (0.65 V) and ONOO⁻ reduction (−0.45 V) was directly proportional to the local concentrations of these compounds in the immediate vicinity of the sensor. Linear calibration curves (current vs. concentration) were constructed for each sensor from 10 nM to 3 μM before and after measurements with aliquots of NO and ONOO⁻ standard solutions, respectively. The detection limit of the sensors was 1.0 nM.

The quantification of each analyte (concentration in nM) was performed using a maximum current from amperograms and standard calibration curves. A reproducibility of measurements with nanosensors is relatively high (between 5-12%). The NO/ONOO⁻ nanosensor module was lowered with the help of a computer-controlled micromanipulator until it reached the surface of the cell membrane (a small piezoelectric signal, 0.1-0.2 pA, of 1-3 milliseconds duration was observed at this point). The sensors were slowly raised 5±2 μm from the surface of a single endothelial cell. The eNOS agonist, calcium ionophore A23187 (CaI) as well as the different drugs were then added to the surrounding media at the cell surface with a nanoinjector that was also positioned by a computer controlled-micromanipulator.

The HUVEC preparation is stable over the course of these experiments with the cells remaining viable in culture for >24 hours. Under non-stimulating conditions, basal levels of NO release are very low (<30 nM). Measurement of NO release as a function of treatment was conducted in individual endothelial cells. Multiple measurements of NO release can be conducted on single cells following a brief refractory period. For robust statistical analysis, separate cells were used for each concentration and type of drug used in these analyses.

### Results

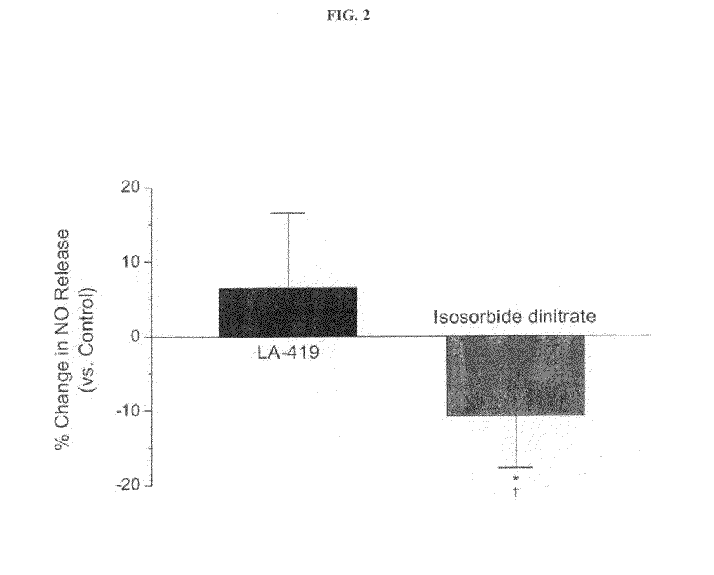

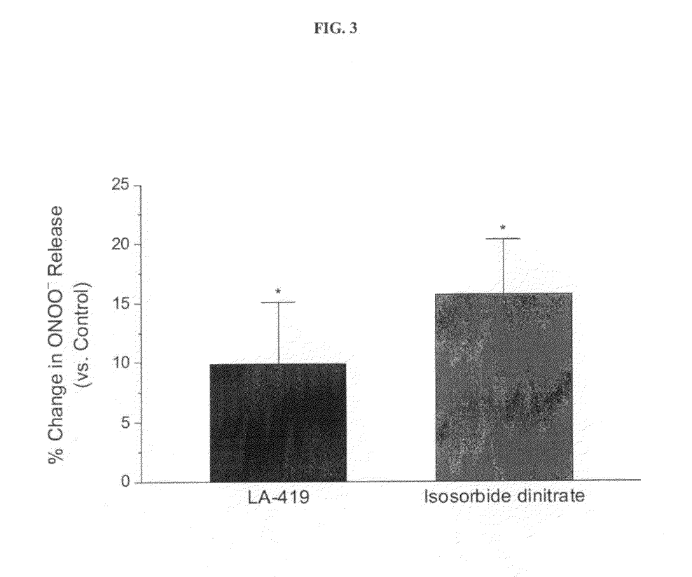

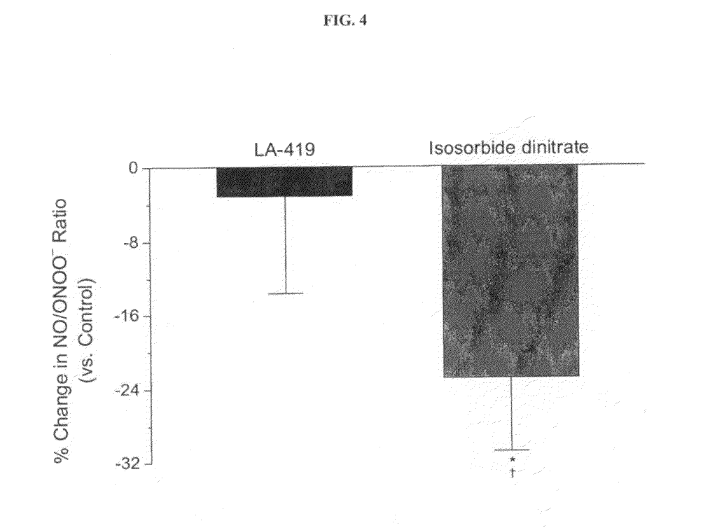

A comparison was made about the effects of ISDN versus LA-419 on endothelial-dependent NO and ONOO⁻ release in endothelium from white and African American donors. Exposure of human endothelial cells to ISDN for an extended period of time (12 h) had deleterious effects on their capacity to generate NO while causing a concomitant increase nitroxidative stress. These adverse effects of a conventional NO donor on endothelial function may contribute to mechanisms of tolerance and raise risk for atherothrombotic disease. Pretreatment of cells with ISDN resulted in a pronounced decrease in NO release by 11% (non-Hispanic whites) and 12% (African Americans) while increasing levels of ONOO⁻ by 16% (whites) and 18% (African Americans) (FIGS. 2-3). These effects resulted in an overall adverse effect of ISDN on eNOS function as evidenced by a decrease in the NO/ONOO⁻ ratio by 23% (non-Hispanic whites, p<0.01) and 25% (African Americans, p<0.05), as shown in FIG. 4. By contrast, treatment with LA-419 caused an opposite and small increase in NO release in HUVECs from both non-Hispanic whites and African Americans under identical conditions (FIGS. 2-3). As compared to ISDN, the NO/ONOO⁻ ratio was not significantly different from control samples (FIG. 4), thus not disrupting eNOS function. These findings indicate that ISDN has a detrimental effect on endothelial function and eNOS coupling mechanisms in a manner that was not observed with LA-419, even in cells from African American donors as shown in the Table below:

| | Non-Hispanic Whites | | African Americans | |
|---|---|---|---|---|
| Treatments | NO | ONOO⁻ | NO | ONOO⁻ |
| Control | 384 ± 24 | 279 ± 9 | 245 ± 13 | 468 ± 29 |
| LA-419 | 409 ± 30 | 307 ± 11 | 253 ± 15 | 532 ± 25 |
| ISDN | 343 ± 11 | 323 ± 9 | 218 ± 16 | 552 ± 11 |

Values are mean ± S.D. (units = nM)

These results of our analysis demonstrate an adverse effect of ISDN on endothelial function that may contribute to mechanisms of tolerance with use that was not observed with LA-419.

In these experiments LA-419 compared favorably against ISDN in its effect on endothelial-dependent NO bioavailability. In fact, these agents had opposite effects on eNOS function in cells from both white and African American donors who were well matched for CV risk. Thus, this is evidence that LA-419 produces less tolerance while maintaining vascular endothelial function compared to older nitrates such as ISDN.

### Example 2

### Effect of eNOS Variants with LA-419

This example describes the genotype-specific response of the three major eNOS variants to 1 μM and 5 μM concentrations of LA-419. The three major genetic polymorphisms studied were the Glu298Asp variant (E298D or 894G→T) in exon 7 of chromosome 17, the −786T→C variant in the promoter region, and the intron 4 polymorphism (VNTR). All of these variants have possible effects on the cardiovascular system and endothelial dysfunction. For this study, human umbilical vein endothelial cells (HUVECs) from 50 donors between 18 and 32 years of age were used.

For the analysis several analysis techniques were employed. First, the effect of genotype on NO and ONOO⁻ release was explored within the different concentrations of LA-419 (control, 1 μM, 5 μM), calculated as the average peak release (nmols/L) of NO, ONOO⁻, and NO/ONOO⁻. The effect of genotype on the net increase in NO, ONOO⁻, and NO/ONOO⁻ for LA-419 (1 μM and 5 μM) as compared to control was also measured. In addition, an analysis was done on the effect of genotype on the % change in NO, ONOO⁻, and NO/ONOO⁻ production for LA-419 (1 μM and 5 μM), calculated as:

% Change=[(avg. LA-419−avg. control)/(avg. control)]*100

Finally, an evaluation was undertaken concerning the effect of genotype on the production of NO, ONOO⁻, and NO/ONOO⁻ as the fold difference of control for LA-419 (1 μM and 5 μM), calculated as:

Fold Difference=(avg. LA-419)/(avg. control)

Within each calculation technique the results were compared for statistical significance using a non-parametric ANOVA Kruskal-Wallace test or a non-parametric Mann-Whitney U test. Statistical significance was set at a p-value <0.05 in a two-tailed distribution. Because experiment-experiment variability is reduced by the normalization to each experiment's control, the % change or fold difference measurements are considered more statistically robust than the net increase or decrease data.

### Materials and Methods

eNOS Activity and Protein Abundance Measurements

Same as Example 1.

eNOS Genotyping

The three major variants of the eNOS(NOS 3) gene that have possible effects on the cardiovascular system are Glu298Asp (E298D or 894G>T) in exon 7 of chromosome 17, −786T>C in the promoter region and intron 4 (VNTR). One million HUVECs supplied by Elucida and shipped frozen were used for DNA extraction. GE Healthcare's Illustra Tissue and Cells GenomicPrep Mini Spin Kit was used to isolate DNA for genotyping.

### Glu298Asp (G→T) Polymorphism

This exon 7 polymorphism is associated with acute myocardial infarction (AMI), increased risk for coronary heart disease (CHD), coronary spasm, early atherosclerosis, premature MI and abdominal aortic aneurism. Asp 298 (T894) is associated with AMI susceptibility, carotid atherosclerosis, CHD and recent MI's, severe CHD, early atherosclerosis, and coronary vasopastic angina (Napoli et al., 2006). All these conditions have in common endothelial dysfunction, and the Asp 298 allele is associated with endothelial dysfunction even in normal volunteers (Imamura et al., 2004) as well as in isolated human coronary arteries (Erbs et al., 2006). The mechanism by which the Asp 298 allele exerts these effects has not been determined.

Exon 7 genotypes were identified by PCR-RFLP methodology. The primers used were: Forward, aaggcaggagacagtggatgga (SEQ ID NO:5); Reverse, cccagtcaatccctttggtgctca (SEQ ID NO:6). The cycling conditions were 94° C. 4 minutes for 1 cycle; 94° C. 30 seconds, 65° C. 30 seconds, and 72° C. 1 minute for 35 cycles; and 72° C. for 5 minutes. PCR was followed by a restriction enzyme digest of the 258 bp product using Ban II to yield bands of 162 and 85 bp to indicate homozygous wild type, 258, 163, and 85 bp indicate a heterozygote and a homozygous mutant was uncut.

### −786 T→C Promoter Polymorphism

This promoter polymorphism is associated with an imbalance of autonomic activity in CHD (Napoli et al., 2006). T—C is associated with severe internal carotid artery disease but not with early atherosclerosis (Napoli et al., 2006). CC carriers have increased endothelial dysfunction due to less generation of NO (Erbs et al., 2006), which is likely to promote atherosclerosis and increase plaque formation.²

Promoter genotypes were also identified by PCR-RFLP methodology. The primers used were: Forward, tggagagtgctggtgtacccca (SEQ ID NO:7); Reverse, gcctccacccccaccctgtc (SEQ ID NO:8). The cycling conditions were 94° C. 4 minutes for 1 cycle; 94° C. 30 seconds, 65° C. 30 seconds, and 72° C. 1 minute for 35 cycles; and 72° C. for 5 minutes. PCR was followed by a restriction enzyme digest of the 180 bp product using MSP I to yield bands of 140 and 40 bp to indicate homozygous wild type, 140, 90, 50, and 40 bp indicate a heterozygote and a homozygous mutant gave bands of 90, 50, and 40 bp.

### Intron 4 Polymorphism

This variable tandem repeat polymorphism (“4a/4b”) may also be associated with acute coronary syndromes and CHD (Napoli et al., 2006) due to lower levels of NO production (Li et al., 2004).

Intron 4 variants were identified by PCR and gel electrophoresis. The primers used were: Forward, aggccctatggtagtgccttt (SEQ ID NO:9); Reverse, tctcttagtgctgtggtcac (SEQ ID NO:10). The cycling conditions were 94° C. 4 minutes for 1 cycle; 94° C. 30 seconds, 63° C. 30 seconds, and 72° C. 1 minute for 35 cycles; and 72° C. for 5 minutes. The wild type product, 4b/4b, yielded a 420 bp fragment. A heterozygote (4b/4a) gave bands of 420 and 393 bp. The mutant 4a/4a gave a 393 bp band; 4c/4c gave a 447 bp band and 4y/4y gave a 339 bp band.

### Results

Effect of Genotype within Group, Peak Release Measurements: (Group is Defined as Control, LA-419 5 μM, and LA-419 1 μM)

The effect of genotype on NO, ONOO⁻, and NO/ONOO⁻ generation by LA-419 as measured by peak release was assessed. As seen in tables a1-a3, in the control group (LA-419 vehicle alone), there were no significant differences in the levels of NO, ONOO⁻ and NO/ONOO⁻ between the gene variants of each polymorphism. Table b1 demonstrates an increase in the peak release of NO in response to LA-419 (5 μM), in all genotypes. A smaller increase in peak release of NO is produced in response to 1 μM LA-419 (Table c1). For average peak release, NO generation was not significantly different between genotypes in the three polymorphic sites, for either concentration of LA-419.

The levels of ONOO⁻ production revealed no apparent decrease in response to LA-419 (1 μM) whereas a decrease in ONOO⁻ production was detected in response to 5 μM LA-419 (Tables b2 and c2). There did not appear to be a genotype dose dependent decrease in peak release of ONOO⁻ for any of the polymorphisms.

The effects of genotype combinations on peak release of NO, ONOO⁻, and NO/ONOO⁻ within groups were also examined. For each polymorphism, major allele homozygotes were compared to minor allele carriers (Tables d-f). None of the measures were significantly different between major allele homozygotes and minor allele carriers, for either dose of LA-419.

Genotype effects on peak release of NO, ONOO⁻, and NO/ONOO⁻ were examined for the full allelic combinations of the exon 7 and promoter variants (Tables g-i). There was no apparent trend in a higher peak release of NO, decreased ONOO⁻ generation, or higher NO/ONOO⁻ in response to LA-419 (5 μM and 1 μM) for the exon 7 and promoter major allele combination (G/G+T/T) compared to other individual genotype combinations. Genotype effect on NO, ONOO⁻, and NO/ONOO⁻ levels was also examined for the major allele combination variant G/G+T/T compared to the combination of all other allelic variants, Tables j-l. There were no differences between the major allele combination variant and the other genotypes combined, for any peak release measure at either dose of LA-419.

### Effect of Genotype on Net Increase Over Control for 5 μM and 1 μM LA-419

The effect of genotype on the net increase in NO, ONOO⁻, and NO/ONOO⁻ levels for LA-419 (1 μM and 5 μM) over control is shown in Tables m-n. For net NO generation in response to 5 μM LA-419 (Table m1) there are nonsignificant trends for major allele-associated greater amounts of net NO release for both the exon 7 and promoter polymorphism. This possible genotype dependent response is not seen for 1 μM LA-419 (Table n1).

The effects of genotype combinations on the net increase in NO, ONOO⁻, and NO/ONOO⁻ levels for LA-419 (5 μM and 1 μM) over control were also examined (Tables o-p). The major allele gene variants for exon 7 and the promoter compared to their respective minor allele carriers showed a trend (p values of 0.10 and 0.12) for a greater (by 22% in each) net increase in NO in response to 5 μM LA-419 (Table o1). In addition, full allelic combinations of the exon 7 and promoter polymorphisms were examined (Tables q and r). In Table q1 it can be seen that the major allele combination variant, G/G+T/T, produced a nonsignificant, numerically greater net increase in NO in response to 5 μM LA-419 compared to the other individual allelic combinations. Furthermore, when the major allele variant G/G+T/T was compared to the combination of all other allelic variants the higher net increase in NO in response to 5 μM LA-419 remained, with a trend towards significance (p=0.13, Table s1). There were no significant differences in the net increase in ONOO⁻ for LA-419 (1 μM and 5 μM) over control for any of the genotype combinations.

### Effect of Genotype and Effect of Race on % Change for LA-419 (5 μM and 1 μM)

The effects of LA-419 on % change as compared to control are given in Tables u1-3 (5 μM LA-419) and v1-3 (1 μM LA-419). For 5 μM LA-419 the % increase in peak NO generation ranged from 11.6 to 17.4%, within genotype groups (Table u1). For the exon 7 G894T polymorphism, NO generation tended to be directly related to the presence of the G, major allele (p=0.12). Similarly, for the promoter T-786C variant there was also a trend (p=0.22) for NO generation to be related to the major allele. The intron 4 polymorphism pattern did not provide any evidence for an effect on NO generation. OONO⁻ generation did not appear to be related to any allele of any of the three polymorphic loci (Table u2). There was a nonsignificant trend for the biologically important NO/OONO⁻ ratio to be greater in the major allele homozygotes of both the exon 7 and promoter polymorphisms (Table u3), and in all genotypes the increase in ratio in response to 5 μM LA-419 was substantial, ranging from 37-53%. For 1 μM LA-419 there were no trends by genotype in degree of NO generation or NO/OONO⁻ enhancement (Table v1), which was more variable and tended to be less than for the 5 μM concentration.

Tables x1 and x2 give the derived measure of “% change ratio” for the eNOS product measures, calculated by dividing the % change in NO by the % change in OONO⁻. A positive value for this ratio means that either both the NO and OONO— values were positive or negative, and values <1.0 mean that the % decrease in OONO⁻ was >the increase in NO. For 5 μM LA-419 (Table x1) all values are negative, meaning that for all genotype groups in all polymorphisms NO generation was >control and OONO⁻ was <control. All ratios are <1.0, indicating a greater degree of OONO⁻ lowering than NO generation increase in all groups. Results for 1 μM LA-419 are more variable, reflecting the variability in both NO and OONO⁻ generation.

The effect of race on % change in NO, ONOO⁻, and NO/ONOO⁻ generation for the 5 μM LA-419 concentration (Tables u1-u3) demonstrated that Blacks tended to have lower levels of NO generation in comparison to other races. Among the self-identified races examined Blacks had the lowest % change in NO generation and the lowest % reduction in ONOO⁻, resulting in the lowest % change in NO/ONOO⁻ ratio (Tables u1-u3). Although in Blacks the % change in NO was not significantly different (p=0.66), the % change in ONOO⁻ and NO/ONOO⁻ were both significant at p=0.01. These trends within race were consistent in response to the 1 μM LA-419 concentration (Tables v1-v3), where Blacks were the only race that demonstrated an average decrease in NO generation and NO/OONO⁻ ratio (Tables v1 and v3),

### Effect of Genotype Combinations on Fold Difference for LA-419 (5 μM and 1 μM)

The complete analysis of genotypes and genotype combinations is presented as “Fold Difference” values, which gives the same result as % change with values (>1.0-1.0)×100 equaling % increase, and (1.0-<1.0)×100 values equaling % decrease. Tables y1-3 and z1-3 give identical results (after transformation) and p values as the results in Tables u1-3 and v1-3. The effects of genotype combinations on the fold difference of NO, ONOO⁻, and NO/ONOO⁻ generation for LA-419 are given in Tables aa-rr.

In Tables aa1-aa3 the major allele homozygotes were compared to the minor allele carriers for each polymorphism for response to 5 μM LA-419. Within the exon 7 comparison, the major allele homozygote had fold increase of NO production compared to minor allele carriers, 1.16±0.06 versus 1.13±0.06 (p=0.05). There was no significant difference in the percent of control for ONOO⁻ generation in response to 5 μM LA-419 for the major allele homozygote compared to the minor allele carriers within exon 7 (Table aa2). Accordingly, the major allele homozygote for exon 7 had a higher % of control for the NO/ONOO⁻ ratio in response to LA-419 (5 μM) compared to the minor allele carriers, 1.48±0.18 versus 1.39±0.13 (p=0.042, Table aa3). Trends of the same type were exhibited for the promoter variant comparison (Tables aa1-aa3), but with nonsignificant p values.

Genotype effects for the full allelic combinations of the exon 7 and promoter variants are given in Tables cc and dd. There was not a significant trend for NO or ONOO⁻ responses to LA-419 (either 5 μM and 1 μM) to be greater in the dominant allele combination (G/G+T/T) compared to all other genotypes. In fact, combining the major allele homozygotes weakened the statistical significance for the 5 μM LA-419 findings compared to the exon 7 dominant allele G/G genotype alone.

Genotype effect on fold difference was also examined for the major allele variant G/G+T/T compared to the combination of all other allelic variants, with the results given in Tables ee and f. The dominant allelic combination (G/G+T/T) tended to have a higher fold increase for NO production (p=0.09) and NO/ONOO⁻ ratio (p=0.13) in response to 5 μM LA-419 (Tables eel and ee3), but showed no trends for such effects in response to 1 μM LA-419 (Tables ff1 and ff3).

Effect of Genotype on LA-419 Fold Difference Responses within Caucasians

The effect of genotype on fold difference of NO, ONOO⁻, and NO/ONOO⁻ responses within Caucasians was examined, with data presented in Tables gg-nn. Table gg1 shows that both the exon 7 and promoter major allele variants have a numerically higher fold difference for NO production in response to 5 M LA-419, 1.19±0.07 (p=0.02) and 1.17±0.08 (p=0.15) respectively. Moreover, in Table gg3 it can be seen that that NO/ONOO⁻ ratios for the exon 7 and the promoter major alleles are numerically higher as compared to other genotypes, with respective p values of 0.06 and 0.07. There was no significant difference in the percent control for ONOO⁻ generation in response to LA-419 (5 μM) within Caucasians for any of the polymorphisms. These trends were not identified at the LA-419 1 μM concentration (Tables hh1-hh3).

Comparison of the effects of major allele homozygotes to minor allele carriers within Caucasians was also examined, with data presented in Tables ii and jj. Within the exon 7 comparison, the major allele homozygote had a fold difference for NO production in response to 5 μM LA-419 compared to the minor allele carriers of 1.19±0.07 versus 1.11±0.05, which was significantly different at a p=0.01 (Table ii1). There was no significant difference in the percent of control for ONOO⁻ generation in response to LA-419 (5 μM) for the major allele homozygote compared to the minor allele carriers within exon 7. Accordingly, the major allele homozygote for exon 7 had a fold difference for the NO/ONOO⁻ ratio in response to 5 μM LA-419 compared to the minor allele carriers, 1.47±0.12 versus 1.35±0.10 (p=0.027, Table ii3). Nonsignificant trends were present for the promoter variants, for both NO production (p=0.09, Table ii1)) and the NO/ONOO⁻ ratio (p=0.17, Table ll3). These trends were not seen at the LA-419 1 μM concentration, tables jj1-jj3.

Genotype effect on % of control within Caucasians was examined for the full allelic combinations of the exon 7 and promoter variants, as shown in Tables kk and ll. With the 5 μM LA-419 concentration there was a numerically higher fold difference for NO production (p=0.11, Table kk1) and the NO/ONOO⁻ ratio (p=0.0496, Table kk3) for the dominant allele combination, G/G+T/T. Similar trends were not present for 1 μM LA-419 (Tables ll1-ll3).

The major allele variant (G/G+T/T) data compared to the combination of all other allelic variants within Caucasians are given in Tables mm and nn. The major allelic combination exhibited trends for a higher fold difference in NO production (p=0.068, Table mm1) and NO/ONOO⁻ ratio (p=0.13, Table mm3) in response to 5 μM LA-419. The G/G+T/T combination genotype also had a trend for a higher fold difference for NO production in response to 1 μM LA-419 (p=0.076, Table nn1) but no trends for a higher NO/OONO⁻ ratio (Table nn3).

Effect of Genotype on Fold Difference in Minor Allele Homozygotes Vs. Major Allele Homozygotes and Heterozygotes

In all races the effect of genotype on fold difference of NO, ONOO—, and NO/ONOO— was examined within each polymorphism for the minor allele homozygotes compared to the combination of the major allele homozygotes and heterozygotes. For both the exon 7 and promoter polymorphism, the minor allele homozygote had a numerically lower but statistically nonsignificant fold difference for NO production and NO/ONOO⁻ ratio in response to 5 μM LA-419 (Tables oo1 and oo3). At the LA-419 1 μM concentration this trend was present for the promoter polymorphism, but was not apparent in the exon 7 polymorphism (Tables pp1 and pp3).

Genotype effects on fold difference for the minor allele homozygotes T/T+C/C compared to the combination of all other allelic variants (Tables qq and rr). There was no apparent trends in for differential fold differences for NO, ONOO⁻, and NO/ONOO⁻ in response to either dose of LA-419.

eNOS Protein Abundance

Using immunochemical methods, there was no change in any condition in relation to eNOS protein abundance, Tables ss-L1.

### Intron 4 Analysis

There was no gene allele dose related response to LA-419 (1 μM and 5 μM) for the intron 4 polymorphism, as presented in Tables M1-M4 and N1-N3 as well as tables throughout this report.

### APPENDIX

eNOS Activity Analysis
Effect of Genotype within Group

TABLES a1-a3
Effect of eNOS genotypes within control group*

| | | | | |
|---|---|---|---|---|
| | | Peak release, nmols/L | Chi-sq | |
| Variable | Level (n) | (mean ± sd) | statistic | p value |

a1)
NO (control)

| | | | | |
|---|---|---|---|---|
| G894T variant | G/G (26) | 343.87 (59.91) | 0.4138 | 0.81 |
| | G/T (18) | 354.76 (77.61) | | |
| | T/T (5) | 348.07 (100.76) | | |
| T-786C variant | T/T (22) | 349.92 (51.75) | 0.0975 | 0.95 |
| | T/C (23) | 349.55 (84.47) | | |
| | C/C (4) | 332.17 (80.88) | | |
| Intron 4 variant | b/b (31) | 353.53 (73.00) | 3.1640 | 0.367 |
| | b/a (10) | 320.67 (65.74) | | |
| | a/a (3) | 386.33 (72.90) | | |
| | c/c (1) | 368.67 (0) | | |

a2)
ONOO⁻ (control)

| | | | | |
|---|---|---|---|---|
| G894T variant | G/G (26) | 363.01 (84.61) | 3.3296 | 0.19 |
| | G/T (18) | 365.94 (87.41) | | |
| | T/T (5) | 449.27 (111.48) | | |
| T-786C variant | T/T (22) | 362.95 (86.39) | 0.6190 | 0.73 |
| | T/C (23) | 376.83 (91.28) | | |
| | C/C (4) | 404.92 (121.75) | | |
| Intron 4 variant | b/b (31) | 369.31 (86.00) | 0.9010 | 0.825 |
| | b/a (10) | 368.03 (100.38) | | |
| | a/a (3) | 395.00 (128.17) | | |
| | c/c (1) | 426.33 (0) | | |

a3)
NO/ONOO⁻ (control)

| | | | | |
|---|---|---|---|---|
| G894T variant | G/G (26) | 1.02 (0.34) | 0.6330 | 0.73 |
| | G/T (18) | 1.05 (0.37) | | |
| | T/T (5) | 0.85 (0.44) | | |
| T-786C variant | T/T (22) | 1.03 (0.33) | 0.6682 | 0.72 |
| | T/C (23) | 1.01 (0.39) | | |
| | C/C (4) | 0.92 (0.46) | | |
| Intron 4 variant | b/b (31) | 1.03 (0.36) | 0.2703 | 0.966 |
| | b/a (10) | 0.97 (0.41) | | |
| | a/a (3) | 1.07 (0.45) | | |
| | c/c (1) | 0.86 (0) | | |

Chi-sq statistic generated from the Non-parametric ANOVA Kruskal-Wallis test
*Control group is LA-419 vehicle.

TABLES b1-b3
Effect of eNOS genotypes within group LA-419 5 μM

| | | | | |
|---|---|---|---|---|
| | | Peak release, nmols/L | Chi-sq | |
| Variable | Level (n) | (mean ± sd) | statistic | p value |

b1)
NO (LA-419 5 μM)

| | | | | |
|---|---|---|---|---|
| G894T variant | G/G (26) | 398.92 (71.74) | 0.3530 | 0.84 |
| | G/T (18) | 400.63 (80.27) | | |
| | T/T (5) | 390.47 (124.39) | | |
| T-786C variant | T/T (22) | 405.83 (58.51) | 0.5336 | 0.77 |
| | T/C (23) | 396.81 (95.72) | | |
| | C/C (4) | 370.17 (86.78) | | |
| Intron 4 variant | b/b (31) | 400.83 (81.57) | 2.5597 | 0.465 |
| | b/a (10) | 375.20 (81.45) | | |
| | a/a (3) | 438.33 (79.68) | | |
| | c/c (1) | 432.67 (0) | | |

b2)
ONOO⁻ (LA-419 5 μM)

| | | | | |
|---|---|---|---|---|
| G894T variant | G/G (26) | 288.82 (78.22) | 2.5405 | 0.28 |
| | G/T (18) | 300.15 (75.86) | | |
| | T/T (5) | 366.40 (103.55) | | |
| T-786C variant | T/T (22) | 286.47 (76.65) | 1.5269 | 0.47 |
| | T/C (23) | 310.97 (82.59) | | |
| | C/C (4) | 322.33 (110.70) | | |
| Intron 4 variant | b/b (31) | 298.99 (79.66) | 1.4513 | 0.694 |
| | b/a (10) | 295.73 (85.11) | | |
| | a/a (3) | 331.22 (117.30) | | |
| | c/c (1) | 328.33 (0) | | |

b3)
NO/ONOO⁻ (LA-419 5 μM)

| | | | | |
|---|---|---|---|---|
| G894T variant | G/G (26) | 1.50 (0.51) | 1.3707 | 0.50 |
| | G/T (18) | 1.44 (0.49) | | |
| | T/T (5) | 1.21 (0.68) | | |
| T-786C variant | T/T (22) | 1.52 (0.45) | 0.8254 | 0.66 |
| | T/C (23) | 1.41 (0.56) | | |
| | C/C (4) | 1.32 (0.71) | | |
| Intron 4 variant | b/b (31) | 1.46 (0.51) | 0.3979 | 0.941 |
| | b/a (10) | 1.42 (0.61) | | |
| | a/a (3) | 1.48 (0.64) | | |
| | c/c (1) | 1.32 (0) | | |

Chi-sq statistic generated from the Non-parametric ANOVA Kruskal-Wallis test

TABLES c1-c3
Effect of eNOS genotypes within group LA-419 1 μM

| | | | | |
|---|---|---|---|---|
| | | Peak release, nmols/L | Chi-sq | |
| Variable | Level (n) | (mean ± sd) | statistic | p value |

c1)
NO (LA-419 1 μM)

| | | | | |
|---|---|---|---|---|
| G894T variant | G/G (26) | 367.13 (80.46) | 4.6995 | 0.095 |
| | G/T (18) | 413.28 (58.46) | | |
| | T/T (5) | 368.80 (75.73) | | |
| T-786C variant | T/T (22) | 380.73 (75.61) | 2.9185 | 0.23 |
| | T/C (23) | 396.32 (73.63) | | |
| | C/C (4) | 334.25 (68.06) | | |
| Intron 4 variant | b/b (31) | 393.18 (77.21) | 2.7011 | 0.440 |
| | b/a (10) | 377.23 (69.05) | | |
| | a/a (3) | 360.56 (80.60) | | |
| | c/c (1) | 270.00 (0) | | |

c2)
ONOO⁻ (LA-419 1 μM)

| | | | | |
|---|---|---|---|---|
| G894T variant | G/G (26) | 344.53 (83.88) | 1.1225 | 0.57 |
| | G/T (18) | 319.43 (81.89) | | |
| | T/T (5) | 332.67 (129.30) | | |
| T-786C variant | T/T (22) | 335.82 (80.98) | 3.5720 | 0.17 |
| | T/C (23) | 315.74 (78.13) | | |
| | C/C (4) | 430.17 (124.82) | | |
| Intron 4 variant | b/b (31) | 317.60 (74.77) | 3.6974 | 0.296 |
| | b/a (10) | 330.47 (88.93) | | |
| | a/a (3) | 421.11 (124.25) | | |
| | c/c (1) | 423.67 (0) | | |

c3)
NO/ONOO⁻ (LA-419 1 μM)

| | | | | |
|---|---|---|---|---|
| G894T variant | G/G (26) | 1.16 (0.45) | 1.8020 | 0.41 |
| | G/T (18) | 1.38 (0.38) | | |
| | T/T (5) | 1.29 (0.63) | | |
| T-786C variant | T/T (22) | 1.22 (0.43) | 3.3685 | 0.19 |
| | T/C (23) | 1.35 (0.44) | | |
| | C/C (4) | 0.88 (0.50) | | |
| Intron 4 variant | b/b (31) | 1.33 (0.42) | 2.8821 | 0.410 |
| | b/a (10) | 1.25 (0.46) | | |
| | a/a (3) | 0.97 (0.57) | | |
| | c/c (1) | 0.64 (0) | | |

Chi-sq statistic generated from the Non-parametric ANOVA Kruskal-Wallis test

TABLES d1-d3
Effect of genotype within group control. Comparison between
major allele homozygotes and minor allele carriers

| | | | | |
|---|---|---|---|---|
| | | Peak release, | | |
| | | nmols/L | | |
| Variable | Level (n) | (mean ± sd) | t statistic | p value |

d1)
NO (control)

| | | | | |
|---|---|---|---|---|
| G894T variant | G/G (26) | 343.87 (59.91) | 0.5710 | 0.57 |
| | G/T & T/T (23) | 353.30 (80.67) | | |
| T-786C variant | T/T (22) | 349.92 (51.75) | 0.0402 | 0.97 |
| | T/C & C/C (27) | 346.98 (82.66) | | |
| Intron 4 variant | b/b (31) | 353.53 (73.00) | −0.7110 | 0.481 |
| | b/a, a/a, c/c (14) | 338.17 (68.21) | | |

d2)
ONOO⁻ (control)

| | | | | |
|---|---|---|---|---|
| G894T variant | G/G (26) | 363.01 (84.61) | 0.8014 | 0.43 |
| | G/T & T/T (23) | 384.06 (96.95) | | |
| T-786C variant | T/T (22) | 362.95 (86.39) | −0.7035 | 0.49 |
| | T/C & C/C (27) | 380.99 (94.15) | | |
| Intron 4 variant | b/b (31) | 369.31 (86.00) | 0.1103 | 0.913 |
| | b/a, a/a, c/c (14) | 377.98 (99.12) | | |

d3)
NO/ONOO⁻ (control)

| | | | | |
|---|---|---|---|---|
| G894T variant | G/G (26) | 1.02 (0.34) | −0.2104 | 0.83 |
| | G/T & T/T (23) | 1.01 (0.39) | | |
| T-786C variant | T/T (22) | 1.03 (0.33) | 0.3518 | 0.73 |
| | T/C & C/C (27) | 1.00 (0.39) | | |
| Intron 4 variant | b/b (31) | 1.03 (0.36) | −0.0123 | 0.990 |
| | b/a, a/a, c/c (14) | 0.99 (0.39) | | |

t statistic generated from the Non-parametric Mann-Whitney U test

TABLES e1-e3
Effect of genotype within group LA-419 (5 μM). Comparison between
major allele homozygotes and minor allele carriers

| | | | | |
|---|---|---|---|---|
| | | Peak release, | | |
| | | nmols/L | | |
| Variable | Level (n) | (mean ± sd) | t statistic | p value |

e1)
NO (LA-419 5 μM)

| | | | | |
|---|---|---|---|---|
| G894T variant | G/G (26) | 398.92 (71.74) | −0.0100 | 0.99 |
| | G/T & T/T (23) | 398.42 (88.37) | | |
| T-786C variant | T/T (22) | 405.83 (58.51) | 0.1709 | 0.85 |
| | T/C & C/C (27) | 392.86 (93.35) | | |
| Intron 4 variant | b/b (31) | 400.83 (81.57) | −0.1103 | 0.913 |
| | b/a, a/a, c/c (14) | 392.83 (80.05) | | |

e2)
ONOO⁻ (LA-419 5 μM)

| | | | | |
|---|---|---|---|---|
| G894T variant | G/G (26) | 288.82 (78.22) | 1.1219 | 0.27 |
| | G/T & T/T (23) | 314.55 (84.72) | | |
| T-786C variant | T/T (22) | 286.47 (76.65) | −1.2161 | 0.23 |
| | T/C & C/C (27) | 312.65 (84.87) | | |
| Intron 4 variant | b/b (31) | 298.99 (79.66) | 0.2329 | 0.817 |
| | b/a, a/a, c/c (14) | 305.67 (86.01) | | |

e3)
NO/ONOO⁻ (LA-419 5 μM)

| | | | | |
|---|---|---|---|---|
| G894T variant | G/G (26) | 1.50 (0.51) | −0.9917 | 0.33 |
| | G/T & T/T (23) | 1.39 (0.53) | | |
| T-786C variant | T/T (22) | 1.52 (0.45) | 0.8744 | 0.39 |
| | T/C & C/C (27) | 1.39 (0.57) | | |
| Intron 4 variant | b/b (31) | 1.46 (0.51) | 0.1103 | 0.913 |
| | b/a, a/a, c/c (14) | 1.43 (0.57) | | |

t statistic generated from the Non-parametric Mann-Whitney U test

TABLES f1-f3
Effect of genotype within group LA-419 (1 μM). Comparison between
major allele homozygotes and minor allele carriers

| | | | | |
|---|---|---|---|---|
| | | Peak release, | | |
| | | nmols/L | | |
| Variable | Level (n) | (mean ± sd) | t statistic | p value |

f1)
NO (LA-419 1 μM)

| | | | | |
|---|---|---|---|---|
| G894T variant | G/G (26) | 367.13 (80.46) | 1.7730 | 0.08 |
| | G/T & T/T (23) | 403.61 (63.52) | | |
| T-786C variant | T/T (22) | 380.73 (75.61) | −0.6332 | 0.53 |
| | T/C & C/C (27) | 387.12 (75.01) | | |
| Intron 4 variant | b/b (31) | 393.18 (77.21) | −1.0420 | 0.303 |
| | b/a, a/a, c/c (14) | 366.00 (71.50) | | |

f2)
ONOO⁻ (LA-419 1 μM)

| | | | | |
|---|---|---|---|---|
| G894T variant | G/G (26) | 344.53 (83.88) | −1.0118 | 0.32 |
| | G/T & T/T (23) | 322.30 (90.85) | | |
| T-786C variant | T/T (22) | 335.82 (80.98) | 0.3719 | 0.71 |
| | T/C & C/C (27) | 332.69 (93.16) | | |
| Intron 4 variant | b/b (31) | 317.60 (74.77) | 1.1402 | 0.260 |
| | b/a, a/a, c/c (14) | 356.55 (98.40) | | |

f3)
NO/ONOO⁻ (LA-419 1 μM)

| | | | | |
|---|---|---|---|---|
| G894T variant | G/G (26) | 1.16 (0.45) | 1.3322 | 0.19 |
| | G/T & T/T (23) | 1.36 (0.43) | | |
| T-786C variant | T/T (22) | 1.22 (0.43) | −0.4121 | 0.68 |
| | T/C & C/C (27) | 1.28 (0.47) | | |
| Intron 4 variant | b/b (31) | 1.33 (0.42) | −0.9439 | 0.350 |
| | b/a, a/a, c/c (14) | 1.15 (0.48) | | |

t statistic generated from the Non-parametric Mann-Whitney U test

TABLES g1-g3
Effect of genotype combinations within group control (Individual
allelic combination). Reference group = G/G + T/T

| | | | | |
|---|---|---|---|---|
| | | Peak release, | | |
| | | nmols/L | Chi-sq | |
| Variable | Level (n) | (mean ± sd) | statistic | p value |

g1)
NO (control)

| | | | | |
|---|---|---|---|---|
| G894T + | G/G + T/T (18) | 343.07 (49.58) | 3.0055 | 0.81 |
| T-786C | G/G + T/C (8) | 345.67 (82.72) | | |
| variant | G/G/+C/C (0) | — | | |
| | G/T + T/T (4) | 380.75 (57.17) | | |
| | G/T + T/C (13) | 344.18 (85.29) | | |
| | G/T + C/C (1) | 388.33 (0) | | |
| | T/T + T/C (2) | 400.00 (127.28) | | |
| | T/T + C/C (3) | 313.44 (87.80) | | |

g2)
ONOO⁻ (control)

| | | | | |
|---|---|---|---|---|
| G894T + | G/G + T/T (18) | 371.52 (91.89) | 5.5092 | 0.48 |
| T-786C | G/G + T/C (8) | 343.88 (66.73) | | |
| variant | G/G/+C/C (0) | — | | |
| | G/T + T/T (4) | 324.42 (44.40) | | |
| | G/T + T/C (13) | 383.92 (95.01) | | |
| | G/T + C/C (1) | 298.33 (0) | | |
| | T/T + T/C (2) | 462.50 (140.71) | | |
| | T/T + C/C (3) | 440.44 (121.09) | | |

g3)
NO/ONOO⁻ (control)

| | | | | |
|---|---|---|---|---|
| G894T + | G/G + T/T (18) | 1.00 (0.34) | 2.4033 | 0.88 |
| T-786C | G/G + T/C (8) | 1.07 (0.37) | | |
| variant | G/G/+C/C (0) | — | | |
| | G/T + T/T (4) | 1.19 (0.25) | | |
| | G/T + T/C (13) | 0.98 (0.40) | | |
| | G/T + C/C (1) | 1.3016760 (0) | | |
| | T/T + T/C (2) | 0.95 (0.56) | | |
| | T/T + C/C (3) | 0.79 (0.46) | | |

Chi-sq statistic generated from the Non-parametric ANOVA Kruskal-Wallis test

TABLES h1-h3
Effect of genotype combinations within group LA-419 5 μM (Individual
allelic combination). Reference group = G/G + T/T

| | | | | |
|---|---|---|---|---|
| | | Peak release, | | |
| | | nmols/L | Chi-sq | |
| Variable | Level (n) | (mean ± sd) | statistic | p value |

h1)
NO (LA-419 5 μM)

| | | | | |
|---|---|---|---|---|
| G894T + T-786C | G/G + T/T (18) | 399.67 (56.96) | 2.7891 | 0.83 |
| variant | G/G + T/C (8) | 397.25 (102.46) | | |
| | G/G/+C/C (0) | — | | |
| | G/T + T/T (4) | 433.58 (65.75) | | |
| | G/T + T/C (13) | 387.90 (86.10) | | |
| | G/T + C/C (1) | 434.33 (0) | | |
| | T/T + T/C (2) | 453.00 (178.19) | | |
| | T/T + C/C (3) | 348.78 (92.48) | | |

h2)
ONOO⁻ (LA-419 5 μM)

| | | | | |
|---|---|---|---|---|
| G894T + T-786C | G/G + T/T (18) | 293.93 (83.07) | 5.1552 | 0.5241 |
| variant | G/G + T/C (8) | 277.33 (69.85) | | |
| | G/G/+C/C (0) | — | | |
| | G/T + T/T (4) | 252.92 (13.69) | | |
| | G/T + T/C (13) | 318.33 (82.55) | | |
| | G/T + C/C (1) | 252.67 (0) | | |
| | T/T + T/C (2) | 397.67 (96.64) | | |
| | T/T + C/C (3) | 345.56 (123.07) | | |

h3)
NO/ONOO⁻ (LA-419 5 μM)

| | | | | |
|---|---|---|---|---|
| G894T + T-786C | G/G + T/T (18) | 1.48 (0.48) | 3.1462 | 0.79 |
| variant | G/G + T/C (8) | 1.56 (0.60) | | |
| | G/G/+C/C (0) | — | | |
| | G/T + T/T (4) | 1.71 (0.21) | | |
| | G/T + T/C (13) | 1.34 (0.54) | | |
| | G/T + C/C (1) | 1.72 (0) | | |
| | T/T + T/C (2) | 1.23 (0.75) | | |
| | T/T + C/C (3) | 1.19 (0.80) | | |

Chi-sq statistic generated from the Non-parametric ANOVA Kruskal-Wallis test

TABLES i1-i3
Effect of genotype combinations within group LA-419 1 μM (Individual
allelic combination). Reference group = G/G + T/T

| | | | | |
|---|---|---|---|---|
| | | Peak release, | | |
| | | nmols/L | Chi-sq | |
| Variable | Level (n) | (mean ± sd) | statistic | p value |

i1)
NO (LA-419 1 μM)

| | | | | |
|---|---|---|---|---|
| G894T + T-786C | G/G + T/T (18) | 374.30 (78.06) | 7.3555 | 0.29 |
| variant | G/G + T/C (8) | 351.00 (88.83) | | |
| | G/G/+C/C (0) | — | | |
| | G/T + T/T (4) | 409.67 (64.25) | | |
| | G/T + T/C (13) | 420.44 (56.87) | | |
| | G/T + C/C (1) | 334.67 (0) | | |
| | T/T + T/C (2) | 420.83 (4.48) | | |
| | T/T + C/C (3) | 334.11 (83.36) | | |

i2)
ONOO⁻ (LA-419 1 μM)

| | | | | |
|---|---|---|---|---|
| G894T + T-786C | G/G + T/T (18) | 338.98 (81.16) | 9.3929 | 0.15 |
| variant | G/G + T/C (8) | 357.00 (94.18) | | |
| | G/G/+C/C (0) | — | | |
| | G/T + T/T (4) | 321.58 (90.82) | | |
| | G/T + T/C (13) | 302.82 (59.66) | | |
| | G/T + C/C (1) | 526.67 (0) | | |
| | T/T + T/C (2) | 234.67 (23.10) | | |
| | T/T + C/C (3) | 398.00 (131.00) | | |

i3)
NO/ONOO⁻ (LA-419 1 μM)

| | | | | |
|---|---|---|---|---|
| G894T + T-786C | G/G + T/T (18) | 1.19 (0.43) | 9.1043 | 0.17 |
| variant | G/G + T/C (8) | 1.09 (0.51) | | |
| | G/G/+C/C (0) | — | | |
| | G/T + T/T (4) | 1.37 (0.44) | | |
| | G/T + T/C (13) | 1.44 (0.32) | | |
| | G/T + C/C (1) | 0.64 (0) | | |
| | T/T + T/C (2) | 1.80 (0.16) | | |
| | T/T + C/C (3) | 0.96 (0.58) | | |

Chi-sq statistic generated from the Non-parametric ANOVA Kruskal-Wallis test

TABLES j1-j3
Effect of genotype combinations within group control (reference vs. all
carriers combination).
Reference group = G/G + T/T.
Combination = G/G + T/C, G/G/+C/C, G/T + T/T, G/T + T/C,
G/T + C/C, T/T + T/C, T/T + C/C

| | | | | |
|---|---|---|---|---|
| | | Peak release, nmols/L | t | |
| Variable | Level (n) | (mean ± sd) | statistic | p value |

j1)
NO (control)

| | | | | |
|---|---|---|---|---|
| Genotype | Reference (18) | 343.07 (49.58) | −0.5911 | 0.556 |
| | Combination (31) | 351.33 (79.88) | | |

j2)
ONOO⁻ (control)

| | | | | |
|---|---|---|---|---|
| Genotype | Reference (18) | 371.52 (91.89) | −0.2489 | 0.80 |
| | Combination (31) | 373.69 (90.83) | | |

j3)
NO/ONOO⁻ (control)

| | | | | |
|---|---|---|---|---|
| Genotype | Reference (18) | 1.00 (0.34) | −0.0933 | 0.93 |
| | Combination (31) | 1.02 (0.38) | | |

t statistic generated from the Non-parametric Mann-Whitney U test

TABLES k1-k3
Effect of genotype combinations within group LA-419 5 μM
(reference vs. all carriers combination).
Reference group = G/G + T/T.
Combination = G/G + T/C, G/G/+C/C, G/T + T/T, G/T + T/C,
G/T + C/C, T/T + T/C, T/T + C/C

| | | | | |
|---|---|---|---|---|
| | | Peak release, nmols/L | t | |
| Variable | Level (n) | (mean ± sd) | statistic | p value |

k1)
NO (LA-419 5 μM)

| | | | | |
|---|---|---|---|---|
| Genotype | Reference (18) | 399.67 (56.96) | −0.3629 | 0.72 |
| | Combination (31) | 398.12 (90.43) | | |

k2)
ONOO⁻ (LA-419 5 μM)

| | | | | |
|---|---|---|---|---|
| Genotype | Reference (18) | 293.93 (83.07) | −0.7259 | 0.47 |
| | Combination (31) | 304.95 (81.70) | | |

k3)
NO/ONOO⁻ (LA-419 5 μM)

| | | | | |
|---|---|---|---|---|
| Genotype | Reference (18) | 1.48 (0.48) | 0.3837 | 0.70 |
| | Combination (31) | 1.43 (0.54) | | |

t statistic generated from the Non-parametric Mann-Whitney U test

TABLES l1-l3
Effect of genotype combinations within group LA-419 1 μM
(reference vs. all carriers combination).
Reference group = G/G + T/T.
Combination = G/G + T/C, G/G/+C/C, G/T + T/T, G/T + T/C,
G/T + C/C, T/T + T/C, T/T + C/C

| | | | | |
|---|---|---|---|---|
| | | Peak release, nmols/L | t | |
| Variable | Level (n) | (mean ± sd) | statistic | p value |

l1)
NO (LA-419 1 μM)

| | | | | |
|---|---|---|---|---|
| Genotype | Reference (18) | 374.30 (78.06) | −1.1096 | 0.27 |
| | Combination (31) | 390.03 (73.13) | | |

l2)
ONOO⁻ (LA-419 1 μM)

| | | | | |
|---|---|---|---|---|
| Genotype | Reference (18) | 338.98 (81.16) | 0.4667 | 0.64 |
| | Combination (31) | 331.26 (91.43) | | |

l3)
NO/ONOO⁻ (LA-419 1 μM)

| | | | | |
|---|---|---|---|---|
| Genotype | Reference (18) | 1.19 (0.43) | −0.7155 | 0.48 |
| | Combination (31) | 1.29 (0.46) | | |

t statistic generated from the Non-parametric Mann-Whitney U test

### Net Increase Over Control

TABLES m1-m3
Net increase in 5 μM LA-419 over control - comparison between
genotypes

| | | | | |
|---|---|---|---|---|
| | | Net increase, nmols/L | Chi-sq | |
| Variable | Level (n) | (mean ± sd) | statistic | p value |

m1)
NO (5 μM LA-419)

| | | | | |
|---|---|---|---|---|
| G894T variant | G/G (26) | 55.05 (21.03) | 2.9488 | 0.23 |
| | G/T (18) | 45.87 (19.24) | | |
| | T/T (5) | 42.40 (30.77) | | |
| T-786C variant | T/T (22) | 55.91 (19.06) | 3.1740 | 0.20 |
| | T/C (23) | 47.26 (23.70) | | |
| | C/C (4) | 38.00 (17.39) | | |
| Intron 4 variant | b/b (31) | 47.30 (22.42) | 1.2423 | 0.743 |
| | b/a (10) | 54.53 (23.50) | | |
| | a/a (3) | 52.00 (16.74) | | |
| | c/c (1) | 64.00 (0) | | |

m2)
ONOO⁻ (5 μM LA-419)

| | | | | |
|---|---|---|---|---|
| G894T variant | G/G (26) | −74.19 (29.80) | 1.7603 | 0.41 |
| | G/T (18) | −65.80 (26.77) | | |
| | T/T (5) | −82.87 (28.56) | | |
| T-786C variant | T/T (22) | −76.48 (33.23) | 1.7456 | 0.42 |
| | T/C (23) | −65.86 (23.41) | | |
| | C/C (4) | −82.58 (26.16) | | |
| Intron 4 variant | b/b (31) | −70.32 (30.56) | 1.4292 | 0.699 |
| | b/a (10) | −72.30 (25.81) | | |
| | a/a (3) | −63.78 (14.11) | | |
| | c/c (1) | −98.00 (0) | | |

m3)
NO/ONOO⁻ (5 μM LA-419)

| | | | | |
|---|---|---|---|---|
| G894T variant | G/G (26) | −0.85 (0.52) | 2.3998 | 0.30 |
| | G/T (18) | −0.94 (0.88) | | |
| | T/T (5) | −0.78 (1.05) | | |
| T-786C variant | T/T (22) | −0.95 (0.67) | 2.0061 | 0.37 |
| | T/C (23) | −0.86 (0.80) | | |
| | C/C (4) | −0.52 (0.36) | | |
| Intron 4 variant | b/b (31) | 0.43 (0.20) | 0.0468 | 0.997 |
| | b/a (10) | 0.45 (0.22) | | |
| | a/a (3) | 0.41 (0.19) | | |
| | c/c (1) | 0.46 (0) | | |

Net increase = avg 5 μM − avg Cntl. Chi-sq statistic generated from the Non-parametric ANOVA Kruskal-Wallis test

TABLES n1-n3
Net increase in 1 μM LA-419 over control - comparison between
genotypes

| | | | | |
|---|---|---|---|---|
| | | Net increase, nmols/L | Chi-sq | |
| Variable | Level (n) | (mean ± sd) | statistic | p value |

n1)
NO (1 μM LA-419)

| | | | | |
|---|---|---|---|---|
| G894T variant | G/G (26) | 23.26 (70.68) | 1.1059 | 0.58 |
| | G/T (18) | 58.52 (72.43) | | |
| | T/T (5) | 20.73 (66.94) | | |
| T-786C variant | T/T (22) | 30.80 (64.85) | 2.6735 | 0.26 |
| | T/C (23) | 46.77 (81.32) | | |
| | C/C (4) | 2.08 (39.61) | | |
| Intron 4 variant | b/b (31) | 39.66 (70.27) | 3.6920 | 0.297 |
| | b/a (10) | 56.57 (67.97) | | |
| | a/a (3) | −25.78 (104.49) | | |
| | c/c (1) | −98.67 (0) | | |

n2)
ONOO⁻ (1 μM LA-419)

| | | | | |
|---|---|---|---|---|
| G894T variant | G/G (26) | −18.49 (93.20) | 3.2587 | 0.20 |
| | G/T (18) | −46.52 (105.03) | | |
| | T/T (5) | −116.60 (117.55) | | |
| T-786C variant | T/T (22) | −27.14 (82.12) | 2.5212 | 0.28 |
| | T/C (23) | −61.09 (111.68) | | |
| | C/C (4) | 25.25 (135.63) | | |
| Intron 4 variant | b/b (31) | −51.71 (108.03) | 2.0897 | 0.554 |
| | b/a (10) | −37.57 (99.15) | | |
| | a/a (3) | 26.11 (116.99) | | |
| | c/c (1) | −2.67 (0) | | |

n3)
NO/ONOO⁻ (1 μM LA-419)

| | | | | |
|---|---|---|---|---|
| G894T variant | G/G (26) | 0.53 (7.59) | 3.9613 | 0.14 |
| | G/T (18) | −1.12 (1.02) | | |
| | T/T (5) | −0.31 (0.61) | | |
| T-786C variant | T/T (22) | 0.77 (8.26) | 2.2432 | 0.33 |
| | T/C (23) | −0.99 (0.94) | | |
| | C/C (4) | −0.48 (0.48) | | |
| Intron 4 variant | b/b (31) | 0.29 (0.47) | 3.0652 | 0.382 |
| | b/a (10) | 0.28 (0.43) | | |
| | a/a (3) | −0.11 (0.56) | | |
| | c/c (1) | −0.23 (0) | | |

Net increase = avg 1 μM − avg Cntl. Chi-sq statistic generated from the Non-parametric ANOVA Kruskal-Wallis test

TABLES o1-o3
Net increase in 5 μM LA-419 over control - comparison between major
allele homozygotes and minor allele carriers

| | | | | |
|---|---|---|---|---|
| | | Net increase, | | |
| | | nmols/L | | |
| Variable | Level (n) | (mean ± sd) | t statistic | p value |

o1)
NO (5 μM LA-419)

| | | | | |
|---|---|---|---|---|
| G894T variant | G/G (26) | 55.05 (21.03) | −1.6829 | 0.10 |
| | G/T & T/T (23) | 45.12 (21.46) | | |
| T-786C variant | T/T (22) | 55.91 (19.06) | 1.5780 | 0.12 |
| | T/C & C/C (27) | 45.89 (22.83) | | |
| Intron 4 variant | b/b (31) | 47.30 (22.42) | 0.7723 | 0.444 |
| | b/a, a/a, c/c (14) | 47.30 (22.42) | | |

o2)
ONOO⁻ (5 μM LA-419)

| | | | | |
|---|---|---|---|---|
| G894T variant | G/G (26) | −74.19 (29.80) | 0.4107 | 0.68 |
| | G/T & T/T (23) | −69.51 (27.46) | | |
| T-786C variant | T/T (22) | −76.48 (33.23) | −0.7839 | 0.44 |
| | T/C & C/C (27) | −68.33 (24.07) | | |
| Intron 4 variant | b/b (31) | −70.32 (30.56) | −0.4904 | 0.626 |
| | b/a, a/a, c/c (14) | −72.31 (23.65) | | |

o3)
NO/ONOO⁻ (5 μM LA-419)

| | | | | |
|---|---|---|---|---|
| G894T variant | G/G (26) | −0.85 (0.52) | 1.0117 | 0.32 |
| | G/T & T/T (23) | −0.91 (0.90) | | |
| T-786C variant | T/T (22) | −0.95 (0.67) | −1.0754 | 0.29 |
| | T/C & C/C (27) | −0.81 (0.76) | | |
| Intron 4 variant | b/b (31) | 0.43 (0.20) | 0.1594 | 0.874 |
| | b/a, a/a, c/c (14) | 0.44 (0.20) | | |

Net increase = avg 5 μM − avg Cntl. t statistic generated from the Non-parametric Mann-Whitney U test

TABLES p1-p3
Net increase in 1 μM LA-419 over control - comparison between major
allele homozygotes and minor allele carriers

| | | | | |
|---|---|---|---|---|
| | | Net increase, | | |
| | | nmols/L | t | |
| Variable | Level (n) | (mean ± sd) | statistic | p value |

p1)
NO (1 μM LA-419)

| | | | | |
|---|---|---|---|---|
| G894T variant | G/G (26) | 23.26 (70.64) | 0.2204 | 0.83 |
| | G/T & T/T (23) | 50.30 (71.57) | | |
| T-786C variant | T/T (22) | 30.80 (64.85) | 0.1307 | 0.90 |
| | T/C & C/C (27) | 40.15 (77.70) | | |
| Intron 4 variant | b/b (31) | 39.66 (70.27) | −0.1103 | 0.913 |
| | b/a, a/a, c/c (14) | 27.83 (86.07) | | |

p2)
ONOO⁻ (1 μM LA-419)

| | | | | |
|---|---|---|---|---|
| G894T variant | G/G (26) | −18.49 (93.20) | −1.2321 | 0.22 |
| | G/T & T/T (23) | −61.75 (109.14) | | |
| T-786C variant | T/T (22) | −27.14 (82.12) | 1.2664 | 0.21 |
| | T/C & C/C (27) | −48.30 (116.85) | | |
| Intron 4 variant | b/b (31) | −51.71 (108.03) | 0.6620 | 0.511 |
| | b/a, a/a, c/c (14) | −21.43 (98.29) | | |

p3)
NO/ONOO⁻ (1 μM LA-419)

| | | | | |
|---|---|---|---|---|
| G894T variant | G/G (26) | 0.53 (7.59) | 0.3105 | 0.76 |
| | G/T & T/T (23) | −0.93 (0.99) | | |
| T-786C variant | T/T (22) | 0.77 (8.26) | −0.3518 | 0.73 |
| | T/C & C/C (27) | −0.91 (0.90) | | |
| Intron 4 variant | b/b (31) | 0.29 (0.47) | −0.6252 | 0.535 |
| | b/a, a/a, c/c (14) | 0.16 (0.46) | | |

Net increase = avg 1 μM − avg Cntl. t statistic generated from the Non-parametric Mann-Whitney U test

TABLES q1-q3
Net increase in 5 μM LA-419 over control (Individual allelic
combination).
Reference group = G/G + T/T

| | | | | |
|---|---|---|---|---|
| | | Net increase, | | |
| | | nmols/L | Chi-sq | |
| Variable | Level (n) | (mean ± sd) | statistic | p value |

q1)
NO (5 μM LA-419 increase over Control)

| | | | | |
|---|---|---|---|---|
| G894T + T-786C | G/G + T/T (18) | 56.59 (19.21) | 4.2869 | 0.64 |
| variant | G/G + T/C (8) | 51.58 (25.77) | | |
| | G/G/+C/C (0) | — | | |
| | G/T + T/T (4) | 52.83 (20.89) | | |
| | G/T + T/C (13) | 43.72 (19.86) | | |
| | G/T + C/C (1) | 46.00 (0) | | |
| | T/T + T/C (2) | 53.00 (50.91) | | |
| | T/T + C/C (3) | 35.33 (20.27) | | |

q2)
ONOO⁻ (5 μM LA-419 increase over Control)

| | | | | |
|---|---|---|---|---|
| G894T + T-786C | G/G + T/T (18) | −77.59 (31.16) | 5.1514 | 0.52 |
| variant | G/G + T/C (8) | −66.54 (26.77) | | |
| | G/G/+C/C (0) | — | | |
| | G/T + T/T (4) | −71.50 (46.74) | | |
| | G/T + T/C (13) | −65.59 (20.60) | | |
| | G/T + C/C (1) | −45.67 (0) | | |
| | T/T + T/C (2) | −64.83 (44.08) | | |
| | T/T + C/C (3) | −94.89 (10.87) | | |

q3)
NO/ONOO⁻ (5 μM LA-419 increase over Control)

| | | | | |
|---|---|---|---|---|
| G894T + T-786C | G/G + T/T (18) | −0.88 (0.54) | 4.8102 | 0.57 |
| variant | G/G + T/C (8) | −0.78 (0.51) | | |
| | G/G/+C/C (0) | — | | |
| | G/T + T/T (4) | −1.29 (1.15) | | |
| | G/T + T/C (13) | −0.83 (0.84) | | |
| | G/T + C/C (1) | −1.01 (0) | | |
| | T/T + T/C (2) | −1.41 (1.74) | | |
| | T/T + C/C (3) | −0.36 (0.18) | | |

Chi-sq statistic generated from the Non-parametric ANOVA Kruskal-Wallis test

TABLES r1-r3
Net increase in 1 μM LA-419 over control (Individual allelic
combination).
Reference group = G/G + T/T

| | | | | |
|---|---|---|---|---|
| | | Net increase, | | |
| | | nmols/L | Chi-sq | |
| Variable | Level (n) | (mean ± sd) | statistic | p value |

r1)
NO (1 μM LA-419 increase over Control)

| | | | | |
|---|---|---|---|---|
| G894T + T-786C | G/G + T/T (18) | 31.22 (71.86) | 5.6060 | 0.47 |
| variant | G/G + T/C (8) | 5.33 (68.90) | | |
| | G/G/+C/C (0) | — | | |
| | G/T + T/T (4) | 28.92 (13.00) | | |
| | G/T + T/C (13) | 76.26 (75.55) | | |
| | G/T + C/C (1) | −53.67 (0) | | |
| | T/T + T/C (2) | 20.83 (131.76) | | |
| | T/T + C/C (3) | 20.67 (16.77) | | |

r2)
ONOO⁻ (1 μM LA-419 increase over Control)

| | | | | |
|---|---|---|---|---|
| G894T + T-786C | G/G + T/T (18) | −32.54 (86.18) | 10.6444 | 0.10 |
| variant | G/G + T/C (8) | 13.13 (106.46) | | |
| | G/G/+C/C (0) | — | | |
| | G/T + T/T (4) | −2.83 (64.48) | | |
| | G/T + T/C (13) | −81.10 (79.76) | | |
| | G/T + C/C (1) | 228.33 (0) | | |
| | T/T + T/C (2) | −227.83 (117.62) | | |
| | T/T + C/C (3) | −42.44 (9.91) | | |

r3)
NO/ONOO⁻ (1 μM LA-419 increase over Control)

| | | | | |
|---|---|---|---|---|
| G894T + T-786C | G/G + T/T (18) | 1.16 (9.12) | 6.7479 | 0.34 |
| variant | G/G + T/C (8) | −0.88 (0.63) | | |
| | G/G/+C/C (0) | — | | |
| | G/T + T/T (4) | −0.99 (1.06) | | |
| | G/T + T/C (13) | −1.21 (1.06) | | |
| | G/T + C/C (1) | −0.24 (0) | | |
| | T/T + T/C (2) | 0.07 (0.61) | | |
| | T/T + C/C (3) | −0.56 (0.56) | | |

Chi-sq statistic generated from the Non-parametric ANOVA Kruskal-Wallis test

TABLES s1-s3
Net increase in 5 μM LA-419 over control. Comparison between major
allele homozygote and minor allele carriers (reference vs. all carriers
combination).
Reference group = G/G + T/T.
Combination = G/G + T/C, G/G/+C/C, G/T + T/T, G/T + T/C,
G/T + C/C, T/T + T/C, T/T + C/C

| | | | | |
|---|---|---|---|---|
| | | Net increase, nmols/L | t | |
| Variable | Level (n) | (mean ± sd) | statistic | p value |

s1)
NO (5 μM LA-419 increase over Control)

| | | | | |
|---|---|---|---|---|
| Genotype | Reference (18) | 56.59 (19.21) | 1.55 | 0.13 |
| | Combination (31) | 46.78 (22.38) | | |

s2)
ONOO⁻ (5 μM LA-419 increase over Control)

| | | | | |
|---|---|---|---|---|
| Genotype | Reference (18) | −77.59 (31.16) | −0.8088 | 0.42 |
| | Combination (31) | −68.74 (26.87) | | |

s3)
NO/ONOO⁻ (5 μM LA-419 increase over Control)

| | | | | |
|---|---|---|---|---|
| Genotype | Reference (18) | −0.88 (0.54) | −0.9436 | 0.35 |
| | Combination (31) | −0.87 (0.81) | | |

t statistic generated from the Non-parametric Mann-Whitney U test

TABLES t1-t3
Net increase in 1 μM LA-419 over control. Comparison between major
allele homozygote and minor allele carriers (reference vs. all carriers
combination).
Reference group = G/G + T/T.
Combination = G/G + T/C, G/G/+C/C, G/T + T/T, G/T + T/C,
G/T + C/C, T/T + T/C, T/T + C/C

| | | | | |
|---|---|---|---|---|
| | | Net increase, nmols/L | t | |
| Variable | Level (n) | (mean ± sd) | statistic | p value |

t1)
NO (1 μM LA-419 increase over Control)

| | | | | |
|---|---|---|---|---|
| Genotype | Reference (18) | 31.22 (71.86) | 0.3837 | 0.70 |
| | Combination (31) | 38.70 (72.56) | | |

t2)
ONOO⁻ (1 μM LA-419 increase over Control)

| | | | | |
|---|---|---|---|---|
| Genotype | Reference (18) | −32.54 (86.18) | 0.7052 | 0.48 |
| | Combination (31) | −42.43 (111.75) | | |

t3)
NO/ONOO⁻ (1 μM LA-419 increase over Control)

| | | | | |
|---|---|---|---|---|
| Genotype | Reference (18) | 1.16 (9.12) | −0.3422 | 0.73 |
| | Combination (31) | −0.92 (0.90) | | |

t statistic generated from the Non-parametric Mann-Whitney U test

### Percent Change

TABLES u1-u3
% Change - Mean response for all genotypes and races (5 μM LA-419)
% Change = (avg. 5 μM − avg. control)/(avg. control) * 100

| | | | | |
|---|---|---|---|---|
| | | % Change | Chi-sq | |
| Variable | Level (n) | (mean ± sd) | statistic | p value |

u1)
NO (5 μM LA-419)

| | | | | |
|---|---|---|---|---|
| G894T variant | G/G (26) | 15.93 (6.37) | 4.1721 | 0.12 |
| | G/T (18) | 13.43 (6.31) | | |
| | T/T (5) | 11.67 (6.86) | | |
| T-786C variant | T/T (22) | 16.16 (5.97) | 3.0028 | 0.22 |
| | T/C (23) | 13.58 (6.82) | | |
| | C/C (4) | 11.63 (5.99) | | |
| Intron 4 variant | b/b (31) | 13.52 (6.80) | 2.1674 | 0.538 |
| | b/a (10) | 16.81 (6.88) | | |
| | a/a (3) | 13.60 (3.88) | | |
| | c/c (1) | 17.36 (0) | | |
| Race | Asian (1) | 13.97 (0) | 3.2768 | 0.66 |
| | Black (12) | 12.82 (5.82) | | |
| | Caucasian (26) | 14.30 (6.78) | | |
| | Caucasian/ | 16.93 (4.34) | | |
| | Black (2) | | | |
| | Hispanic (6) | 17.23 (7.65) | | |
| | Not Reported (2) | 18.69 (5.53) | | |

u2)
ONOO⁻ (5 μM LA-419)

| | | | | |
|---|---|---|---|---|
| G894T variant | G/G (26) | −20.65 (7.38) | 1.9795 | 0.37 |
| | G/T (18) | −17.85 (6.26) | | |
| | T/T (5) | −19.03 (8.40) | | |
| T-786C variant | T/T (22) | −20.98 (8.19) | 0.9125 | 0.63 |
| | T/C (23) | −17.73 (5.53) | | |
| | C/C (4) | −21.03 (7.77) | | |
| Intron 4 variant | b/b (31) | −19.19 (7.76) | 1.5576 | 0.669 |
| | b/a (10) | −19.70 (5.79) | | |
| | a/a (3) | −16.60 (3.31) | | |
| | c/c (1) | −22.99 (0) | | |
| Race | Asian (1) | −27.79 (0) | 14.2033 | 0.01† |
| | Black (12) | −16.43 (6.22) | | |
| | Caucasian (26) | −17.84 (5.90) | | |
| | Caucasian/ | −25.30 (9.90) | | |
| | Black (2) | | | |
| | Hispanic (6) | −25.17 (5.79) | | |
| | Not Reported (2) | −31.56 (5.34) | | |

u3)
NO/ONOO⁻ (5 μM LA-419)

| | | | | |
|---|---|---|---|---|
| G894T variant | G/G (26) | 47.91 (17.67) | 4.4466 | 0.11 |
| | G/T (18) | 38.86 (13.20) | | |
| | T/T (5) | 39.03 (13.43) | | |
| T-786C variant | T/T (22) | 49.09 (17.76) | 2.9791 | 0.23 |
| | T/C (23) | 38.74 (13.67) | | |
| | C/C (4) | 42.33 (13.21) | | |
| Intron 4 variant | b/b (31) | 42.24 (17.73) | 0.9604 | 0.811 |
| | b/a (10) | 45.81 (14.36) | | |
| | a/a (3) | 36.75 (3.58) | | |
| | c/c (1) | 52.87 (0) | | |
| Race | Asian (1) | 57.83 (0) | 16.6884 | 0.01† |
| | Black (12) | 36.30 (14.59) | | |
| | Caucasian (26) | 39.65 (12.24) | | |
| | Caucasian/ | 58.92 (16.01) | | |
| | Black (2) | | | |
| | Hispanic (6) | 57.79 (12.24) | | |
| | Not Reported (2) | 75.55 (20.43) | | |

Chi-sq statistic generated from the Non-parametric ANOVA Kruskal-Wallis test

TABLES v1-v3
% Change - Mean response for all genotypes and races (1 μM LA-419)
% Change = (avg. 1 μM − avg. control)/(avg. control) * 100

| | | | | |
|---|---|---|---|---|
| | | % Change | Chi-sq | |
| Variable | Level (n) | (mean ± sd) | statistic | p value |

v1)
NO (1 μM LA-419)

| | | | | |
|---|---|---|---|---|
| G894T variant | G/G (26) | 7.77 (18.82) | 0.5639 | 0.75 |
| | G/T (18) | 20.99 (29.98) | | |
| | T/T (5) | 8.69 (18.85) | | |
| T-786C variant | T/T (22) | 9.48 (17.79) | 2.0563 | 0.36 |
| | T/C (23) | 17.70 (29.53) | | |
| | C/C (4) | 1.91 (11.63) | | |
| Intron 4 variant | b/b (31) | 13.46 (23.13) | 4.2767 | 0.233 |
| | b/a (10) | 20.51 (28.08) | | |
| | a/a (3) | −4.57 (24.67) | | |
| | c/c (1) | −26.76 (0) | | |
| Race | Asian (1) | 8.95 (0) | 9.1530 | 0.10 |
| | Black (12) | −4.73 (18.81) | | |
| | Caucasian (26) | 21.25 (25.58) | | |
| | Caucasian/Black (2) | 9.57 (6.43) | | |
| | Hispanic (6) | 11.74 (17.99) | | |
| | Not Reported (2) | 14.50 (2.19) | | |

v2)
ONOO⁻ (1 μM LA-419)

| | | | | |
|---|---|---|---|---|
| G894T variant | G/G (26) | −2.10 (27.33) | 2.8861 | 0.24 |
| | G/T (18) | −9.58 (26.95) | | |
| | T/T (5) | −25.44 (21.34) | | |
| T-786C variant | T/T (22) | −5.17 (21.75) | 3.9697 | 0.14 |
| | T/C (23) | −12.40 (28.22) | | |
| | C/C (4) | 11.13 (43.85) | | |
| Intron 4 variant | b/b (31) | −10.24 (27.17) | 2.5320 | 0.470 |
| | b/a (10) | −6.58 (30.92) | | |
| | a/a (3) | 9.62 (33.98) | | |
| | c/c (1) | −0.63 (0) | | |
| Race | Asian (1) | −18.50 (0) | 15.2522 | 0.01† |
| | Black (12) | 16.25 (36.48) | | |
| | Caucasian (26) | −18.41 (17.30) | | |
| | Caucasian/Black (2) | 9.37 (21.79) | | |
| | Hispanic (6) | −6.29 (19.74) | | |
| | Not Reported (2) | −16.55 (2.66) | | |

v3)
NO/ONOO⁻ (1 μM LA-419)

| | | | | |
|---|---|---|---|---|
| G894T variant | G/G (26) | 21.83 (51.78) | 0.7130 | 0.70 |
| | G/T (18) | 50.45 (77.28) | | |
| | T/T (5) | 61.97 (81.82) | | |
| T-786C variant | T/T (22) | 25.48 (51.97) | 2.6416 | 0.27 |
| | T/C (23) | 52.82 (77.96) | | |
| | C/C (4) | 2.52 (35.64) | | |
| Intron 4 variant | b/b (31) | 43.27 (71.09) | 3.4675 | 0.325 |
| | b/a (10) | 42.92 (65.15) | | |
| | a/a (3) | −3.62 (44.56) | | |
| | c/c (1) | −26.15 (0) | | |
| Race | Asian (1) | 33.68 (0) | 15.0458 | 0.01† |
| | Black (12) | −8.17 (36.37) | | |
| | Caucasian (26) | 61.80 (74.46) | | |
| | Caucasian/Black (2) | 3.77 (26.27) | | |
| | Hispanic (6) | 26.85 (45.85) | | |
| | Not Reported (2) | 37.24 (1.75) | | |

Chi-sq statistic generated from the Non-parametric ANOVA Kruskal-Wallis test

TABLE x1
Ratio of % Change NO/% Change ONOO⁻ (5 μM LA-419):
% Change NO/% Change ONOO⁻ (5 μM LA-419)

| | | % Change Ratio | Chi-sq | |
|---|---|---|---|---|
| Variable | Level (n) | (mean ± sd) | statistic | p value |
| G894T | G/G (26) | −0.82 (0.40) | 0.5943 | 0.74 |
| variant | G/T (18) | −0.88 (0.60) | | |
| | T/T (5) | −0.80 (0.73) | | |
| T-786C | T/T (22) | −0.90 (0.47) | 1.2795 | 0.53 |
| variant | T/C (23) | −0.81 (0.56) | | |
| | C/C (4) | −0.62 (0.39) | | |
| Race | Asian (1) | −0.50 (0) | 1.9471 | 0.86 |
| | Black (12) | −0.79 (0.44) | | |
| | Caucasian (26) | −0.92 (0.59) | | |
| | Caucasian/Black (2) | −0.76 (0.47) | | |
| | Hispanic (6) | −0.73 (0.40) | | |
| | Not Reported (2) | −0.59 (0.08) | | |

Chi-sq statistic generated from the Non-parametric ANOVA Kruskal-Wallis test

TABLE x2
Ratio of % Change NO/% Change ONOO⁻ (1 μM LA-419):
% Change NO/% Change ONOO⁻ (1 μM LA-419)

| | | % Change Ratio | Chi-sq | |
|---|---|---|---|---|
| Variable | Level (n) | (mean ± sd) | statistic | p value |
| G894T | G/G (26) | 0.76 (8.64) | 1.8736 | 0.39 |
| variant | G/T (18) | −1.15 (0.91) | | |
| | T/T (5) | −0.66 (1.02) | | |
| T-786C | T/T (22) | 1.06 (9.40) | 0.9272 | 0.63 |
| variant | T/C (23) | −1.06 (0.82) | | |
| | C/C (4) | −0.80 (1.04) | | |
| Race | Asian (1) | −0.48 (0) | 2.2419 | 0.81 |
| | Black (12) | 2.81 (12.60) | | |
| | Caucasian (26) | −1.09 (1.30) | | |
| | Caucasian/Black (2) | −1.07 (1.80) | | |
| | Hispanic (6) | −0.90 (0.55) | | |
| | Not Reported (2) | −0.90 (0.28) | | |

Chi-sq statistic generated from the Non-parametric ANOVA Kruskal-Wallis test

### Fold Difference

TABLES y1-y3
Fold difference LA-419 5 μM - comparison between genotypes
Fold difference = (LA-419 5 μM)/(control)

| | | | | |
|---|---|---|---|---|
| | | Fold difference | Chi-sq | |
| Variable | Level (n) | (mean ± sd) | statistic | p value |

y1)
NO (LA-419 5 μM)

| | | | | |
|---|---|---|---|---|
| G894T variant | G/G (26) | 1.16 (0.06) | 4.1721 | 0.12 |
| | G/T (18) | 1.13 (0.06) | | |
| | T/T (5) | 1.12 (0.07) | | |
| T-786C variant | T/T (22) | 1.16 (0.06) | 3.0028 | 0.22 |
| | T/C (23) | 1.14 (0.07) | | |
| | C/C (4) | 1.12 (0.06) | | |
| Intron 4 variant | b/b (31) | 1.14 (0.07) | 2.1674 | 0.538 |
| | b/a (10) | 1.17 (0.07) | | |
| | a/a (3) | 1.14 (0.04) | | |
| | c/c (1) | 1.17 (0) | | |

y2)
ONOO⁻ (LA-419 5 μM)

| | | | | |
|---|---|---|---|---|
| G894T variant | G/G (26) | 0.79 (0.07) | 1.9795 | 0.37 |
| | G/T (18) | 0.82 (0.06) | | |
| | T/T (5) | 0.81 (0.08) | | |
| T-786C variant | T/T (22) | 0.79 (0.08) | 0.9125 | 0.63 |
| | T/C (23) | 0.82 (0.06) | | |
| | C/C (4) | 0.79 (0.08) | | |
| Intron 4 variant | b/b (31) | 0.81 (0.08) | 1.5576 | 0.669 |
| | b/a (10) | 0.80 (0.06) | | |
| | a/a (3) | 0.83 (0.03) | | |
| | c/c (1) | 0.77 (0) | | |

y3)
NO/ONOO⁻ (LA-419 5 μM)

| | | | | |
|---|---|---|---|---|
| G894T variant | G/G (26) | 1.48 (0.18) | 4.4466 | 0.108 |
| | G/T (18) | 1.39 (0.13) | | |
| | T/T (5) | 1.39 (0.13) | | |
| T-786C variant | T/T (22) | 1.49 (0.18) | 2.9791 | 0.226 |
| | T/C (23) | 1.39 (0.14) | | |
| | C/C (4) | 1.42 (0.13) | | |
| Intron 4 variant | b/b (31) | 1.42 (0.18) | 0.9604 | 0.811 |
| | b/a (10) | 1.46 (0.14) | | |
| | a/a (3) | 1.37 (0.04) | | |
| | c/c (1) | 1.53 (0) | | |

Fold difference = avg 5 μM/avg Cntl. Chi-sq statistic generated from the Non-parametric ANOVA Kruskal-Wallis test

TABLES z1-z3
Fold difference LA-419 1 μM - comparison between genotypes
Fold difference = (LA-419 1 μM)/(control)

| | | | | |
|---|---|---|---|---|
| | | Fold difference | Chi-sq | |
| Variable | Level (n) | (mean ± sd) | statistic | p value |

z1)
NO (LA-419 1 μM)

| | | | | |
|---|---|---|---|---|
| G894T variant | G/G (26) | 1.08 (0.19) | 0.5639 | 0.75 |
| | G/T (18) | 1.21 (0.30) | | |
| | T/T (5) | 1.09 (0.19) | | |
| T-786C variant | T/T (22) | 1.09 (0.18) | 2.0563 | 0.36 |
| | T/C (23) | 1.18 (0.30) | | |
| | C/C (4) | 1.02 (0.12) | | |
| Intron 4 variant | b/b (31) | 1.13 (0.23) | 4.276 | 0.233 |
| | b/a (10) | 1.21 (0.28) | | |
| | a/a (3) | 0.95 (0.25) | | |
| | c/c (1) | 0.73 (0) | | |

z2)
ONOO⁻ (LA-419 1 μM)

| | | | | |
|---|---|---|---|---|
| G894T variant | G/G (26) | 0.98 (0.27) | 2.8861 | 0.24 |
| | G/T (18) | 0.90 (0.27) | | |
| | T/T (5) | 0.75 (0.21) | | |
| T-786C variant | T/T (22) | 0.95 (0.22) | 3.9697 | 0.14 |
| | T/C (23) | 0.88 (0.28) | | |
| | C/C (4) | 1.11 (0.44) | | |
| Intron 4 variant | b/b (31) | 0.90 (0.27) | 2.5320 | 0.470 |
| | b/a (10) | 0.93 (0.31) | | |
| | a/a (3) | 1.10 (0.34) | | |
| | c/c (1) | 0.99 (0) | | |

z3)
NO/ONOO⁻ (LA-419 1 μM)

| | | | | |
|---|---|---|---|---|
| G894T variant | G/G (26) | 1.22 (0.52) | 0.7130 | 0.700 |
| | G/T (18) | 1.50 (0.77) | | |
| | T/T (5) | 1.62 (0.82) | | |
| T-786C variant | T/T (22) | 1.25 (0.52) | 2.6416 | 0.267 |
| | T/C (23) | 1.53 (0.78) | | |
| | C/C (4) | 1.03 (0.36) | | |
| Intron 4 variant | b/b (31) | 1.43 (0.71) | 3.4675 | 0.325 |
| | b/a (10) | 1.43 (0.65) | | |
| | a/a (3) | 0.96 (0.45) | | |
| | c/c (1) | 0.74 (0) | | |

Fold difference = avg 1 μM/avg Cntl. Chi-sq statistic generated from the Non-parametric ANOVA Kruskal-Wallis test

TABLES aa1-aa3
Fold difference LA-419 5 μM - comparison between major allele
homozygotes and minor allele carriers
Fold difference = (LA-419 5 μM)/(control)

| | | | | |
|---|---|---|---|---|
| | | Fold difference | | |
| Variable | Level (n) | (mean ± sd) | t statistic | p value |

aa1)
NO (LA-419 5 μM)

| | | | | |
|---|---|---|---|---|
| G894T variant | G/G (26) | 1.16 (0.06) | −1.9933 | 0.05† |
| | G/T & T/T (23) | 1.13 (0.06) | | |
| T-786C variant | T/T (22) | 1.16 (0.06) | 1.5779 | 0.12 |
| | T/C & C/C (27) | 1.13 (0.07) | | |
| Intron 4 variant | b/b (31) | 1.14 (0.07) | 1.2136 | 0.231 |
| | b/a, a/a, c/c (14) | 1.16 (0.06) | | |

aa2)
ONOO⁻ (LA-419 5 μM)

| | | | | |
|---|---|---|---|---|
| G894T variant | G/G (26) | 0.79 (0.07) | 1.3923 | 0.17 |
| | G/T & T/T (23) | 0.82 (0.07) | | |
| T-786C variant | T/T (22) | 0.79 (0.08) | −0.8744 | 0.39 |
| | T/C & C/C (27) | 0.82 (0.06) | | |
| Intron 4 variant | b/b (31) | 0.81 (0.08) | −0.3555 | 0.724 |
| | b/a, a/a, c/c (14) | 0.81 (0.05) | | |

aa3)
NO/ONOO⁻ (LA-419 5 μM)

| | | | | |
|---|---|---|---|---|
| G894T variant | G/G (26) | 1.48 (0.18) | −2.0935 | 0.042† |
| | G/T & T/T (23) | 1.39 (0.13) | | |
| T-786C variant | T/T (22) | 1.49 (0.18) | 1.6784 | 0.100 |
| | T/C & C/C (27) | 1.39 (0.13) | | |
| Intron 4 variant | b/b (31) | 1.42 (0.18) | 0.5516 | 0.584 |
| | b/a, a/a, c/c (14) | 1.44 (0.13) | | |

Fold difference = avg 5 μM/avg Cntl. t statistic generated from the Non-parametric Mann-Whitney U test

TABLES bb1-bb3
Fold difference LA-419 1 μM - comparison between major allele
homozygotes and minor allele carriers
Fold change = (LA-419 1 μM)/(control)

| | | | | |
|---|---|---|---|---|
| | | Fold difference | | |
| Variable | Level (n) | (mean ± sd) | t statistic | p value |

bb1)
NO (LA-419 1 μM)

| | | | | |
|---|---|---|---|---|
| G894T variant | G/G (26) | 1.08 (0.19) | 0.0100 | 0.99 |
| | G/T & T/T (23) | 1.18 (0.28) | | |
| T-786C variant | T/T (22) | 1.09 (0.18) | 0.2111 | 0.83 |
| | T/C & C/C (27) | 1.15 (0.28) | | |
| Intron 4 variant | b/b (31) | 1.13 (0.23) | 0.0000 | 1.000 |
| | b/a, a/a, c/c (14) | 1.13 (0.23) | | |

bb2)
ONOO⁻ (LA-419 1 μM)

| | | | | |
|---|---|---|---|---|
| G894T variant | G/G (26) | 0.98 (0.27) | −1.3322 | 0.19 |
| | G/T & T/T (23) | 0.87 (0.26) | | |
| T-786C variant | T/T (22) | 0.95 (0.22) | 1.4774 | 0.15 |
| | T/C & C/C (27) | 0.91 (0.31) | | |
| Intron 4 variant | b/b (31) | 0.90 (0.27) | 0.7233 | 0.473 |
| | b/a, a/a, c/c (14) | 0.97 (0.30) | | |

bb3)
NO/ONOO⁻ (LA-419 1 μM)

| | | | | |
|---|---|---|---|---|
| G894T variant | G/G (26) | 1.22 (0.52) | 0.4508 | 0.654 |
| | G/T & T/T (23) | 1.53 (0.77) | | |
| T-786C variant | T/T (22) | 1.25 (0.52) | −0.6131 | 0.543 |
| | T/C & C/C (27) | 1.45 (0.75) | | |
| Intron 4 variant | b/b (31) | 1.43 (0.71) | −0.4781 | 0.635 |
| | b/a, a/a, c/c (14) | 1.28 (0.62) | | |

Fold difference = avg 1 μM/avg Cntl. t statistic generated from the Non-parametric Mann-Whitney U test

TABLES cc1-cc3
Fold difference LA-419 5 μM (Individual allelic combination).
Reference group = G/G + T/T
Fold difference = (LA-419 5 μM)/(control)

| | | | | |
|---|---|---|---|---|
| | | Fold difference | Chi-sq | |
| Variable | Level (n) | (mean ± sd) | statistic | p value |

cc1)
NO (5 μM LA-419)

| | | | | |
|---|---|---|---|---|
| G894T + T-786C | G/G + T/T (18) | 1.17 (0.06) | 4.6351 | 0.59 |
| variant | G/G + T/C (8) | 1.14 (0.07) | | |
| | G/G/+C/C (0) | — | | |
| | G/T + T/T (4) | 1.14 (0.05) | | |
| | G/T + T/C (13) | 1.13 (0.07) | | |
| | G/T + C/C (1) | 1.12 (0) | | |
| | T/T + T/C (2) | 1.12 (0.09) | | |
| | T/T + C/C (3) | 1.12 (0.07) | | |

cc2)
ONOO⁻ (5 μM LA-419)

| | | | | |
|---|---|---|---|---|
| G894T + T-786C | G/G + T/T (18) | 0.79 (0.08) | 4.4377 | 0.62 |
| variant | G/G + T/C (8) | 0.80 (0.07) | | |
| | G/G/+C/C (0) | — | | |
| | G/T + T/T (4) | 0.79 (0.12) | | |
| | G/T + T/C (13) | 0.83 (0.04) | | |
| | G/T + C/C (1) | 0.85 (0) | | |
| | T/T + T/C (2) | 0.87 (0.06) | | |
| | T/T + C/C (3) | 0.77 (0.08) | | |

cc3)
NO/ONOO⁻ (5 μM LA-419)

| | | | | |
|---|---|---|---|---|
| G894T + T-786C | G/G + T/T (18) | 1.50 (0.18) | 8.3116 | 0.216 |
| variant | G/G + T/C (8) | 1.44 (0.18) | | |
| | G/G/+C/C (0) | — | | |
| | G/T + T/T (4) | 1.47 (0.19) | | |
| | G/T + T/C (13) | 1.37 (0.11) | | |
| | G/T + C/C (1) | 1.32 (0) | | |
| | T/T + T/C (2) | 1.29 (0.01) | | |
| | T/T + C/C (3) | 1.46 (0.14) | | |

Chi-sq statistic generated from the Non-parametric ANOVA Kruskal-Wallis test

TABLES dd1-dd3
Fold difference LA-419 1 μM (Individual allelic combination).
Reference group = G/G + T/T
Fold difference = (LA-419 1 μM)/(control)

| | | | | |
|---|---|---|---|---|
| | | Fold difference | Chi-sq | |
| Variable | Level (n) | (mean ± sd) | statistic | p value |

dd1)
NO (1 μM LA-419)

| | | | | |
|---|---|---|---|---|
| G894T + T-786C | G/G + T/T (18) | 1.10 (0.20) | 5.0913 | 0.53 |
| variant | G/G + T/C (8) | 1.03 (0.17) | | |
| | G/G/+C/C (0) | — | | |
| | G/T + T/T (4) | 1.08 (0.03) | | |
| | G/T + T/C (13) | 1.28 (0.33) | | |
| | G/T + C/C (1) | 0.86 (0) | | |
| | T/T + T/C (2) | 1.11 (0.36) | | |
| | T/T + C/C (3) | 1.07 (0.06) | | |

dd2)
ONOO⁻ (1 μM LA-419)

| | | | | |
|---|---|---|---|---|
| G894T + T-786C | G/G + T/T (18) | 0.94 (0.23) | 11.1807 | 0.08 |
| variant | G/G + T/C (8) | 1.07 (0.36) | | |
| | G/G/+C/C (0) | — | | |
| | G/T + T/T (4) | 0.98 (0.18) | | |
| | G/T + T/C (13) | 0.81 (0.15) | | |
| | G/T + C/C (1) | 1.77 (0) | | |
| | T/T + T/C (2) | 0.52 (0.11) | | |
| | T/T + C/C (3) | 0.89 (0.06) | | |

dd3)
NO/ONOO⁻ (1 μM LA-419)

| | | | | |
|---|---|---|---|---|
| G894T + T-786C | G/G + T/T (18) | 1.28 (0.57) | 8.1332 | 0.229 |
| variant | G/G + T/C (8) | 1.07 (0.37) | | |
| | G/G/+C/C (0) | — | | |
| | G/T + T/T (4) | 1.12 (0.18) | | |
| | G/T + T/C (13) | 1.70 (0.81) | | |
| | G/T + C/C (1) | 0.49 (0) | | |
| | T/T + T/C (2) | 2.25 (1.17) | | |
| | T/T + C/C (3) | 1.20 (0.04) | | |

Chi-sq statistic generated from the Non-parametric ANOVA Kruskal-Wallis test

TABLES ee1-ee3
Fold difference LA-419 5 μM. Comparison between major allele
homozygote and minor allele carriers (reference vs. all carriers
combination).
Reference group = G/G + T/T.
Combination = G/G + T/C, G/G/+C/C, G/T + T/T, G/T + T/C,
G/T + C/C, T/T + T/C, T/T + C/C
Fold difference = (LA-419 5 μM)/(control)

| | | | | |
|---|---|---|---|---|
| | | Fold difference | | |
| Variable | Level (n) | (mean ± sd) | t statistic | p value |

ee1
NO (5 μM LA-419)

| | | | | |
|---|---|---|---|---|
| Genotype | Reference (18) | 1.17 (0.06) | 1.7317 | 0.09 |
| | Combination (31) | 1.13 (0.06) | | |

ee2)
ONOO⁻ (5 μM LA-419)

| | | | | |
|---|---|---|---|---|
| Genotype | Reference (18) | 0.79 (0.06) | −0.8607 | 0.39 |
| | Combination (31) | 0.81 (0.07) | | |

ee3)
NO/ONOO⁻ (5 μM LA-419)

| | | | | |
|---|---|---|---|---|
| Genotype | Reference (18) | 1.50 (0.18) | 1.5451 | 0.130 |
| | Combination (31) | 1.40 (0.14) | | |

t statistic generated from the Non-parametric Mann-Whitney U test

TABLES ff1-ff3
Fold difference LA-419 1 μM. Comparison between major allele
homozygote and minor allele carriers (reference vs. all carriers
combination).
Reference group = G/G + T/T.
Combination = G/G + T/C, G/G/+C/C, G/T + T/T, G/T + T/C,
G/T + C/C, T/T + T/C, T/T + C/C
Fold difference = (LA-419 1 μM)/(control)

| | | | | |
|---|---|---|---|---|
| | | Fold difference | | |
| Variable | Level (n) | (mean ± sd) | t statistic | p value |

ff1)
NO (1 μM LA-419)

| | | | | |
|---|---|---|---|---|
| Genotype | Reference (18) | 1.10 (0.20) | 0.5911 | 0.56 |
| | Combination (31) | 1.14 (0.26) | | |

ff2)
ONOO⁻ (1 μM LA-419)

| | | | | |
|---|---|---|---|---|
| Genotype | Reference (18) | 0.94 (0.23) | 0.9851 | 0.33 |
| | Combination (31) | 0.92 (0.30) | | |

ff3)
NO/ONOO⁻ (1 μM LA-419)

| | | | | |
|---|---|---|---|---|
| Genotype | Reference (18) | 1.28 (0.57) | −0.0726 | 0.942 |
| | Combination (31) | 1.41 (0.71) | | |

t statistic generated from the Non-parametric Mann-Whitney U test

Fold Difference within Caucasian

TABLES gg1-gg3
Caucasian Fold difference LA-419 5 μM - comparison between genotypes
Fold difference = (LA-419 5 μM)/(control)

| | | | | |
|---|---|---|---|---|
| | | Fold difference | Chi-sq | |
| Variable | Level (n) | (mean ± sd) | statistic | p value |

gg1)
NO (LA-419 5 μM) Caucasian

| | | | | |
|---|---|---|---|---|
| G894T variant | G/G (10) | 1.19 (0.07) | 7.8918 | 0.02† |
| | G/T (13) | 1.12 (0.05) | | |
| | T/T (3) | 1.11 (0.07) | | |
| T-786C variant | T/T (10) | 1.17 (0.08) | 3.7379 | 0.15 |
| | T/C (15) | 1.13 (0.06) | | |
| | C/C (1) | 1.08 (0) | | |
| Intron 4 variant | b/b (18) | 1.13 (0.07) | 1.1544 | 0.56 |
| | b/a (5) | 1.16 (0.06) | | |
| | a/a (1) | 1.17 (0) | | |

gg2)
ONOO⁻ (LA-419 5 μM) Caucasian

| | | | | |
|---|---|---|---|---|
| G894T variant | G/G (10) | 0.81 (0.04) | 1.6319 | 0.44 |
| | G/T (13) | 0.83 (0.06) | | |
| | T/T (3) | 0.80 (0.12) | | |
| T-786C variant | T/T (10) | 0.82 (0.07) | 3.2593 | 0.20 |
| | T/C (15) | 0.83 (0.04) | | |
| | C/C (1) | 0.67 (0) | | |
| Intron 4 variant | b/b (18) | 0.82 (0.06) | 0.4844 | 0.78 |
| | b/a (5) | 0.83 (0.05) | | |
| | a/a (1) | 0.85 (0) | | |

gg3)
NO/ONOO− (LA-419 5 μM) Caucasian

| | | | | |
|---|---|---|---|---|
| G894T variant | G/G (10) | 1.47 (0.12) | 5.6513 | 0.06 |
| | G/T (13) | 1.34 (0.09) | | |
| | T/T (3) | 1.39 (0.17) | | |
| T-786C variant | T/T (10) | 1.49 (0.14) | 5.1960 | 0.07 |
| | T/C (15) | 1.35 (0.09) | | |
| | C/C (1) | 1.59 (0) | | |
| Intron 4 variant | b/b (18) | 1.39 (0.13) | 0.1518 | 0.93 |
| | b/a (5) | 1.39 (0.12) | | |
| | a/a (1) | 1.39 (0) | | |

% Control = avg 5 μM/avg Cntl. Chi-sq statistic generated from the Non-parametric ANOVA Kruskal-Wallis test
Fold difference = avg 5 μM/avg Cntl. Chi-sq statistic generated from the Non-parametric ANOVA Kruskal-Wallis test

TABLES hh1-hh3
Caucasian Fold difference LA-419 1 μM - comparison between genotypes
Fold difference = (LA-419 1 μM)/(control)

| | | | | |
|---|---|---|---|---|
| | | Fold difference | Chi-sq | |
| Variable | Level (n) | (mean ± sd) | statistic | p value |

hh1)
NO (LA-419 1 μM) Caucasian

| | | | | |
|---|---|---|---|---|
| G894T variant | G/G (10) | 1.21 (0.12) | 3.6332 | 0.16 |
| | G/T (13) | 1.24 (0.33) | | |
| | T/T (3) | 1.08 (0.26) | | |
| T-786C variant | T/T (10) | 1.20 (0.13) | 3.1174 | 0.21 |
| | T/C (15) | 1.24 (0.32) | | |
| | C/C (1) | 1.02 (0) | | |
| Intron 4 variant | b/b (18) | 1.21 (0.25) | 0.4540 | 0.80 |
| | b/a (5) | 1.28 (0.35) | | |
| | a/a (1) | 1.09 (0) | | |

hh2)
ONOO (LA-419 1 μM) Caucasian

| | | | | |
|---|---|---|---|---|
| G894T variant | G/G (10) | 0.86 (0.18) | 5.1185 | 0.08 |
| | G/T (13) | 0.83 (0.15) | | |
| | T/T (3) | 0.63 (0.19) | | |
| T-786C variant | T/T (10) | 0.86 (0.18) | 2.5840 | 0.27 |
| | T/C (15) | 0.79 (0.17) | | |
| | C/C (1) | 0.83 (0) | | |
| Intron 4 variant | b/b (18) | 0.79 (0.20) | 0.5404 | 0.76 |
| | b/a (5) | 0.85 (0.12) | | |
| | a/a (1) | 0.89 (0) | | |

hh3)
NO/ONOO⁻ (LA-419 1 μM) Caucasians

| | | | | |
|---|---|---|---|---|
| G894T variant | G/G (10) | 1.52 (0.61) | 2.7557 | 0.25 |
| | G/T (13) | 1.63 (0.82) | | |
| | T/T (3) | 1.90 (1.02) | | |
| T-786C variant | T/T (10) | 1.50 (0.62) | 0.1225 | 0.94 |
| | T/C (15) | 1.72 (0.84) | | |
| | C/C (1) | 1.23 (0) | | |
| Intron 4 variant | b/b (18) | 1.69 (0.80) | 0.0640 | 0.97 |
| | b/a (5) | 1.58 (0.77) | | |
| | a/a (1) | 1.23 (0) | | |

Fold difference = avg 1 μM/avg Cntl. Chi-sq statistic generated from the Non-parametric ANOVA Kruskal-Wallis test

TABLES ii1-ii3
Caucasian Fold difference LA-419 5 μM - comparison between major
allele homozygotes and minor allele carriers
Fold difference = (LA-419 5 μM)/(control)

| | | | | |
|---|---|---|---|---|
| | | Fold difference | | |
| Variable | Level (n) | (mean ± sd) | t statistic | p value |

ii1)
NO (5 μM) Caucasian

| | | | | |
|---|---|---|---|---|
| G894T variant | G/G (10) | 1.19 (0.07) | 2.7670 | 0.01† |
| | G/T & T/T (16) | 1.11 (0.05) | | |
| T-786C variant | T/T (10) | 1.17 (0.08) | 1.7656 | 0.09 |
| | T/C & C/C (16) | 1.12 (0.06) | | |
| Intron 4 variant | b/b (18) | 1.13 (0.07) | 1.0333 | 0.31 |
| | b/a & a/a (6) | 1.16 (0.06) | | |

ii2)
ONOO⁻ (5 μM) Caucasian

| | | | | |
|---|---|---|---|---|
| G894T variant | G/G (10) | 0.81 (0.04) | −1.1859 | 0.25 |
| | G/T & T/T (16) | 0.83 (0.07) | | |
| T-786C variant | T/T (10) | 0.82 (0.07) | −0.3953 | 0.67 |
| | T/C & C/C (16) | 0.82 (0.06) | | |
| Intron 4 variant | b/b (18) | 0.82 (0.06) | 0.4333 | 0.67 |
| | b/a & a/a (6) | 0.83 (0.05) | | |

ii3)
NO/ONOO⁻ (5 μM) Caucasian

| | | | | |
|---|---|---|---|---|
| G894T variant | G/G (10) | 1.47 (0.12) | 2.3454 | 0.027† |
| | G/T & T/T (16) | 1.35 (0.10) | | |
| T-786C variant | T/T (10) | 1.45 (0.14) | 1.3967 | 0.17 |
| | T/C & C/C (16) | 1.36 (0.10) | | |
| Intron 4 variant | b/b (18) | 1.39 (0.13) | 0.2333 | 0.82 |
| | b/a & a/a (6) | 1.39 (0.11) | | |

Fold difference = avg 5 μM/avg Cntl. t statistic generated from the Non-parametric Mann-Whitney U test

TABLES jj1-jj3
Caucasian Fold difference LA-419 1 μM - comparison
between major allele homozygotes and minor allele carriers
Fold difference = (LA-419 1 μM)/(control)

| | | | | |
|---|---|---|---|---|
| | | Fold difference | | |
| Variable | Level (n) | (mean ± sd) | t statistic | p value |

jj1)
NO (1 μM) Caucasian

| | | | | |
|---|---|---|---|---|
| G894T variant | G/G (10) | 1.21 (0.12) | 1.7129 | 0.10 |
| | G/T & T/T (16) | 1.21 (0.32) | | |
| T-786C variant | T/T (10) | 1.20 (0.13) | 1.0804 | 0.29 |
| | T/C & C/C (16) | 1.22 (0.31) | | |
| Intron 4 variant | b/b (18) | 1.21 (0.25) | 0.3667 | 0.72 |
| | b/a & a/a (6) | 1.25 (0.33) | | |

jj2)
ONOO⁻ (1 μM) Caucasian

| | | | | |
|---|---|---|---|---|
| G894T variant | G/G (10) | 0.86 (0.18) | 1.0804 | 0.29 |
| | G/T & T/T (16) | 0.79 (0.17) | | |
| T-786C variant | T/T (10) | 0.86 (0.18) | 1.5021 | 0.15 |
| | T/C & C/C (16) | 0.79 (0.17) | | |
| Intron 4 variant | b/b (18) | 0.79 (0.20) | 0.7000 | 0.49 |
| | b/a & a/a (6) | 0.86 (0.10) | | |

jj3)
NO/ONOO⁻ (1 μM) Caucasian

| | | | | |
|---|---|---|---|---|
| G894T variant | G/G (10) | 1.52 (0.61) | 0.8696 | 0.39 |
| | G/T & T/T (16) | 1.68 (0.83) | | |
| T-786C variant | T/T (10) | 1.50 (0.62) | −0.0264 | 0.98 |
| | T/C & C/C (16) | 1.69 (0.82) | | |
| Intron 4 variant | b/b (18) | 1.69 (0.80) | −0.1667 | 0.87 |
| | b/a & a/a (6) | 1.52 (0.70) | | |

Fold difference = avg 1 μM/avg Cntl. t statistic generated from the Non-parametric Mann-Whitney U test

TABLES kk1-kk3
Caucasian Fold difference LA-419 5 μM (Individual allelic
combination). Reference group = G/G + T/T
Fold difference = (LA-419 5 μM)/(control)

| | | | | |
|---|---|---|---|---|
| | | Fold difference | Chi-sq | |
| Variable | Level (n) | (mean ± sd) | statistic | p value |

kk1)
NO (5 μM LA-419) Caucasian

| | | | | |
|---|---|---|---|---|
| G894T + T-786C | G/G + T/T (7) | 1.19 (0.08) | 8.9129 | 0.11 |
| variant | G/G + T/C (3) | 1.19 (0.02) | | |
| | G/G/+C/C (0) | — | | |
| | G/T + T/T (3) | 1.14 (0.06) | | |
| | G/T + T/C (10) | 1.11 (0.05) | | |
| | G/T + C/C (0) | — | | |
| | T/T + T/C (2) | 1.12 (0.09) | | |
| | T/T + C/C (1) | 1.08 (0) | | |

kk2)
ONOO⁻ (5 μM LA-419) Caucasian

| | | | | |
|---|---|---|---|---|
| G894T + T-786C | G/G + T/T (7) | 0.81 (0.04) | 5.6036 | 0.35 |
| variant | G/G + T/C (3) | 0.81 (0.03) | | |
| | G/G/+C/C (0) | — | | |
| | G/T + T/T (3) | 0.83 (0.11) | | |
| | G/T + T/C (10) | 0.84 (0.04) | | |
| | G/T + C/C (0) | — | | |
| | T/T + T/C (2) | 0.87 (0.06) | | |
| | T/T + C/C (1) | 0.67 (0) | | |

kk3)
NO/ONOO⁻ (5 μM LA-419) Caucasian

| | | | | |
|---|---|---|---|---|
| G894T + T-786C | G/G + T/T (7) | 1.47 (0.14) | 11.0915 | 0.0496† |
| variant | G/G + T/C (3) | 1.47 (0.08) | | |
| | G/G/+C/C (0) | — | | |
| | G/T + T/T (3) | 1.39 (0.14) | | |
| | G/T + T/C (10) | 1.33 (0.06) | | |
| | G/T + C/C (0) | — | | |
| | T/T + T/C (2) | 1.29 (0.01) | | |
| | T/T + C/C (1) | 1.59 (0) | | |

Chi-sq statistic generated from the Non-parametric ANOVA Kruskal-Wallis test

TABLES ll1-ll3
Caucasian Fold difference LA-419 1 μM (Individual allelic
combination). Reference group = G/G + T/T
Fold difference = (LA-419 1 μM)/(control)

| | | | | |
|---|---|---|---|---|
| | | Fold difference | Chi-sq | |
| Variable | Level (n) | (mean ± sd) | statistic | p value |

ll1)
NO (1 μM LA-419) Caucasian

| | | | | |
|---|---|---|---|---|
| G894T + T-786C | G/G + T/T (7) | 1.25 (0.12) | 5.7933 | 0.33 |
| variant | G/G + T/C (3) | 1.13 (0.03) | | |
| | G/G/+C/C (0) | — | | |
| | G/T + T/T (3) | 1.08 (0.03) | | |
| | G/T + T/C (10) | 1.29 (0.37) | | |
| | G/T + C/C (0) | — | | |
| | T/T + T/C (2) | 1.11 (0.36) | | |
| | T/T + C/C (1) | 1.02 (0) | | |

ll2)
ONOO⁻ (1 μM LA-419) Caucasian

| | | | | |
|---|---|---|---|---|
| G894T + T-786C | G/G + T/T (7) | 0.84 (0.22) | 6.2678 | 0.28 |
| variant | G/G + T/C (3) | 0.89 (0.02) | | |
| | G/G/+C/C (0) | — | | |
| | G/T + T/T (3) | 0.90 (0.06) | | |
| | G/T + T/C (10) | 0.81 (0.16) | | |
| | G/T + C/C (0) | — | | |
| | T/T + T/C (2) | 0.52 (0.11) | | |
| | T/T + C/C (1) | 0.83 (0) | | |

ll3)
NO/ONOO⁻ (1 μM LA-419) Caucasian

| | | | | |
|---|---|---|---|---|
| G894T + T-786C | G/G + T/T (7) | 1.63 (0.72) | 4.3243 | 0.50 |
| variant | G/G + T/C (3) | 1.27 (0.04) | | |
| | G/G/+C/C (0) | — | | |
| | G/T + T/T (3) | 1.21 (0.04) | | |
| | G/T + T/C (10) | 1.75 (0.91) | | |
| | G/T + C/C (0) | — | | |
| | T/T + T/C (2) | 2.25 (1.17) | | |
| | T/T + C/C (1) | 1.23 (0) | | |

Chi-sq statistic generated from the Non-parametric ANOVA Kruskal-Wallis test

TABLES mm1-mm3
Caucasian Fold difference LA-419 5 μM. Comparison
between major allele homozygote and minor allele carriers
(reference vs. all carriers combination).
Reference group = G/G + T/T.
Combination = G/G + T/C, G/G/+C/C, G/T + T/T,
G/T + T/C, G/T + C/C, T/T + T/C, T/T + C/C
Fold difference = (LA-419 5 μM)/(control)

| | | | | |
|---|---|---|---|---|
| | | Fold difference | | |
| Variable | Level (n) | (mean ± sd) | t statistic | p value |

mm1)
NO (5 μM LA-419) Caucasian

| | | | | |
|---|---|---|---|---|
| Genotype | Reference (7) | 1.19 (0.08) | 1.9076 | 0.068 |
| | Combination (19) | 1.13 (0.06) | | |

mm2)
ONOO⁻ (5 μM LA-419) Caucasian

| | | | | |
|---|---|---|---|---|
| Genotype | Reference (7) | 0.81 (0.04) | −0.9249 | 0.36 |
| | Combination (19) | 0.83 (0.06) | | |

mm3)
NO/ONOO⁻ (5 μM LA-419) Caucasian

| | | | | |
|---|---|---|---|---|
| Genotype | Reference (7) | 1.47 (0.14) | 1.5608 | 0.13 |
| | Combination (19) | 1.37 (0.10) | | |

t statistic generated from the Non-parametric Mann-Whitney U test

TABLES nn1-nn3
Caucasian Fold difference LA-419 1 μM. Comparison between
major allele homozygote and minor allele carriers (reference vs. all
carriers combination).
Reference group = G/G + T/T.
Combination = G/G + T/C, G/G/+C/C, G/T + T/T,
G/T + T/C, G/T + C/C, T/T + T/C, T/T + C/C
Fold difference = (LA-419 1 μM)/(control)

| | | | | |
|---|---|---|---|---|
| | | Fold difference | | |
| Variable | Level (n) | (mean ± sd) | t statistic | p value |

nn1)
NO (1 μM LA-419) Caucasian

| | | | | |
|---|---|---|---|---|
| Genotype | Reference (7) | 1.25 (0.12) | 1.8498 | 0.076 |
| | Combination (19) | 1.20 (0.29) | | |

nn2)
ONOO⁻ (1 μM LA-419) Caucasian

| | | | | |
|---|---|---|---|---|
| Genotype | Reference (7) | 0.84 (0.22) | 0.9827 | 0.34 |
| | Combination (19) | 0.81 (0.16) | | |

nn3)
NO/ONOO⁻ (1 μM LA-419) Caucasian

| | | | | |
|---|---|---|---|---|
| Genotype | Reference (7) | 1.63 (0.72) | 0.6937 | 0.49 |
| | Combination (19) | 1.61 (0.77) | | |

t statistic generated from the Non-parametric Mann-Whitney U test

Fold Difference Minor Allele Vs. Major Allele and Heterozygotes

TABLES oo1-oo3
Minor Allele vs. Major and Heterozygotes
Fold difference LA-419 5 μM
Fold difference = (LA-419 5 μM)/(control)

| | | | | |
|---|---|---|---|---|
| | | Fold difference | | |
| Variable | Level (n) | (mean ± sd) | t statistic | p value |

oo1)
NO (5 μM) Minor Allele vs. Major and Heterozygotes

| | | | | |
|---|---|---|---|---|
| G894T variant | T/T (5) | 1.12 (0.07) | −1.0734 | 0.288 |
| | G/G & G/T (44) | 1.15 (0.06) | | |
| T-786C variant | C/C (4) | 1.12 (0.06) | −1.0772 | 0.287 |
| | T/T & T/C (45) | 1.15 (0.06) | | |
| Intron 4 variant | a/a (3) | 1.14 (0.04) | −0.2048 | 0.839 |
| | b/b, b/a, c/c (42) | 1.14 (0.07) | | |

oo2)
ONOO⁻ (5 μM) Minor Allele vs. Major and Heterozygotes

| | | | | |
|---|---|---|---|---|
| G894T variant | T/T (5) | 0.81 (0.08) | 0.3798 | 0.706 |
| | G/G & G/T (44) | 0.80 (0.07) | | |
| T-786C variant | C/C (4) | 0.79 (0.08) | −0.0913 | 0.928 |
| | T/T & T/C (45) | 0.81 (0.07) | | |
| Intron 4 variant | a/a (3) | 0.83 (0.03) | 0.7508 | 0.457 |
| | b/b, b/a, c/c (42) | 0.81 (0.07) | | |

oo3)
NO/ONOO⁻ (5 μM) Minor Allele vs. Major and Heterozygotes

| | | | | |
|---|---|---|---|---|
| G894T variant | T/T (5) | 1.39 (0.13) | −0.8753 | 0.386 |
| | G/G & G/T (44) | 1.44 (0.16) | | |
| T-786C variant | C/C (4) | 1.42 (0.13) | −0.0913 | 0.928 |
| | T/T & T/C (45) | 1.44 (0.16) | | |
| Intron 4 variant | a/a (3) | 1.37 (0.04) | −0.3868 | 0.701 |
| | b/b, b/a, c/c (42) | 1.43 (0.17) | | |

Fold change = avg 5 μM/avg Cntl. t statistic generated from the Non-parametric Mann-Whitney U test
Fold difference = avg 5 μM/avg Cntl. t statistic generated from the Non-parametric Mann-Whitney U test

TABLES pp1-pp3
Minor Allele vs. Major and Heterozygotes
Fold difference LA-419 1 μM
Fold difference = (LA-419 1 μM)/(control)

| | | | | |
|---|---|---|---|---|
| | | Fold difference | | |
| Variable | Level (n) | (mean ± sd) | t statistic | p value |

pp1)
NO (1 μM) Minor Allele vs. Major and Heterozygotes

| | | | | |
|---|---|---|---|---|
| G894T variant | T/T (5) | 1.07 (0.19) | −0.6771 | 0.502 |
| | G/G & G/T (44) | 1.13 (0.25) | | |
| T-786C variant | C/C (4) | 1.02 (0.12) | −1.4058 | 0.166 |
| | T/T & T/C (45) | 1.14 (0.25) | | |
| Intron 4 variant | a/a (3) | 0.95 (0.25) | −0.9783 | 0.333 |
| | b/b, b/a, c/c (42) | 0.95 (0.25) | | |

pp2)
ONOO⁻ (1 μM) Minor Allele vs. Major and Heterozygotes

| | | | | |
|---|---|---|---|---|
| G894T variant | T/T (5) | 0.75 (0.21) | −1.4368 | 0.157 |
| | G/G & G/T (44) | 0.95 (0.27) | | |
| T-786C variant | C/C (4) | 1.11 (0.44) | 0.8581 | 0.395 |
| | T/T & T/C (45) | 0.91 (0.25) | | |
| Intron 4 variant | a/a (3) | 1.10 (0.34) | 1.0238 | 0.312 |
| | b/b, b/a, c/c (42) | 0.91 (0.27) | | |

pp3)
NO/ONOO⁻ (1 μM) Minor Allele vs. Major and Heterozygotes

| | | | | |
|---|---|---|---|---|
| G894T variant | T/T (5) | 1.62 (0.82) | 0.8092 | 0.422 |
| | G/G & G/T (44) | 1.34 (0.64) | | |
| T-786C variant | C/C (4) | 1.03 (0.36) | −1.2598 | 0.214 |
| | T/T & T/C (45) | 1.39 (0.67) | | |
| Intron 4 variant | a/a (3) | 0.96 (0.45) | −1.1148 | 0.271 |
| | b/b, b/a, c/c (42) | 1.42 (0.69) | | |

Fold difference = avg 1 μM/avg Cntl. t statistic generated from the Non-parametric Mann-Whitney U test

TABLES qq1-qq3
Fold difference LA-419 5 μM. Comparison between
minor allele homozygote and major allele and heterozygotes
(reference vs. combination).
Reference group = T/T + C/C
Combination = G/G + T/T, G/G + T/C, G/G/+C/C, G/T + T/T,
G/T + T/C, G/T + C/C, T/T + T/C
Fold difference = (LA-419 5 μM)/(control)

| | | | | |
|---|---|---|---|---|
| | | Fold difference | | |
| Variable | Level (n) | (mean ± sd) | t statistic | p value |

qq1)
NO (5 μM LA-419) Minor Allele vs. Major and Heterozygotes

| | | | | |
|---|---|---|---|---|
| Genotype | Reference (3) | 1.12 (0.07) | −0.8549 | 0.397 |
| | Combination (46) | 1.14 (0.06) | | |

qq2)
ONOO⁻ (5 μM LA-419) Minor Allele vs. Major and Heterozygotes

| | | | | |
|---|---|---|---|---|
| Genotype | Reference (3) | 0.77 (0.08) | −0.5630 | 0.576 |
| | Combination (46) | 0.81 (0.07) | | |

qq3)
NO/ONOO⁻ (5 μM LA-419) Minor Allele vs.
Major and Heterozygotes

| | | | | |
|---|---|---|---|---|
| Genotype | Reference (3) | 1.46 (0.14) | 0.3545 | 0.725 |
| | Combination (46) | 1.44 (0.16) | | |

t statistic generated from the Non-parametric Mann-Whitney U test

TABLES rr1-rr3
Fold difference LA-419 1 μM. Comparison between
minor allele homozygote and major allele and heterozygotes
(reference vs. combination).
Reference group = T/T + C/C
Combination = G/G + T/T, G/G + T/C, G/G/+C/C, G/T + T/T,
G/T + T/C, G/T + C/C, T/T + T/C
Fold difference = (LA-419 1 μM)/(control)

| | | | | |
|---|---|---|---|---|
| | | Fold difference | | |
| Variable | Level (n) | (mean ± sd) | t statistic | p value |

rr1)
NO (1 μM LA-419) Minor Allele vs. Major and Heterozygotes

| | | | | |
|---|---|---|---|---|
| Genotype | Reference (3) | 1.07 (0.06) | −0.7715 | 0.444 |
| | Combination (46) | 1.13 (0.25) | | |

rr2)
ONOO⁻ (1 μM LA-419) Minor Allele vs. Major and Heterozygotes

| | | | | |
|---|---|---|---|---|
| Genotype | Reference (3) | 0.89 (0.06) | 0.0209 | 0.984 |
| | Combination (46) | 0.93 (0.28) | | |

rr3)
NO/ONOO⁻ (1 μM LA-419) Minor Allele vs.
Major and Heterozygotes

| | | | | |
|---|---|---|---|---|
| Genotype | Reference (3) | 1.20 (0.04) | −0.5213 | 0.605 |
| | Combination (46) | 1.37 (0.68) | | |

t statistic generated from the Non-parametric Mann-Whitney U test

eNOS Protein Abundance

TABLE ss
eNOS % change, mean response for all genotypes and races
(5 μM LA-419)
% Change = (avg. 5 μM − avg. control)/(avg. control) * 100
ss)
eNOS protein abundance (5 μM LA-419)

| | | % Change | Chi-sq | |
|---|---|---|---|---|
| Variable | Level (n) | (mean ± sd) | statistic | p value |
| G894T variant | G/G (26) | −1.07 (8.07) | 0.0050 | 0.998 |
| | G/T (18) | −0.79 (7.54) | | |
| | T/T (5) | −1.54 (6.56) | | |
| T-786C variant | T/T (22) | −0.64 (5.88) | 3.0416 | 0.219 |
| | T/C (23) | −0.37 (9.0) | | |
| | C/C (4) | −6.82 (6.09) | | |
| Intron 4 variant | b/b (31) | −1.78 (6.88) | 1.5653 | 0.667 |
| | b/a (10) | 0.68 (9.65) | | |
| | a/a (3) | 1.11 (10.89) | | |
| | c/c (1) | −5.66 (0) | | |
| Race | Asian (1) | −17.56 (0) | 15.1070 | 0.01† |
| | Black (12) | −5.95 (6.82) | | |
| | Caucasian (26) | 2.50 (6.89) | | |
| | Caucasian/Black (2) | −4.89 (0.49) | | |
| | Hispanic (6) | −1.16 (4.99) | | |
| | Not Reported (2) | −4.62 (0.81) | | |

Chi-sq statistic generated from the Non-parametric ANOVA Kruskal-Wallis test

TABLES tt1-tt2
Effect of genotype on eNOS within group control and
5 μM LA-419; protein abundance, pg/ug total protein

| | | | | |
|---|---|---|---|---|
| | | pg/ug | Chi-sq | |
| Variable | Level (n) | (mean ± sd) | statistic | p value |

tt1)
eNOS protein abundance (control)

| | | | | |
|---|---|---|---|---|
| G894T variant | G/G (26) | 6.32 (1.45) | 1.4382 | 0.487 |
| | G/T (18) | 5.82 (1.33) | | |
| | T/T (5) | 5.87 (1.83) | | |
| T-786C variant | T/T (22) | 6.19 (1.43) | 2.9222 | 0.232 |
| | T/C (23) | 5.79 (1.38) | | |
| | C/C (4) | 7.22 (1.54) | | |
| Intron 4 variant | b/b (31) | 5.89 (1.29) | 3.2139 | 0.360 |
| | b/a (10) | 5.97 (1.67) | | |
| | a/a (3) | 7.22 (1.85) | | |
| | c/c (1) | 7.83 (0) | | |

tt2)
eNOS protein abundance (5 μM LA-419)

| | | | | |
|---|---|---|---|---|
| G894T variant | G/G (26) | 6.20 (1.31) | 1.7547 | 0.416 |
| | G/T (18) | 5.73 (1.17) | | |
| | T/T (5) | 5.69 (1.44) | | |
| T-786C variant | T/T (22) | 6.11 (1.26) | 2.7507 | 0.253 |
| | T/C (23) | 5.73 (1.29) | | |
| | CC (4) | 6.67 (1.16) | | |
| Intron 4 variant | b/b (31) | 5.75 (1.13) | 4.6691 | 0.198 |
| | b/a (10) | 5.95 (1.56) | | |
| | a/a (3) | 7.17 (1.19) | | |
| | c/c (1) | 7.39 (0) | | |

Chi-sq statistic generated from the Non-parametric ANOVA Kruskal-Wallis test

TABLES uu1-uu2
Effect of genotype on eNOS protein abundance (pg/ug total
protein) within group control and 5 μM LA-419 - comparison
between major allele homozygotes and minor allele carriers

| | | | | |
|---|---|---|---|---|
| | | pg/ug | | |
| Variable | Level (n) | (mean ± sd) | t statistic | p value |

uu1)
eNOS protein abundance (control) Major Allele vs.
Minor Allele Carrier

| | | | | |
|---|---|---|---|---|
| G894T variant | G/G (26) | 6.32 (1.45) | −1.1519 | 0.255 |
| | G/T & T/T (23) | 5.83 (1.41) | | |
| T-786C variant | T/T (22) | 6.19 (1.43) | 0.4523 | 0.653 |
| | T/C & C/C (27) | 6.01 (1.47) | | |
| Intron 4 variant | b/b (31) | 5.89 (1.29) | 0.8949 | 0.376 |
| | b/a, a/a, c/c (14) | 6.37 (1.71) | | |

uu2)
eNOS protein abundance (5 μM) Major Allele vs. Minor Allele Carrier

| | | | | |
|---|---|---|---|---|
| G894T variant | G/G (26) | 6.20 (1.31) | −1.2922 | 0.203 |
| | G/T & T/T (23) | 5.72 (1.20) | | |
| T-786C variant | T/T (22) | 6.11 (1.26) | 0.8945 | 0.376 |
| | T/C & C/C (27) | 5.87 (1.30) | | |
| Intron 4 variant | b/b (31) | 5.75 (1.13) | 1.0910 | 0.281 |
| | b/a, a/a, c/c (14) | 6.31 (1.50) | | |

t statistic generated from the Non-parametric Mann-Whitney U test

TABLES vv1-vv2
Effect of genotype on eNOS protein abundance (pg/ug total protein) within
group control and 5 μM LA-419 (Individual allelic combination).
Reference group = G/G + T/T

| | | | | |
|---|---|---|---|---|
| | | pg/ug | Chi-sq | |
| Variable | Level (n) | (mean ± sd) | statistic | p value |

vv1)
eNOS protein abundance (control)

| | | | | |
|---|---|---|---|---|
| G894T + T786C | G/G + T/T (18) | 6.26 (1.42) | 8.4968 | 0.204 |
| variant | G/G + T/C (8) | 6.44 (1.61) | | |
| | G/G/+C/C (0) | — | | |
| | G/T + T/T (4) | 5.85 (1.64) | | |
| | G/T + T/C (13) | 5.62 (1.15) | | |
| | G/T + C/C (1) | 8.24 (0) | | |
| | T/T + T/C (2) | 4.36 (0.32) | | |
| | T/T + C/C (3) | 6.88 (1.69) | | |

vv2)
eNOS protein abundance (5 μM LA-419)

| | | | | |
|---|---|---|---|---|
| G894T + T786C | G/G + T/T (18) | 6.17 (1.24) | 6.7528 | 0.344 |
| variant | G/G + T/C (8) | 6.27 (1.56) | | |
| | G/G/+C/C (0) | — | | |
| | G/T + T/T (4) | 5.82 (1.49) | | |
| | G/T + T/C (13) | 5.59 (1.08) | | |
| | G/T + C/C (1) | 7.25 (0) | | |
| | T/T + T/C (2) | 4.52 (0.30) | | |
| | T/T + C/C (3) | 6.47 (1.34) | | |

Chi-sq statistic generated from the Non-parametric ANOVA Kruskal-Wallis test

TABLES xx1-xx2
Effect of genotype on eNOS protein abundance (pg/ug total
protein) within group control and 5 μM LA-419. Comparison
between minor allele homozygote and major allele and
heterozygotes (reference vs. combination).
Reference group = T/T + C/C
Combination = G/G + T/T, G/G + T/C, G/G/+C/C, G/T + T/T,
G/T + T/C, G/T + C/C, T/T + T/C

| | | | | |
|---|---|---|---|---|
| | | pg/ug | | |
| Variable | Level (n) | (mean ± sd) | t statistic | p value |

xx1)
eNOS protein abundance (control)

| | | | | |
|---|---|---|---|---|
| Genotype | Reference (18) | 6.26 (1.42) | 0.5911 | 0.557 |
| | Combination (31) | 5.99 (1.46) | | |

xx2)
eNOS protein abundance (5 μM LA-419)

| | | | | |
|---|---|---|---|---|
| Genotype | Reference (18) | 6.17 (1.24) | 0.9436 | 0.350 |
| | Combination (31) | 5.86 (1.30) | | |

t statistic generated from the Non-parametric Mann-Whitney U test

TABLE yy
eNOS protein abundance (pg/ug total protein), net increase in
5 μM LA-419 over control - comparison between genotypes
yy)
eNOS protein abundance (5 μM LA-419)

| | | Net increase | | |
|---|---|---|---|---|
| | | pg/ug | Chi-sq | |
| Variable | Level (n) | (mean ± sd) | statistic | p value |
| G894T variant | G/G (26) | −0.12 (0.50) | 0.1444 | 0.930 |
| | G/T (18) | −0.08 (0.46) | | |
| | T/T (5) | −0.18 (0.45) | | |
| T786C variant | T/T (22) | −0.08 (0.36) | 3.7371 | 0.154 |
| | T/C (23) | −0.06 (0.54) | | |
| | C/C (4) | −0.55 (0.47) | | |
| Intron 4 variant | b/b (31) | −0.14 (0.45) | 1.8758 | 0.599 |
| | b/a (10) | −0.02 (0.56) | | |
| | a/a (3) | −0.05 (0.69) | | |
| | c/c (1) | −0.44 (0) | | |

Net increase = avg 5 μM − avg Cntl.
Chi-sq statistic generated from the Non-parametric ANOVA Kruskal-Wallis test

TABLE zz
eNOS protein abundance (pg/ug total protein), net increase in 5 μM
LA-419 over control - comparison between major allele homozygotes
and minor allele carriers
zz)
eNOS protein abundance (5 μM LA-419)

| | | Net increase | | |
|---|---|---|---|---|
| | | pg/ug | | |
| Variable | Level (n) | (mean ± sd) | t statistic | p value |
| G894T variant | G/G (26) | −0.12 (0.50) | 0.1503 | 0.881 |
| | G/T & T/T (23) | −0.10 (0.45) | | |
| T786C variant | T/T (22) | −0.08 (0.36) | −0.1106 | 0.912 |
| | T/C & C/C (27) | −0.13 (0.55) | | |
| Intron 4 variant | b/b (31) | −0.14 (0.45) | 0.4536 | 0.652 |
| | b/a, a/a, c/c (14) | −0.05 (0.55) | | |

Net increase = avg 5 μM − avg Cntl.
t statistic generated from the Non-parametric Mann-Whitney U test

TABLE A
eNOS protein abundance (pg/ug total protein), net increase in 5 μM
LA-419 over control (Individual allelic combination). Reference
group = G/G + T/T
A)
eNOS protein abundance (5 μM LA-419 increase over Control)

| | | Net increase | | |
|---|---|---|---|---|
| | | pg/ug | Chi-sq | |
| Variable | Level (n) | (mean ± sd) | statistic | p value |
| G894T + T786C | G/G + T/T (18) | −0.09 (0.35) | 5.1150 | 0.5292 |
| variant | G/G + T/C (8) | −0.17 (0.77) | | |
| | G/G/+C/C (0) | — | | |
| | G/T + T/T (4) | −0.03 (0.46) | | |
| | G/T + T/C (13) | −0.03 (0.42) | | |
| | G/T + C/C (1) | −0.99 (0) | | |
| | T/T + T/C (2) | 0.17 (0.02) | | |
| | T/T + C/C (3) | −0.41 (0.46) | | |

Chi-sq statistic generated from the Non-parametric ANOVA Kruskal-Wallis test

TABLE B
eNOS protein abundance (pg/ug total protein), net increase in 5 μM
LA-419 over control. Comparison between major allele homozygote and
minor allele carriers (reference vs. all carriers combination).
Reference group = G/G + T/T.
Combination = G/G + T/C, G/G/+C/C, G/T + T/T, G/T + T/C,
G/T + C/C, T/T + T/C, T/T + C/C
B)
eNOS protein abundance (5 μM LA-419 increase over Control)

| | | Net increase | | |
|---|---|---|---|---|
| | | pg/ug | | |
| Variable | Level (n) | (mean ± sd) | t statistic | p value |
| Genotype | Reference (18) | −0.09 (0.35) | −0.1970 | 0.845 |
| | Combination (31) | −0.12 (0.53) | | |

t statistic generated from the Non-parametric Mann-Whitney U test

TABLE C
Fold difference eNOS protein abundance, LA-419 5 μM - comparison
between genotypes
Fold difference = (LA-419 5 μM)/(control)
C)
eNOS protein abundance (LA-419 5 μM)

| | | Fold difference | Chi-sq | |
|---|---|---|---|---|
| Variable | Level (n) | (mean ± sd) | statistic | p value |
| G894T variant | G/G (26) | 1.00 (0.08) | 0.0050 | 0.998 |
| | G/T (18) | 0.99 (0.08) | | |
| | T/T (5) | 0.98 (0.07) | | |
| T-786C variant | T/T (22) | 0.99 (0.06) | 3.0416 | 0.218 |
| | T/C (23) | 1.00 (0.09) | | |
| | C/C (4) | 0.93 (0.06) | | |
| Intron 4 variant | b/b (31) | 0.98 (0.07) | 1.5653 | 0.667 |
| | b/a (10) | 1.01 (0.10) | | |
| | a/a (3) | 1.01 (0.11) | | |
| | c/c (1) | 0.94 (0) | | |

Fold difference = avg 5 μM/avg Cntl.
Chi-sq statistic generated from the Non-parametric ANOVA Kruskal-Wallis test

TABLE D
Fold difference eNOS protein abundance, LA-419 5 μM - comparison
between major allele homozygotes and minor allele carriers
Fold difference = (LA-419 5 μM)/(control)
D)
eNOS protein abundance (5 μm)

| | | Fold difference | | |
|---|---|---|---|---|
| Variable | Level (n) | (mean ± sd) | t statistic | p value |
| G894T variant | G/G (26) | 0.99 (0.08) | 0.0000 | 1.000 |
| | G/T & T/T (23) | 0.99 (0.07) | | |
| T-786C variant | T/T (22) | 0.99 (0.06) | 0.0000 | 1.000 |
| | T/C & C/C (27) | 0.99 (0.09) | | |
| Intron 4 variant | b/b (31) | 0.98 (0.07) | 0.6497 | 0.519 |
| | b/a, a/a, c/c (14) | 1.00 (0.09) | | |

Fold difference = avg 5 μM/avg Cntl.
t statistic generated from the Non-parametric Mann-Whitney U test

TABLE E
Fold difference eNOS protein abundance, LA-419 5 μM (Individual
allelic combination).
Reference group = G/G + T/T
Fold difference = (LA-419 5 μM)/(control)
E)
eNOS protein abundance (5 μM LA-419)

| | | Fold difference | Chi-sq | |
|---|---|---|---|---|
| Variable | Level (n) | (mean ± sd) | statistic | p value |
| G894T + T-786C | G/G + T/T (18) | 0.99 (0.06) | 4.6815 | 0.585 |
| variant | G/G + T/C (8) | 0.98 (0.12) | | |
| | G/G/+C/C (0) | — | | |
| | G/T + T/T (4) | 1.00 (0.08) | | |
| | G/T + T/C (13) | 1.00 (0.07) | | |
| | G/T + C/C (1) | 0.88 (0) | | |
| | T/T + T/C (2) | 1.04 (0.01) | | |
| | T/T + C/C (3) | 0.95 (0.06) | | |

Chi-sq statistic generated from the Non-parametric ANOVA Kruskal-Wallis test

TABLE F
Fold difference eNOS protein abundance, LA-419 5 μM. Comparison
between major allele homozygote and minor allele carriers (reference
vs. all carriers combination).
Reference group = G/G + T/T.
Combination = G/G + T/C, G/G/+C/C, G/T + T/T, G/T + T/C,
G/T + C/C, T/T + T/C, T/T + C/C
Fold difference = (LA-419 5 μM)/(control)
F)
eNOS protein abundance (5 μM LA-419)

| | | Fold difference | | |
|---|---|---|---|---|
| Variable | Level (n) | (mean ± sd) | t statistic | p value |
| Genotype | Reference (18) | 0.99 (0.06) | −0.0518 | 0.959 |
| | Combination (31) | 0.99 (0.09) | | |

t statistic generated from the Non-parametric Mann-Whitney U test

TABLE G
Fold difference eNOS protein abundance, LA-419 5 μM. Minor
Allele vs. Major Allele and Heterozygotes
Fold difference = (LA-419 5 μM)/(control)
G)
eNOS protein abundance (5 μM LA-419) Minor Allele Homozygotes vs.
Major Allele Homozygotes plus Heterozygotes

| | | Fold difference | | |
|---|---|---|---|---|
| Variable | Level (n) | (mean ± sd) | t statistic | p value |
| G894T variant | T/T (5) | 0.98 (0.07) | −0.0495 | 0.961 |
| | G/G & G/T (44) | 0.99 (0.08) | | |
| T786C variant | C/C (4) | 0.93 (0.06) | −1.6614 | 0.103 |
| | T/T & T/C (45) | 0.99 (0.08) | | |
| Intron 4 variant | a/a (3) | 1.01 (0.11) | 0.1138 | 0.910 |
| | b/b, b/a, c/c (42) | 0.99 (0.08) | | |

Fold difference = avg 5 μM/avg Cntl.
t statistic generated from the Non-parametric Mann-Whitney U test

TABLE H
Fold difference in eNOS protein abundance, LA-419 5 μM. Comparison
between minor allele homozygotes and major allele homozygotes plus
heterozygotes (reference vs. combination).
Reference group = T/T + C/C
Combination = G/G + T/T, G/G + T/C, G/G/+C/C, G/T + T/T,
G/T + T/C, G/T + C/C, T/T + T/C
Fold difference = (LA-419 5 μM)/(control)
H)
eNOS protein abundance (5 μM LA-419) Minor Allele Homozygotes vs.
Major Allele Homozygotes plus Heterozygotes

| | | Fold difference | | |
|---|---|---|---|---|
| Variable | Level (n) | (mean ± sd) | t statistic | p value |
| Genotype | Reference (3) | 0.95 (0.06) | −0.9800 | 0.332 |
| | Combination (46) | 0.99 (0.08) | | |

t statistic generated from the Non-parametric Mann-Whitney U test

TABLE I
Caucasian fold difference for eNOS protein abundance, LA-419 5 μM.
Comparison between genotypes:
Fold difference = (LA-419 5 μM)/(control)
I)
eNOS protein abundance (LA-419 5 μM) Caucasian

| | | Fold difference | Chi-sq | |
|---|---|---|---|---|
| Variable | Level (n) | (mean ± sd) | statistic | p value |
| G894T variant | G/G (10) | 1.06 (0.05) | 3.8950 | 0.14 |
| | G/T (13) | 1.00 (0.08) | | |
| | T/T (3) | 1.03 (0.02) | | |
| T-786C variant | T/T (10) | 1.03 (0.06) | 0.0564 | 0.97 |
| | T/C (15) | 1.02 (0.08) | | |
| | C/C (1) | 1.01 (0) | | |
| Intron 4 variant | b/b (18) | 1.00 (0.06) | 6.5351 | 0.038† |
| | b/a (5) | 1.07 (0.06) | | |
| | a/a (1) | 1.14 (0) | | |

Fold difference = avg 5 μM/avg Cntl.
Chi-sq statistic generated from the Non-parametric ANOVA Kruskal-Wallis test

TABLE J
Caucasian Fold difference in eNOS protein abundance, LA-419 5 μM.
Comparison between major allele homozygotes and minor allele carriers
Fold difference = (LA-419 5 μM)/(control)
J)
eNOS protein abundance (5 μM) Caucasian

| | | Fold difference | | |
|---|---|---|---|---|
| Variable | Level (n) | (mean ± sd) | t statistic | p value |
| G894T variant | G/G (10) | 1.06 (0.05) | 1.7656 | 0.09 |
| | G/T & T/T (16) | 1.01 (0.07) | | |
| T-786C variant | T/T (10) | 1.03 (0.06) | 0.1318 | 0.90 |
| | T/C & C/C (16) | 1.02 (0.08) | | |
| Intron 4 variant | b/b (18) | 1.00 (0.06) | 2.4333 | 0.02† |
| | b/a & a/a (6) | 1.08 (0.06) | | |

Fold change = avg 5 μM/avg Cntl. t statistic generated from the Non-parametric Mann-Whitney U test

TABLE K
Caucasian Fold difference in eNOS protein abundance LA-419 5 μM
(Individual allelic combination).
Reference group = G/G + T/T
Fold difference = (LA-419 5 μM)/(control)
K)
eNOS protein abundance (5 μM LA-419) Caucasian

| | | Fold difference | Chi-sq | |
|---|---|---|---|---|
| Variable | Level (n) | (mean ± sd) | statistic | p value |
| G894T + T-786C | G/G + T/T (7) | 1.04 (0.04) | 6.0565 | 0.30 |
| variant | G/G + T/C (3) | 1.10 (0.05) | | |
| | G/G/+C/C (0) | — | | |
| | G/T + T/T (3) | 1.02 (0.09) | | |
| | G/T + T/C (10) | 1.00 (0.08) | | |
| | G/T + C/C (0) | — | | |
| | T/T + T/C (2) | 1.04 (0.01) | | |
| | T/T + C/C (1) | 1.01 (0) | | |

Chi-sq statistic generated from the Non-parametric ANOVA Kruskal-Wallis test

TABLE L
Caucasian Fold difference in eNOS protein abundance, LA-419 5 μM.
Comparison between major allele homozygote and minor allele carriers
(reference vs. all carriers combination).
Reference group = G/G + T/T.
Combination = G/G + T/C, G/G/+C/C, G/T + T/T, G/T + T/C,
G/T + C/C, T/T + T/C, T/T + C/C
Fold difference = (LA-419 5 μM)/(control)
L)
eNOS protein abundance (5 μM LA-419) Caucasian

| | | Fold difference | | |
|---|---|---|---|---|
| Variable | Level (n) | (mean ± sd) | t statistic | p value |
| Genotype | Reference (7) | 1.04 (0.04) | 0.4625 | 0.65 |
| | Combination (19) | 1.02 (0.08) | | |

t statistic generated from the Non-parametric Mann-Whitney U test

TABLE M1-M4
% Change in Intron 4, 5 μM LA-419. Comparison between major allele
homozygotes and minor allele carriers

| | | | | |
|---|---|---|---|---|
| | | % Change | | |
| Variable | Level (n) | (mean ± sd) | t statistic | p value |

M1)
NO (5 μM LA-419)

| | | | | |
|---|---|---|---|---|
| Intron 4 variant | b/b (31) | 13.52 (6.80) | 1.2136 | 0.231 |
| | b/a, a/a, c/c (14) | 16.16 (6.09) | | |

M2)
ONOO⁻ (5 μM LA-419)

| | | | | |
|---|---|---|---|---|
| Intron 4 variant | b/b (31) | −19.19 (7.76) | −0.3555 | 0.724 |
| | b/a, a/a, c/c (14) | −19.27 (5.27) | | |

M3)
NO/ONOO⁻ (5 μM LA-419)

| | | | | |
|---|---|---|---|---|
| Intron 4 variant | b/b (31) | 42.24 (17.73) | 0.5516 | 0.584 |
| | b/a, a/a, c/c (14) | 44.37 (12.85) | | |

M4)
eNOS protein abundance (5 μM LA-419)

| | | | | |
|---|---|---|---|---|
| Intron 4 variant | b/b (31) | −1.78 (6.88) | 0.6497 | 0.519 |
| | b/a, a/a, c/c (14) | 0.32 (9.26) | | |

t statistic generated from the Non-parametric Mann-Whitney U test

TABLES N1-N3
% Change Intron 4, 1 μM LA-419. Comparison between major allele
homozygotes and minor allele carriers

| | | | | |
|---|---|---|---|---|
| | | % Change | | p |
| Variable | Level (n) | (mean ± sd) | t statistic | value |

N1)
NO (1 μM LA-419)

| | | | | |
|---|---|---|---|---|
| Intron 4 variant | b/b (31) | 13.46 (23.13) | 0.0000 | 1.00 |
| | b/a, a/a, c/c (14) | 11.76 (29.57) | | |

N2)
ONOO⁻ (1 μM LA-419)

| | | | | |
|---|---|---|---|---|
| Intron 4 variant | b/b (31) | −10.24 (27.17) | 0.7233 | 0.473 |
| | b/a, a/a, c/c (14) | −2.68 (29.78) | | |

N3)
NO/ONOO⁻ (1 μM LA-419)

| | | | | |
|---|---|---|---|---|
| Intron 4 variant | b/b (31) | 43.27 (71.09) | −0.4781 | 0.635 |
| | b/a, a/a, c/c (14) | 28.01 (62.22) | | |

t statistic generated from the Non-parametric Mann-Whitney U test

### REFERENCES

The following references, to the extent that they provide exemplary procedural or other details supplementary to those set forth herein, are specifically incorporated herein by reference.
U.S. Pat. No. 4,582,788
U.S. Pat. No. 4,627,429
U.S. Pat. No. 4,659,774
U.S. Pat. No. 4,683,194
U.S. Pat. No. 4,683,195
U.S. Pat. No. 4,683,202
U.S. Pat. No. 4,683,202
U.S. Pat. No. 4,683,202
U.S. Pat. No. 4,784,857
U.S. Pat. No. 4,800,159
U.S. Pat. No. 4,816,571
U.S. Pat. No. 4,883,750
U.S. Pat. No. 4,946,773
U.S. Pat. No. 4,959,463
U.S. Pat. No. 4,965,188
U.S. Pat. No. 5,126,145
U.S. Pat. No. 5,130,238
U.S. Pat. No. 5,141,813
U.S. Pat. No. 5,169,766
U.S. Pat. No. 5,264,566
U.S. Pat. No. 5,279,721
U.S. Pat. No. 5,428,148
U.S. Pat. No. 5,554,744
U.S. Pat. No. 5,574,146
U.S. Pat. No. 5,602,244
U.S. Pat. No. 5,605,798
U.S. Pat. No. 5,645,897
U.S. Pat. No. 5,662,925
U.S. Pat. No. 5,705,629
U.S. Pat. No. 5,788,983
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,483
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,770
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,337
U.S. Pat. No. 5,866,366
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,925,525
U.S. Pat. No. 5,928,862
U.S. Pat. No. 5,928,869
U.S. Pat. No. 5,928,870
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 5,952,174
U.S. Pat. No. 6,113,940
U.S. Pat. No. 4,656,127
U.S. Pat. No. 4,682,195
Ausubel et al., Current Protocols in Molecular Biology, John Wiley & Sons, NY, 1989.
Barany et al., Proc. Natl. Acad. Sci. USA, 88:189-193, 1991.
Bellus, J. Macromol. Sci. Pure Appl. Chem., A31(1): 1355-1376, 1994.
Burt et al., Hypertension, 25:305-313, 1995.
Campia et al., J. Am. Coll. Cardiol., 40:754-760, 2002.
de Arruda et al., Expert Rev. Mol. Diagn., 2(5):487-496, 2002.
Erbs et al., Eur. J. Cardiovasc. Prev. Rehabil., 13:826-831, 2006.
European Patent 201,184
European Patent 237,362
European Patent 258,017
European Patent 266,032
European Patent 320 308
European Patent 329,822
European Patent Appln. 50,424
European Patent Appln. 84,796
French Patent 2,650,840
Froehler et al., Nucleic Acids Res., 14:5399-5407, 1986.
Frohman, In: PCR Protocols: A Guide To Methods And Applications, Academic Press, N.Y., 1990.
Great Britain Appln. 2 202 328
Halushka et al., Nat. Genet., 22(3):239-247, 1999.
Harrison, J. Clin. Invest., 100:2153-2157, 1997.
Humphries et al., In: Molecular Diagnosis of Genetic Diseases, Elles (Ed.), 321-340, 1996.
Ignarro et al., Proc. Natl. Acad. Sci. USA, 84:9265-9269, 1987.
Imamura et al., Can. J. Cardiol., 20:1229-1234, 2004.
Inazuka et al., Genome Res, 7(11):1094-1103, 1997.
Innis et al., Proc. Natl. Acad. Sci. USA, 85(24):9436-9440, 1988.
Johnson et al., Nat. Genet., 29(2):233-237, 2001.
Jones, Nature, 199:280-282, 1963.
Kalinowski et al., Circulation, 109:2511-2517, 2004.
Ke and Cardon Bioinformatics, 19(2):287-288, 2003.
Klaassen's The Pharmacological Basis of Therapeutics
Komher, et al., Nucl. Acids. Res. 17:7779-7784, 1989.
Kuppuswamy et al., Proc. Natl. Acad. Sci. USA, 88:1143-1147, 1991.
Kwoh et al., Proc. Natl. Acad. Sci. USA, 86:1173, 1989.
Kwok and Chen, Curr Issues Mol. Biol., April; 5(2):43-60, 2003.
Kwok et al., Genomics, 23(1): 138-144, 1994.
Kwok et al., Genomics, 31(1):123-6, 1996.
Kwok, Annu. Rev. Genomics Hum. Genet., 2:235-258, 2001.
Landegren et al., Science 241:1077-1080, 1988.
Li et al., Am. J. Hypertens., 17:560-567, 2004.
Lu et al., Biopolymers, 73:606-613, 2004.
Lvovich and Scheeline, Anal. Chem., 69:454-462, 1997.
Malinski and Taha, Nature, 358:676-678, 1992.
Mason et al., Circulation, 112:3795-3801, 2005.
Maxam et al., Proc. Natl. Acad. Sci. USA, 74:560, 1977.
Megson et al., Curr. Opin. Investig. Drugs, 10(3):276-85, 2009.
Meyers, et al., Science, 230:1242, 1985.
Modrich, Ann. Rev. Genet., 25:229-253, 1991.
Mullis et al., Cold Spring Harbor Symp. Quant. Biol. 51:263-273, 1986.
Münzel et al., J. Clin. Invest., 95:187-194, 1995.
Napoli et al., Nitric Oxide, 15 265-279, 2006.
Nickerson et al., Proc. Natl. Acad. Sci. USA, 87:8923-8927, 1990.
Nyren et al., Anal. Biochem. 208:171-175, 1993.
Ohara et al., Proc. Natl. Acad. Sci. USA, 86:5673-5677, 1989.
Orita et al., Genomics, 5:874-879, 1989.
Paniagua et al., Circulation, 103:1752-1758, 2001.
Panza et al., N. Engl. J. Med., 323:22-27, 1990.
PCT Appln. PCT/US87/00880
PCT Appln. PCT/US89/01025
PCT Appln. WO 88/10315
PCT Appln. WO 89/06700
PCT Appln. WO 93/22456
PCT Appln. WO 95/11995
PCT Appln. WO89/06700
PCT Appln. WO90/01069
PCT Appln. WO91/02087
PCT Appln. WO92/15712
Physicians Desk Reference
Prezant et al., Hum. Mutat., 1:159-164, 1992.
Rees et al., Proc. Natl. Acad. Sci. USA, 86:3375-3378, 1989.
Remington's Pharmaceutical Sciences” 15th Edition, pages 1035-1038 and 1570-1580, 1990.
Ruano et al., Nucl. Acids Res., 19:6877-6882, 1991.
Ruano et al., Nucl. Acids Res., 17:8392, 1989.
Sambrook et al., In: Molecular cloning, Cold Spring Harbor Laboratory Press, Cold Spring Harbor, N.Y., 2001.
Sanger et al., J. Molec. Biol., 94:441, 1975.
Sheffield et al., Proc. Natl. Acad. Sci. USA, 86:232-236, 1989.
Small et al., N. Engl. J. Med., 347:11.35-1142, 2002.
Sokolov, Nucl. Acids Res. 18:3671, 1990.
Stevens et al., Biotechniques, 34:198-203, 2003.
Syvanen et al., Genomics 8:684-692, 1990.
Syvanen et al., Genomics 8:684-692, 1990.
Taddei et al., Hypertension, 21:929-933, 1993.
Taillon-Miller et al., Genome Res, 8(7):748-754, 1998.
The Merck Index, Eleventh Edition
Turki et al., J. Clin. Invest., 95:1635-1641, 1995.
Ugozzoll et al., GATA 9:107-112, 1992.
Walker et al., Proc. Natl. Acad. Sci. USA, 89:392-396, 1992.
Wartell et al., Nucl. Acids Res., 18:2699-2706, 1990.
Winter et al., Proc. Natl. Acad. Sci. USA, 82:7575, 1985.

1. A method for treating a cardiovascular disease or condition in a patient comprising administering to the patient an effective amount of pharmaceutical composition comprising the compound S-(6-Nitro-oxi-hexahydro-furo[3,2-b]furan-3-1-il)thioacetate after the patient is tested and determined to be homozygous wildtype (G/G) at position 894 in the endothelial nitric oxide synthase (NOS3) gene.

2. The method of claim 1, wherein the cardiovascular disease or condition is peripheral arterial disease, heart failure, coronary heart disease, coronary spasm, myocardial infarction, atherosclerosis, or pulmonary arterial hypertension.

3. The method of claim 1, wherein the patient is administered at least one dose of the composition, wherein the composition comprises about 1-40 mg of the compound.

4. The method of claim 3, wherein the patient is administered at least one dose of the composition, wherein the composition comprises about 5-20 mg of the compound.

5. The method of claim 3, wherein the patient is administered multiple doses of the composition.

6. The method of claim 5, wherein the patient is administered multiple doses of the composition in a 24 hour period.

7. The method of claim 5, wherein the patient is administered 1 to 3 doses of the composition, wherein the composition comprises about 1-40 mg of the compound.

8. The method of claim 1, further comprising providing or furnishing a biological sample from the patient for testing to determine the patient's genotype at position 894 in the NOS3 gene.

9. The method of claim 1, further comprising ordering a test that determines the patient's genotype at position 894 in the NOS3 gene.

10. A method for treating a patient with S-(6-Nitro-oxi-hexahydro-furo[3,2-b]furan-3-1-il)thioacetate comprising administering to the patient an effective amount of S-(6-Nitro-oxi-hexahydro-furo[3,2-b]furan-3-1-il)thioacetate after the patient is tested and determined to be homozygous wildtype (G/G) at position 894 in the endothelial nitric oxide synthase (NOS3) gene.

11. A method for increasing nitric oxide (NO) levels in a patient comprising administering to the patient an effective amount of S-(6-Nitro-oxi-hexahydro-furo[3,2-b]furan-3-1-il)thioacetate after the patient is tested and determined to be homozygous wildtype (G/G) at position 894 in the endothelial nitric oxide synthase (NOS3) gene.

12. The method of claim 11, wherein the patient has been diagnosed with a disease or condition associated with NO production.

13. The method of claim 12, wherein the disease is an ischemic cardiovascular disorder, glaucoma, or an intestinal disorder.

14.-19. (canceled)
